(19) United States
(12) Patent Application Publication
(10) Pub. No.: US 2004/0157240 A1
(43) Pub. Date: Aug. 12, 2004

(54) Diagnostic assay and related products

(75) Inventors: Scott T. Weiss, Chestnut Hill, MA (US); Kelan Tantisira, W. Roxbury, MA (US)

Correspondence Address: WOLF GREENFIELD & SACKS, PC, FEDERAL RESERVE PLAZA, 600 ATLANTIC AVENUE, BOSTON, MA, 02210-2211, US

(73) Assignee: The Brigham & Women's Hospital, Inc., Boston, MA (US)

(21) Appl. No.: 10/681,818

(22) Filed: Oct. 8, 2003

Related U.S. Application Data
(60) Provisional application No. 60/416,969, filed on Oct. 8, 2002.

(51) Int. Cl. C12Q 1/68

(52) U.S. Cl. 435/6

(57) ABSTRACT

This invention relates, in part, to methods of assessing sensitivity to a therapeutic agent in subjects with a disease. The invention also provides methods of detecting variant nucleic acid molecules and mutant proteins that correlate with treatment response. The invention in other aspects relates to kits and microarrays for assessing treatment sensitivity. The invention further provides novel variant nucleic acid molecules and the proteins they encode. The therapeutic agents include corticosteroids and beta-agonsists. Subjects suffering from a disease include those with COPD, asthma or depression.

### RELATED APPLICATIONS

This application claims the benefit under 35 U.S.C. § 119 of U.S. provisional application 60/416,969, filed Oct. 8, 2002, which is incorporated herein by reference.

### GOVERNMENT SUPPORT

Aspects of the invention may have been made using funding from National Institutes of Health Grant number NIH HL65899. Accordingly, the Government may have rights in the invention.

### FIELD OF THE INVENTION

This invention relates, in part, to methods of assessing sensitivity to a therapeutic agent in subjects. In particular, the invention provides methods of assessing the sensitivity of subjects to therapeutic agents, such as corticosteroids and beta-agonists (i.e., bronchodilators). The therapeutic agents can be used for treatment of respiratory disease and/or depression. For respiratory disease subjects, the invention provides methods of assessing sensitivity of subjects with chronic obstructive pulmonary disease or asthma to therapeutic agents, such as corticosteroids and beta-agonists. The invention also provides methods of detecting variant nucleic acid molecules and mutant proteins that correlate with treatment response. The invention in other aspects relates to kits and microarrays for assessing treatment sensitivity. The invention further provides novel variant nucleic acid molecules and the proteins they encode.

### BACKGROUND OF THE INVENTION

Chronic obstructive pulmonary disease (COPD) are a classification of respiratory diseases characterized by obstructed air flow. In 2000, approximately 10 million adults in the United States reported being diagnosed with COPD (Mannino, et al., Morbidity and Mortality Weekly Report, 51(SS06);1-16, 2002). In that same year, COPD resulted in millions of physician office and hospital visits, as well as 119,000 deaths (Mannino, et al., Morbidity and Mortality Weekly Report, 51(SS06);1-16, 2002). According to the International Classification of Diseases, Ninth Revision, COPD includes the following respiratory disorders: emphysema, bronchitis (not specified as acute or chronic), chronic bronchitis, bronchiectasis, extrinsic allergic alveolitis and chronic airways obstruction (not elsewhere classified). Additionally, COPD overlaps with asthma.

Asthma is the most common chronic disease of childhood in the developed world affecting about 12 million U.S. children under the age of sixteen (1). Ninety percent of all asthma, including asthma in adults, has its origins in childhood. Of prevailing concern are the recent increases in asthma self-reported prevalence (2) and hospitalization rates (3). Between 1980 and 1994, the self-reported prevalence of asthma increased from 30.7 to 53.8 per 1000, an increase of 75% (2). This increase has been accompanied by a similar increase in health care utilization and mortality over the same time period (2).

An estimated 12.6 billion dollars were spent in the U.S. in 1998, of which 58% were direct medical expenditures (DMEs) (4). Medication costs are currently the largest component of DMEs. Despite the availability of several classes of therapeutic agents for asthma, it has been estimated that as many as one-half of asthmatic patients do not respond to treatment with β2-agonists, leukotriene antagonists, or inhaled corticosteroids (5-7). As a whole, adverse drug reactions are estimated to cost the U.S. $100 billion and over 100,000 deaths a year (8).

### SUMMARY OF THE INVENTION

Sequence variations in pathway candidate genes and the relationship of these sequence variations to a subject's sensitivity to a therapeutic agent, have been discovered. Methods of assessing sensitivity to a therapeutic agent, such as corticosteroids or beta-agonists are provided. Methods of assessing sensitivity to a therapeutic agent in subjects with COPD or asthma are also provided. In one aspect of the invention, a method of determining a genotype of the subject is provided. The “genotype” of the subject is defined by a nucleotide sequence of a region of at least one gene selected from the following: ALOX15, CRH, CRHR1, CRHR2, urocortin, stresscopin, SRP (stresscopin-related peptide), CRHBP, EGR1, GATA3, HSD11B1, HSD11B2, MAPK8, NFATC4, NR3C1, SCYA11 (Eotaxin), FCER2 (CD23), IL18BP, ACTH (POMC), STAT3, STAT5A, STAT6, TBX21 (TBET) and TGF-β. In some embodiments the genotype of the subject is defined by a nucleotide sequence of a region of CRHR1. In other embodiments the genotype of the subject is defined by a nucleotide sequence of a region of FCER2. In still other embodiments the genotype of the subject is defined by a nucleotide sequence of a region of IL18BP. In yet other embodiments the genotype of the subject is defined by a nucleotide sequence of a region of CRHR2. The genotype of the subject can include the nucleotide sequence of one or more of the regions of one or more of the genes provided herein and can indicate the presence or absence of the one or more sequence variations which are indicative of sensitivity to the therapeutic agent. In some embodiments the genotype of the subject is defined by at least one nucleotide sequence selected from the group consisting of the nucleotide sequences set forth as SEQ ID NOs: 1-374 and fragments thereof, and combinations thereof.

The one or more sequence variations can be any change to a nucleotide sequence. Changes to a nucleotide sequence include base pair substitutions, insertions, deletions, and splice variants. In one embodiment, the sequence variation is a single nucleotide polymorphism. In other embodiments the sequence variation is a deletion. In still other embodiments the sequence variation is an insertion. One or more sequence variations may be determined by genotyping a subject. The subjects can be homozygous or heterozygous for these sequence variations. In some embodiments the one or more sequence variations are genetically linked and, therefore, indicate the haplotype of the subject. In some embodiments the haplotype is represented by various combinations of the sequences provided as SEQ ID NOs: 1-204 and 223-374. In other embodiments the haplotype is represented by SEQ ID NOs: 205-222. In yet other embodiments the haplotype is defined by the various combinations of the polymorphic sequences 1) rs1876828, rs242939, rs242941; 2) G9782a12, G9782a19, G9782a26, G9782a5, G9782a8 or 3) rs1892919, G9772a3, G9772a6.

Determining the genotype of the subject can be accomplished by any of a variety of standard methods that are well known in the art. In one embodiment, the presence or absence of a sequence variation that determines the subject's genotype is determined with nucleic acid hybridization. In some embodiments, the nucleic acid hybridization is accomplished with one or more probes. Probes, as provided herein, may be but are not limited to small molecules, proteins, peptides, nucleic acid molecules, and peptide nucleic acid (PNA) molecules.

Probes as provided herein may be bound to a solid substrate. In some aspects of the invention, nucleic acid probes are provided which hybridize under stringent conditions to a nucleic acid molecule provided herein. In some embodiments, the probe is bound to a detectable label. In still other embodiments the detectable label is a fluorescent, chemiluminescent or radioactive molecule. In yet other embodiments, the probe is bound to a solid substrate. Therefore, in other embodiments, hybridization is accomplished with nucleic acid microarrays.

In another embodiment of the invention, the presence or absence of the sequence variation is determined with nucleic acid amplification. Any of a variety of methods of amplification may be used and are well known in the art. Some of these method include but are not limited to direct RNA amplification, reverse transcription of RNA to cDNA, real-time (RT)-PCR, amplification of cDNA, anchor PCR, RACE PCR, and LCR (ligation chain reaction), etc. In other related embodiments, the amiplification is accomplished with polymerase chain reaction (PCR). In still other embodiments, the PCR is RT-PCR or real time PCR.

The methods of the invention provide the ability to assess a subject's sensitivity to a therapeutic agent. In one embodiment, the therapeutic agent is a corticosteroid. In another embodiment, the therapeutic agent is a beta-agonist (i.e. bronchodilator). In still other embodiments, the therapeutic agent may be an inhaled corticosteroid. Therapeutic agents may be any agent which acts on the corticosteroid or beta-agonist pathways. Further the therapeutic agents may be administered by standard methods known in the art in oral (oral solutions, syrups, tablets, effervescent tablets, extended release tablets etc.), inhaled (with a metered dose inhaler, nebulizer, dry powder inhaler, etc.) and injectable formulations.

In some embodiments, the sensitivity to these therapeutic agents can be assessed in subjects having or suspected of having any disease for which the administration of these therapeutic agents has some benefit. In some embodiments, the sensitivity to these therapeutic agents can be assessed in subjects having or suspected of having a COPD. In some embodiments, the COPD is chronic bronchitis, emphysema, bronchioectasis or extrinsic allergic alveolitis. In other embodiments, the sensitivity to these therapeutic agents can be assessed in subjects suffering or suspected of suffering from depression.

As used herein, “sensitivity” refers to the degree in which the intended response to a therapeutic agent is elicited when the agent is administered to the subject. In some embodiments, a subject's sensitivity to a therapeutic agent can be exhibited by a positive response to a therapeutic agent (i.e. a response that is the desired effect of the agent). In other embodiments, the reponse to a therapeutic agent is exhibited by a negative response (i.e. the agent results in a response that is not desired). In still other embodiments, a subject's sensitivity is exhibited by no response to a therapeutic agent. Therefore, in some embodiments, the genotype is indicative that the subject is not sensitive to the therapeutic agent.

Methods of assessing a subject's sensitivity to a therapeutic agent can further comprise assessing a risk factor in addition to the subject's genotype. In some embodiments, risk factors which indicate a subject's sensitivity include but are not limited to baseline level of lung function, gender, age, race and prior use of a particular therapeutic agent. In some embodiments the prior use of a particular therapeutic agent is the prior steroid use. Risk factors can be assessed prior to, concurrent with or subsequent to genotyping the subject. In these embodiments, the risk factors provide additional information to assess the subject's sensitivity and determine the appropriate therapeutic agent to treat the subject.

Another aspect of the invention is provided, whereby sensitivity to a therapeutic agent in a subject with a disease, such as COPD or asthma is assessed by determining a genotype of the subject, wherein the genotype of the subject is defined by a nucleotide sequence of a region of NR3C1, and wherein the presence or absence of a sequence variation in the region of NR3C1 is indicative of sensitivity to the therapeutic agent is provided. In one embodiment, the nucleotide sequence is selected from the group consisting of nucleotide sequences set forth as SEQ ID NOs: 89-121 and 309-324. In another embodiment, the nucleotide sequence is selected from the group consisting of nucleotide sequences set forth as SEQ ID NOs: 89 and 100. Other embodiments of this aspect of the invention are provided above.

In yet another aspect of the invention a method of assessing sensitivity to a therapeutic agent in a subject with a disease, such as COPD or asthma, is provided by detecting the presence of a nucleic acid molecule in a biological sample from the subject, wherein the nucleic acid molecule is selected from the following: (a) nucleotide sequences of a region of a gene selected from the group consisting of: ALOX15, CRH, CRHR1, CRHR2, urocortin, stresscopin, SRP (stresscopin-related peptide), CRHBP, EGR1, GATA3, HSD11B1, HSD11B2, MAPK8, NFATC4, SCYA11 (Eotaxin), FCER2 (CD23), IL18BP, ACTH (POMC), STAT3, STAT5A, STAT6, TBX21 (TBET) and TGF-β, which contains a sequence variation, (b) nucleotide sequences set forth as SEQ ID NOs: 1-88, 122-308 and 325-374 and (c) fragments of (a), wherein the fragment of the nucleotide sequences contains a sequence variation, and wherein the presence or absence of a sequence variation in the nucleic acid molecule is indicative of sensitivity to the therapeutic agent.

In some embodiments, the nucleic acid molecule is selected from the group consisting of nucleotide sequences set forth as SEQ ID NOs: 1-88, 122-308 and 325-374 and fragments thereof, which contain a sequence variation. In other embodiments, the nucleic acid molecule is selected from the group consisting of nucleotide sequences of a region of CRHR1, wherein the nucleotide sequence is selected from the group consisting of SEQ ID NOs: 205-208. In still other embodiments, the nucleic acid molecule is selected from the group consisting of nucleotide sequences of a region of IL18BP, wherein the nucleotide sequence is selected from the group consisting of SEQ ID NOs: 209-212. In still further embodiments, the nucleic acid molecule is selected from the group consisting of nucleotide sequences of a region of FCER2, wherein the nucleotide sequence is selected from the group consisting of SEQ ID NOs: 213-222. In other embodiments the nucleic acid molecule is selected from the group consisting of nucleotide sequences selected from the group consisting of nucleotide sequences set forth as SEQ ID NOs: 1-35.

In yet another embodiment, the nucleic acid molecule is selected from the group consisting of nucleotide sequences of a region of CRHR1, wherein the sequence comprises the polymorphisms of rs1876828, rs242939 and rs242941. Instill another embodiment, the nucleic acid molecule is selected from the group consisting of nucleotide sequences of a region of IL18BP, wherein the sequence comprises the polymorphisms of rs1892919, G9772a3 and G9772a6. In still other embodiments, the nucleic acid molecule is selected from the group consisting of nucleotide sequences of a region of FCER2, wherein the sequence comprises the polymorphisms of G9782a12, G9782a19, G9782a26, G9782a5 and G9782a8.

In some embodiments, the presence or absence of one or more sequence variations in a nucleic acid molecule is determined by first obtaining a biological sample. In some embodiments, the biological sample is a blood sample. In other embodiments, the biological sample may be cells, tissue or other body fluids, such as lymph node fluid, serum, etc. In still other embodiments, the nucleic acid molecule can be DNA or RNA. In still further embodiments, the nucleic acid molecules are genomic DNA, cDNA, or mRNA.

In another aspect of the invention, a nucleic acid microarray comprising at least two different nucleic acid molecules that hybridize to a nucleotide sequence selected from the following: (a) nucleotide sequences of a region of a gene selected from the group consisting of: ALOX15, CRH, CRHR1, CRHR2, urocortin, stresscopin, SRP (stresscopin-related peptide), CRHBP, EGR1, GATA3, HSD11B1, HSD11B2, MAPK8, NFATC4, SCYA11 (Eotaxin), FCER2 (CD23), IL18BP, ACTH (POMC), STAT3, STAT5A, STAT6, TBX21 (TBET) and TGF-β, which contain a sequence variation, (b) nucleotide sequences set forth as SEQ ID NOs: 1-88, 122-308 and 325-374 and (c) fragments of (a), wherein the fragment of the nucleotide sequence contains a sequence variation, and wherein the at least two nucleic acid molecules are fixed to a solid subtrate are provided. In one embodiment, the nucleotide sequence is selected from the group consisting of nucleotide sequences set forth as: SEQ ID NOs: 1-88, 122-308 and 325-374 and fragments thereof, which contain a sequence variation.

In another aspect, the gene is NR3C1. In one embodiment the nucleotide sequence is selected from nucleotide sequences set forth as SEQ ID NOs: 89-121 and 309-324. In yet another embodiment, the nucleotide sequence is selected from SEQ ID NO: 89 and 100.

In still other embodiments, the nucleic acid microarray includes at least ten different nucleic acid molecules fixed to a solid substrate. In yet another embodiment, the nucleic acid microarray includes at least one control nucleic acid molecule.

In another aspect of the invention, a method of assessing sensitivity to a therapeutic agent in a subject with a COPD or asthma is provided by determining the presence of a mutant protein encoded by a nucleic acid molecule selected from the following: (a) nucleotide sequences of a region of a gene selected from the group consisting of: ALOX15, CRH, CRHR1, CRHR2, urocortin, stresscopin, SRP (stresscopin-related peptide), CRHBP, EGR1, GATA3, HSD11B1, HSD11B2, MAPK8, NFATC4, SCYA11 (Eotaxin), FCER2 (CD23), IL18BP, ACTH (POMC), STAT3, STAT5A, STAT6, TBX21 (TBET) and TGF-β, which contains a sequence variation, (b) nucleotide sequences set forth as SEQ ID NOs: 1-88, 122-308 and 325-374, and (c) fragments of (b), which contain a sequence variation, wherein the presence of the mutant protein is indicative of sensitivity to the therapeutic agent. In some embodiments, the nucleic acid molecule is selected from the group consisting of nucleotide sequences set forth as SEQ ID NOs: 1-88, 122-308 and 325-374 and fragments thereof, which contain a sequence variation.

In another aspect, the gene is NR3C₁. In one embodiment the nucleotide sequence is selected from nucleotide sequences set forth as SEQ ID NOs: 89-121 and 309-324. In yet another embodiment, the nucleotide sequence is selected from SEQ ID NO: 89 and 100.

In other embodiments, the presence of the mutant protein is detected with an agent that selectively binds to the mutant protein. In some embodiments, the agent that selectively binds is a binding polypeptide. In still other embodiments, the binding polypeptide is an antibody or an antigen-binding fragment thereof. In other embodiments, the agent that selectively binds is a small molecule. These agents are, in some embodiments, bound to a detectable label. In some embodiments, the detectable label is a fluorescent molecule. In still other embodiments, the detectable label is a chemiluminescent or radioactive molecule. Other labeling molecules are well known in the art.

In addition to methods of assessing the sensitivity of a subject to a therapeutic agent, kits are provided. In one aspect of the invention a kit is provided that comprises one or more nucleic acid probes that hybridize to at least one nucleic acid molecule selected from the following: (a) nucleotide sequences of a region of a gene selected from the group consisting of: ALOX15, CRH, CRHR1, CRHR2, urocortin, stresscopin, SRP (stresscopin-related peptide), CRHBP, EGR1, GATA3, HSD11B1, HSD11B2, MAPK8, NFATC4, SCYA11 (Eotaxin), FCER2 (CD23), IL18BP, ACTH (POMC), STAT3, STAT5A, STAT6, TBX21 (TBET) and TGF-β, which contain a sequence variation, (b) nucleotide sequences set forth as SEQ ID NOs: 1-88, 122-308 and 325-374 and (c) fragments of (a), wherein the fragment of the nucleotide sequence contains a sequence variation. In this aspect of the invention, instructions for the use of the nucleic acid probes to correlate the presence of the at least one nucleic acid molecule with sensitivity to a therapeutic agent are included in the kit. In one embodiment, the nucleic acid molecule is selected from the group consisting of nucleotide sequences set forth as SEQ ID NOs: 1-88, 122-308 and 325-374.

In another aspect of the invention, a kit is provided which comprises one or more nucleic acid probes that hybridize to at least one nucleic acid molecule selected from the following: (a) nucleotide sequences of a region of a NR3C1, which contain a sequence variation, (b) nucleotide sequences set forth as SEQ ID NOs: 89-121 and 309-324 and (c) fragments of (a), wherein the fragment of the nucleotide sequence contains a sequence variation. In this aspect of the invention instructions, are also provided for the use of the nucleic acid probes to correlate the presence of the at least one nucleic acid molecule with sensitivity to a therapeutic agent. In one embodiment, the at least one nucleic acid molecule is selected from the group consisting of nucleotide sequences set forth as SEQ ID NOs: 89 and 100.

In another embodiment, the one or more nucleic acids consist of a first primer and a second primer, wherein the first primer and the second primer are constructed and arranged to selectively amplify a region of the nucleic acid molecule which contains a sequence variation. It is within the skill of the art to know the proper construction and arrangement of primers to selectively amplify a region of a nucleic acid molecules. Amplifications methods have been detailed herein.

In still another aspect of the invention, a kit is provided which comprises one or more binding polypeptides that selectively bind to a mutant protein encoded by a nucleic acid molecule selected from the following: (a) nucleotide sequences of a gene selected from the group consisting of: ALOX15, CRH, CRHR1, CRHR2, urocortin, stresscopin, SRP (stresscopin-related peptide), CRHBP, EGR1, GATA3, HSD11B1, HSD11B2, MAPK8, NFATC4, SCYA11 (Eotaxin), FCER2 (CD23), IL18BP, ACTH (POMC), STAT3, STAT5A, STAT6, TBX21 (TBET) and TGF-β, which contain a sequence variation, (b) nucleotide sequences set forth as SEQ ID NOs: 1-88, 122-308 and 325-374, and (c) fragments of (a) and (b), wherein the fragment of the nucleotide sequence contains a sequence variation, and instructions for the use of the one or more binding polypeptides to correlate the presence of the mutant protein with sensitivity to a therapeutic agent. In some embodiments, the nucleic acid molecule is selected from the group consisting of nucleotide sequences set forth as SEQ ID NOs: 1-88, 122-308 and 325-374 and fragments thereof, which contain a sequence variation. In one embodiment, the one or more binding polypeptides are antibodies or antigen-binding fragments thereof. In other aspects of the invention binding agents are provided which include small molecules in addition to binding polypeptides, such as antibodies or antigen-binding fragments thereof.

In another aspect, the mutant protein is encoded by NR3C1 which contains a sequence variation. In one embodiment the nucleotide sequence is selected from nucleotide sequences set forth as SEQ ID NOs: 89-121 and 309-324. In yet another embodiment, the nucleotide sequence is selected from SEQ ID NO: 89 and 100.

In other embodiments, the kits as provided herein optionally comprise one or more control agents. Additionally, in some embodiments the binding agents and, optionally, the one or more control agents are bound to a substrate.

The invention, therefore in some aspects also provides protein microarrays comprising one or more binding agents that bind to a mutant protein or fragment thereof encoded by the nucleic acids described herein. In some embodiments of the invention, one or more control peptide or protein molecules are attached to the substrate.

In other aspects of the invention, isolated nucleic acid molecules, the polypeptides they encode and compositions thereof are provided. In one aspect of the invention isolated nucleic acid molecules comprising (a) a nucleotide sequence set forth as SEQ ID NOs: 1-88, 122-308 and 325-374, (b) fragments of (a), wherein the fragment of the nucleotide sequence contains a sequence variation, and (c) complements of (a) and (b) are provided. In some embodiments the isolated nucleic acid molecules comprises a nucleotide sequence set forth as SEQ ID NOs: 1-88, 122-308 and 325-374.

In another aspect of the invention, the isolated nucleic acid molecules comprise (a) a nucleotide sequence set forth as SEQ ID NOs: 89-121 and 309-324, (b) fragments of (a), wherein the fragment of the nucleotide sequence contains a sequence variation, and (c) complements of (a) and (b) are provided.

In still another aspect of the invention isolated nucleic acid molecules, which comprise any of the nucleic acid molecules described herein as well as fragments and complements thereof. In some instances these isolated nucleic acid molecules comprise a nucleotide sequences set forth as SEQ ID NOs: 53-67, 76, 77, 89, 100, 131-135, 165-175, 253-262, 268, 287, 288, 294, 298-304, 309, 331-333 and 341-347. In other aspects of the invention isolated nucleic acid molecules that are the degenerate equivalents of any of the nucleic acid molecules described herein are also provided.

In some embodiments of the invention, expression vectors, host cells and the polypeptides produced from the expression vectors and/or host cells are provided.

These and other aspects of the invention will be described in further detail in connection with the detailed description of the invention.

### BRIEF DESCRIPTION OF THE DRAWINGS

FIG. 1 illustrates the effect of the FCER2 haplotype on the risk of hospitalization and emergency visits.

FIG. 2 illustrates the general methodologic approach. After identifying candidate genes of interest, variants were identified via DNA sequencing and public databases. SNPs were selected with preference for known functional variants, allele frequencies over 10%, and no fewer than every 10 kb apart. Genotyping was performed initially on our Adult Study and haplotype tagged SNPs were identified. Any gene with single allelic or haplotypic effects with significant (p<0.05) effects were then genotyped in our pediatric population (CAMP). Replicated findings were re-tested in our second adult population (ACRN) before our final, multivariate analysis.

FIG. 3 shows the heterogeneity of response to inhaled corticosteroids at 8 weeks (Adult Study and CAMP), and 6 weeks (ACRN). The distribution of responses within each population is approximately normal and suggests that other factors, including genetic, may be contributing to the therapeutic response.

FIG. 4 demonstrates the association of CRHR1 SNPs with longitudinal response to inhaled corticosteroids in asthmatics, adjusted for age, sex, height, and baseline FEV1. FIG. 4A illustrates that rs242941 is associated with the response over 8 weeks in two populations (Adult Study and CAMP). Individuals with the variant TT genotype demonstrated a significant improvement in lung function with corticosteroid use compared to those with the wild type CC genotype. FIG. 4B illustrates that rs1876828 is associated with the response over 6 weeks in the ACRN population. Individuals with the variant AA genotype demonstrated a significant improvement in lung function with corticosteroid use compared to those with the wild type GG genotype. Mean values±SEM are shown.

FIG. 5 illustrates the eight week response to inhaled corticosteroids, stratified by CRHR1 GAT haplotype status in the Adult Study and CAMP. Utilizing the htSNPs rs1876828, rs242939, and rs242941, the mean FEV1 improvement in those adults imputed with the GAT/GAT homozygous haplotype was 13.7%, while it was 5.5% in those homozygous for two non-GAT haplotypes. In CAMP, those imputed for the GAT/GAT haplotype demonstrated a 21.8% improvement in FEV1 vs. 7.4% for those with no GAT haplotype. Improvement in those heterozygous for the GAT haplotype was intermediate between the two groups, suggesting an additive effect. Mean values±SEM are shown.

### DETAILED DESCRIPTION

As there is large interindividual variation in the treatment response to various medications, treatment of subjects will improve with an understanding of the genetic basis of these diseases. For instance, such understanding can be useful for subjects with respiratory diseases, such as COPD and in particular, asthma. Asthma is a genetic disease, which affects over 155 million individuals in the developed world (38) and has been noted to cluster in families for over 3 centuries (12). Based on twin studies, the broad sense heritability estimates of asthma have ranged from 36% (13) to 75% (14). Common diseases such as asthma are complex diseases in that no single gene is causal by itself. Instead, such diseases likely result from the influence of multiple genetic, environmental, and developmental factors.

The variability of the treatment response in asthma is likewise complex (7), with a substantial proportion likely due to genetic factors (5). The study of the role of genetic determinants in the variable response to therapy is the basis of the field of pharmacogenetics. Ideally, pharmacogenetics will allow for “individualized therapy” based upon an individual's genetic make-up that will maximize the potential for therapeutic benefit, while minimizing the risk of adverse effects. The potential for cost savings and for decreasing morbidity and mortality is immense.

Pharmacogenetic studies in the field of asthma have focused on the β2-agonist and leukotriene antagonist pathways. In the β₂-agonist pathway, subjects homozygous Arg/Arg at position 16 of the β₂-adrenergic receptor gene significantly decrease their peak expiratory flow rates with regular utilization of albuterol therapy, compared to both those Arg/Arg receiving prn albuterol and those Gly/Gly at position 16 in either treatment group (17, 18). This may be a result of basal downregulation of the Gly/Gly subjects (19), since Arg/Arg children are more likely than Gly/Gly children to manifest an acute bronchodilator response to albuterol (20). In the leukotriene pathway, a microsatellite polymorphism of the ALOX5 gene has been identified. Lack of the wild-type allele has been associated with decrements in FEV₁ upon receipt of leukotriene antagonists in two different studies (21-23).

Although the symptoms of most asthmatics can be adequately controlled with therapy, there is large interindividual variation in the treatment response to each of the major classes of asthma medications (16, 36). The National Heart, Lung, and Blood Institute recommends the use of anti-inflammatory agents, including inhaled corticosteroids, for any asthmatic with persistent symptoms. While the cost-effectiveness of these agents is unquestioned in those asthmatics in whom they work (24), the potential for side effects in those on higher doses can be significant. These side effects can include growth retardation in children, adrenal suppression, bone demineralization, skin changes, and cataract formation. The heterogeneous response to inhaled steroid therapy includes a significant number of non-responders (16).

Of the agents available, inhaled corticosteroids are the most effective and commonly used drugs for the treatment of asthma but may be associated with serious adverse effects (39-41). Since the response to inhaled corticosteroid treatment in patients with asthma is highly repeatable (5), it may be that a genetic basis is the reason for the heterogeneity of therapeutic response. To date, however, there has been only modest success in identifying genes influencing treatment response in asthma (22) or other diseases (7).

To investigate the genetic contribution to the variation in response to inhaled corticosteroids, single nucleotide polymorphisms (SNPs) from biologic candidate genes in a clinical trial of adults with asthma were genotyped. SNPs were found to be associated with the eight-week change in lung function, measured by forced expiratory volume in one second (FEV₁). Follow-up testing of 308 asthmatic children revealed one SNP and one specific haplotype generated from three haplotype-tag SNPs (htSNPs) (37) in the corticotropin releasing hormone receptor 1 gene (CRHR1) associated with an enhanced response to inhaled corticosteroids in both the adult and childhood asthmatics over the same time frame. The homozygous haplotype was associated with over twice the improvement in FEV₁ in both populations compared to absence of this haplotype. The three CRHR1 htSNPs were subsequently tested in a third population of 339 adult asthmatics; one of the htSNPs was also strongly associated with corticosteroid response.

In one aspect, the invention relates, in part, to a method of assessing sensitivity to a therapeutic agent in a subject for which the therapeutic agent gives some benefit. In some aspects the subject suffers from an inflammatory disease. In another aspect the sensitivity is assessed in a subject with COPD. In another aspect of the invention, sensitivity is assessed in a subject with asthma. In still another aspect of the invention the sensitivity is assessed in a subject suffering from depression. Within the corticosteroid pathway of COPD or asthma therapy, genetic differences between individuals, as described herein, have been discovered that predict the relative odds of having a poor or enhanced response to therapy. As used herein, “sensitivity” refers to the degree in which the intended response to a therapeutic agent is elicited when the agent is administered to the subject. For example, a subject sensitive to an anti-inflammatory agent is said to exhibit a decrease in inflammation when administered the therapeutic agent. This decrease in inflammation may be a large decrease, moderate decrease or minimal decrease. In another example, the subject to which the anti-inflammatory agent is administered may exhibit no change due to the agent or may exhibit an increase in inflammation (i.e. a negative response, a response that is opposite of the intended effect). Such a subject is referred to as a subject that is not sensitive to the therapeutic agent

When sensitivity to a therapeutic is assessed according to the invention, it is assessed to determine whether a subject is sensitive to a therapeutic or is not sensitive to a therapeutic. Thus, when a particular parameter is determined to be indicative of sensitivity to a therapeutic agent, that particular parameter may be indicative of a subject that is sensitive to the therapeutic or a subject that is not sensitive to the therapeutic agent, depending on the parameter being assessed. For instance, the presence of a particular sequence variation in a gene of a subject may be indicative that the subject is sensitive to a therapeutic agent or is insensitive to a therapeutic agent, depending on the particular sequence variant and the gene. In other words, the subject can have a poor, an enhanced response or no response.

As used herein, a “subject” is a vertebrate and preferably a human, non-human primate, cow, horse, pig, sheep, goat, dog, cat or rodent. In some embodiments the subject has an inflammatory disease. In some embodiments, the subject has a COPD. In other embodiments the subject has asthma. A subject having COPD is a subject that has been diagnosed with a COPD or otherwise believed to have COPD or a subject at risk of developing or having COPD. As used herein, “chronic obstructive pulmonary disease” or “COPD” is a respiratory disorder characterized by chronic irreversible airflow obstruction and includes, for instance, emphysema, bronchitis (not specified as acute or chronic), chronic bronchitis, bronchiectasis, extrinsic allergic alveolitis, and chronic airways obstruction (not elsewhere classified). In some instances COPD and asthma overlap. In asthma, the air flow is obstructed due to a chronic inflammatory state of the airways. In some embodiments, the subjects of the invention are at risk of having or have asthma. In still other embodiments the subject is in need of corticosteroid therapy. In yet other embodiments the subject is at risk of or has been diagnosed or is otherwise believed to be suffering from depression.

The sensitivity of the subject is assessed using the methods described herein for a variety of therapeutic agents. As used herein, a therapeutic agent is any agent that has some utility in treating or preventing COPD or asthma. The therapeutic agent may be one which affects the corticosteroid pathway or β₂-agonist pathway, such as an inhaled corticosteroid. Examples of coricosteroids include but are not limited to Betamethasone, Budesonide, Cortisone, Dexamethasone, Flunisolide, Hydrocortisone, Methylprednisolone, Prednisolone, Prednisone, and Triamcinolone. Likewise, examples of beta-agonists (i.e. bronchodilators) include but are not limited to albuterol (Proventil, Ventolin), epinephrine (Primatene), ipratropium (Atrovent), metaproterenol (Alupent, Metaprel), and terbutaline (Brethine). Therapeutic agents may be administered by standard methods known in the art in oral (oral solutions, syrups, tablets, effervescent tablets, extended release tablets etc.), inhaled (with a metered dose inhaler, nebulizer, dry powder inhaler, etc.) and injectable formulations. Additional examples of corticosteroids and bronchodilators are provided in the Examples. Such a therapeutic agent is referred to as a corticosteroid or β₂-agonist therapeutic agent. Other therapeutic agents include but are not limited to bronchodilators.

In one aspect of the invention, sensitivity of a subject to a therapeutic agent as described above is assessed by determining the genotype of the subject. As used herein, the genotype of the subject is defined by a nucleotide sequence of at least one region of at least one gene. For the purposes of genotyping the subject in the methods described herein, the at least one region of the at least one gene is suspected to contain a sequence variation which is indicative of the sensitivity to the therapeutic agent. In some embodiments, the genotype of the subject is defined by sequence variations in more than one gene. In other embodiments the genotype is defined by at least one sequence variation in 2, 3, 4, 5, 6, 7, 8, 9, 10 or more genes. In still other embodiments the genotype is defined by more than one sequence variation in a single gene.

The term “sequence variation” refers to at least one nucleotide in a nucleic acid which is different than a nucleotide in a reference nucleic acid. A reference nucleic acid is any nucleic acid having a known nucleotide sequence. Sequence variations include but are not limited to base pair substitutions, insertions, deletions, and splice variants.

Sequence variations, therefore, include single nucleotide polymorphisms (SNPs). A SNP as used herein is a single base pair within a DNA region which exhibits variability from individual to individual. At the variable position in the SNP two alternative bases occur at a relatively high frequency (greater than 1%) in the human population. A “polymorphic region” is a region or segment of DNA which varies from individual to individual. The two DNA strands which are complementary to one another except at the variable position are referred to as alleles. A polymorphism is allelic because some members of a species carry one allele and other members carry a variant allele. When only one variant sequence exists, a polymorphism is referred to as a diallelic polymorphism. There are three possible genotypes in a diallelic polymorphic DNA. These three genotypes arise because it is possible that the DNA may be homozygous for one allele, homozygous for the other allele or heterozygous.

As used herein, a nucleic acid molecule which contains one or more sequence variations is a “sequence variant”. Sequence variants are provided herein which include single allelic variants and sequences with more than one sequence variation that are genetically linked (e.g. haplotype). The term “haplotype” includes more than one sequence variation within a single gene or within a set of linked genes. Thus the term “haplotype” as used herein, refers to an ordered combination of alleles in a defined genetic region that co-segregate. Such alleles are said to be “linked.” The alleles of the haplotype may be within a gene, between genes, or in adjacent genes or chromosomal regions that co-segregate with high fidelity. The term “linkage” refers to the degree to which regions of a nucleic acid are inherited together. DNA on different chromosomes are inherited together 50% of the time and do not exhibit linkage. The term “linkage disequilibrium” refers to the co-segregation of two alleles at a linked loci such that the frequency of the co-segregation of the alleles is greater than would be expected from separate frequencies of occurrence of each allele. In this aspect of the invention, the genotype of the subject is determined for at least one region of at least one gene selected from the group consisting of ALOX15, CRH, CRHR1, CRHR2, urocortin, stresscopin, SRP (stresscopin-related peptide), CRHBP, EGR1, GATA3, HSD11B1, HSD11B2, MAPK8, NFATC4, NR3C1, SCYA11 (Eotaxin), FCER2 (CD23), IL18BP, ACTH (POMC), STAT3, STAT5A, STAT6, TBX21 (TBET) and TGF-β. In another aspect of the invention the genotype is determined for a region of NR3C1.

The genotype of a subject for a single gene or set of genes can be determined with any of a number of methods that are well known to those of skill in the art. The genotype of the subject can be determined, for instance, using any standard sequencing or sequence analysis techniques. Examples of such techniques are described in Cotton, R. G. H., Mutation Detection, Oxford University Press, 1998. Some sequencing or sequence analysis techniques include direct sequencing, minisequencing, pyrosequencing (Ronaghi, et al., Science,281, 1998), PCR primer mismatch, single-base extension, restriction fragment length polymorphism, single stranded conformational analysis, and ligation assays in addition to a variety of other amplification and hybridization techniques.

The presence or absence of the sequence variation in a nucleic acid molecule may be determined, for instance, with amplification methods which include, but are not limited to: direct RNA amplification, reverse transcription of RNA to cDNA, real-time (RT)-PCR, amplification of cDNA, anchor PCR, RACE PCR, and LCR (ligation chain reaction), etc. The amplification may be a preliminary step performed in order to increase the number of nucleic acid molecules to be further analyzed. For instance, amplification can be combined with subsequent separation or detection procedures such as gel electrophoresis, capillary gel electrophoresis, mass spectrometry, and HPLC, etc.

For example, the genotyping can be performed using a SEQUENOM MassARRAY Matrix Assisted Laser Desorption and Ionization Time of Flight (MALDI-TOF) mass spectrometer (Sequenom, San Diego, Calif.). SEQUENOM MALDI-TOF mass spectrometer allows the analysis of unlabeled single-base extension minisequencing reactions. Primers for use in these minisequencing reactions can be designed with a variety of methods known in the art, including the semi-automated primer design program (Spectro DESIGNER, Sequenom). In this embodiment, very short extension method (VSET) (33) can be used to extend sequencing products by only one base for 3 or 4 nucleotides (due to the presence of dideoxynucleotides for 3 of the 4 nucleotides in the minisequencing reaction) and by several additional bases for the fourth nucleotide which are specified in advance to represent one of the two alleles at a given SNP locus. The allelic variants are then distinguished by mass separation with MALDI-TOF.

The detection of sequence variants can also be performed with any of a number of specific hybridization procedures well known in the art. A Southern blot may be performed using the foregoing conditions, together with a detectably labeled probe (e.g. radioactive, chemiluminescent or fluorescent probes). After washing the membrane to which the DNA is finally transferred, the membrane can be placed against X-ray film or analyzed using a phosphorimager device to detect the radioactive, fluorescent or chemiluminescent signal. Northern blot hybridizations using the foregoing conditions can also be performed on samples taken from subjects suspected of having or diagnosed as having a disease, such as COPD or asthma. Other hybridization techniques include FISH (fluorescent in situ hybridization), dot blot, slot bot analyses and microarrays.

Another hybridization technique is nucleic acid microarrays. Nucleic acid microarray technology, which is also known by other names including: DNA chip technology, gene chip technology, and solid-phase nucleic acid array technology, is well known to those of ordinary skill in the art and is based on, but not limited to, obtaining an array of identified nucleic acid probes on a fixed substrate, labeling target molecules with reporter molecules (e.g., radioactive, chemiluminescent, or fluorescent tags such as fluorescein, Cye3-dUTP, or Cye5-dUTP), hybridizing target nucleic acids to the probes, and evaluating target-probe hybridization. A probe with a nucleic acid sequence that perfectly matches the target sequence will, in general, result in detection of a stronger reporter-molecule signal than will probes with less perfect matches. Many components and techniques utilized in nucleic acid microarray technology are presented in The Chipping Forecast, Nature Genetics, Vol.21, January 1999, the entire contents of which is incorporated by reference herein.

Detection and identification of hybridized probes can also be determined with analytical separation techniques such as those listed above. In some embodiments, MALDI-TOF mass spectrometry is used. The use of MALDI-TOF to separate peptide nucleic acid (PNA) probe hybridization products has been described (Ross, et al., Anal. Chem., 69 (20):4197-202, 1997). In other embodiments, capillary electrophoresis is used for probe hybridization product separation (Basile, et al., Electrophoresis, 23 (6), 2002).

As used herein, probes which specifically hybridize to a sequence variant do so under sufficient hybridizing conditions. It is within the knowledge of one of skill in the art to be able to determine the hybridizing conditions necessary to detect one or more sequence variants in a sample. In a preferred embodiment, the probe hybridizes to the sequence variant under stringent conditions. In other embodiments, the probe hybridizes under highly stringent conditions. The term “stringent conditions” as used herein refers to parameters with which the art is familiar. Such parameters include salt, temperature, length of the probe, etc. The amount of resulting base mismatch upon hybridization can range from near 0% (“high stringency”) to about 30% (“low stringency”). Nucleic acid hybridization parameters may be found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. One example of high-stringency conditions is hybridization at 65° C. in hybridization buffer (3.5×SSC, 0.02% Ficoll, 0.02% polyvinyl pyrrolidone, 0.02% Bovine Serum Albumin, 2.5 mM NaH2PO4 (pH7), 0.5% SDS, 2 mM EDTA). SSC is 0.15M sodium chloride/0.15M sodium citrate, pH7; SDS is sodium dodecyl sulphate; and EDTA is ethylenediaminetetracetic acid. After hybridization, a membrane upon which the nucleic acid is transferred is washed, for example, in 2×SSC at room temperature and then at 0.1-0.5×SSC/0.1×SDS at temperatures up to 68° C.

A “probe” as used herein is any compound which specifically interacts with and identifies a sequence variation. For instance, a probe may be a nucleic acid, such as a complementary nucleic acid molecule, a protein or a peptide nucleic acid (PNA) molecule. The probes may be specific for a nucleotide sequence that contains a single sequence variation, or may specifically hybridize to a nucleotide sequence that contains 2, 3, 4, 5 or more sequence variations. One or more probes may be used to identify multiple sequence variations. For instance, one or more probes may specifically hybridize to a region of a nucleic acid molecule which indicates a haplotype. A set of probes may be used which are capable of hybridizing to more than one sequence variant in one or more genes. The probes may be of any length to specifically detect the sequence or sequences of interest. Nucleic acid probes are selected from the group of nucleic acids including, but not limited to: DNA, genomic DNA, cDNA, and oligonucleotides; and may be natural or synthetic. Oligonucleotide probes preferably are 20 to 25-mer oligonucleotides and DNA/cDNA probes preferably are 500 to 5000 bases in length, although other lengths may be used. In preferred embodiments, the probes are about 8, 9, 10, 11, 12, 13, 14, 15, 20, 25, 30, or more nucleotides in length. Appropriate probe length may be determined by one of ordinary skill in the art by following art-known procedures. Probes may be purified to remove contaminants using standard methods known to those of ordinary skill in the art such as gel filtration or precipitation. The probe or set of probes may optionally be attached to a solid substrate.

Therefore in one aspect of the invention a nucleic acid microarray is provided. In one embodiment, the microarray substrate may be coated with a compound to enhance synthesis of the probe or set of probes on the substrate. Such compounds include, but are not limited to, oligoethylene glycols. In another embodiment, coupling agents or groups on the substrate can be used to covalently link the first nucleotide or oligonucleotide to the substrate. These agents or groups may include, for example, amino, hydroxy, bromo, and carboxy groups. These reactive groups are preferably attached to the substrate through a hydrocarbyl radical such as an alkylene or phenylene divalent radical, one valence position occupied by the chain bonding and the remaining attached to the reactive groups. These hydrocarbyl groups may contain up to about ten carbon atoms, preferably up to about six carbon atoms. Alkylene radicals are usually preferred containing two to four carbon atoms in the principal chain. These and additional details of the process are disclosed, for example, in U.S. Pat. No. 4,458,066, which is incorporated by reference in its entirety. The nucleic acid probes may be synthesized directly on the substrate in a predetermined grid pattern using methods such as light-directed chemical synthesis, photochemical deprotection, or delivery of nucleotide precursors to the substrate and subsequent probe production.

Targets for microarrays include proteins or nucleic acids including but not limited to: DNA, genomic DNA, cDNA, RNA, mRNA and may be natural or synthetic.

In some embodiments of the invention, one or more control nucleic acid molecules are attached to the substrate. Preferably, control nucleic acid molecules allow determination of factors such as nucleic acid quality and binding characteristics, reagent quality and effectiveness, hybridization success, and analysis thresholds and success. Control nucleic acids may include but are not limited to expression products of genes such as housekeeping genes or fragments thereof.

In some aspects of the invention, the methods provided to assess the sensitivity to a therapeutic agent is determined by the presence or absence of one or more of the sequence variants described above in a sample obtained from a subject. As used herein, a “biological sample” includes, but is not limited to: tissue, cells, or body fluid (e.g. serum, blood, lymph node fluid, etc.). The fluid sample may include cells and/or fluid. The tissue and cells may be obtained from a subject or may be grown in culture (e.g. from a cell line). As used herein, a biological sample is body fluid, tissue or cells obtained from a subject using methods well-known to those of ordinary skill in the related medical arts. The tissue may be obtained from a subject or may be grown in culture (e.g. from a cell line).

In addition to the methods, kits are also provided that are useful for determining a subject's sensitivity to a therapeutic agent. One example of a kit of the invention is a kit that provides components necessary to determine the presence or absence of one or more sequence variants of the invention. Such components include probes that hybridize to the sequence variants of the invention, such as, for instance, nucleotide sequences set forth as SEQ ID NOs: 1-88, 122-308 and 325-374 and fragments thereof, wherein the fragment contains a sequence variation. In other embodiments, the nucleotide sequences are those set forth as SEQ ID NOs: 89-121 and 309-324. In some embodiments, the nucleotide sequences are those set forth as SEQ ID NOs: 53-67, 76, 77, 89, 100, 131-135, 165-175, 253-262, 268, 287, 288, 294, 298-304, 309,331-333 and 341-347.

Another example of a kit includes components such as primers useful for amplification of one or more sequence variants and/or other chemicals for PCR amplification. The primers are constructed and arranged to selectively amplify a region of a nucleic acid molecule that is suspected of containing one or more sequence variations. It is within the skill of the art to construct and arrange primers necessary to assess the genotype of a subject.

The kits provided can also, optionally, contain one or more control agents. The kits also may contain instructions for using the probes/primers of the invention and to correlate the hybridization/amplification to a subject's sensitivity to a therapeutic agent, according to the methods described herein.

In some embodiments the sensitivity to the therapeutic agent is determined by analyzing risk factors in addition to determining the subject's genotype. There are a number of risk factors that can contribute to a subject's response to a therapeutic agent. These include but are not limited to age, gender, race and baseline lung function (e.g. forced expiratory volume at one second (FEV₁)). The methods of determining sensitivity to a therapeutic agent in a subject, therefore, in some embodiments includes assessing one or more risk factors in conjunction with determining the presence or absence of one or more sequence variants from a biological sample obtained from the subject.

In some embodiments, the nucleic acid molecules which contain one or more sequence variations as provided herein and the complementary sequences to which they specifically hybridize thereto are provided as isolated nucleic acid molecules. As used herein the term “isolated nucleic acid molecule” means: (i) amplified in vitro by, for example, polymerase chain reaction (PCR); (ii) recombinantly produced by cloning; (iii) purified, as by cleavage and gel separation; or (iv) synthesized by, for example, chemical synthesis. Isolated nucleic molecules of the invention include DNA, genomic DNA, cDNA, RNA or mRNA. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art.

In another aspect of the invention isolated nucleic acid molecules are provided. These nucleic acid molecules contain one or more sequence variations as described in the Examples. For example, isolated nucleic acid molecules are provided which are selected from the following sequences: SEQ ID NOs: 1-374 and fragments thereof. In other instances the isolated nucleic acid molecules are selected from the following sequences: SEQ ID NOs: 1-88, 122-308 and 325-374 and fragments thereof. In still other instances, the nucleic acid molecules are selected from SEQ ID NOs: 89-121 and 309-324. In yet other instances, the nucleic acid molecules are selected from SEQ ID NOs: 53-67, 76, 77, 89, 100, 131-135, 165-175, 253-262, 268, 287, 288, 294, 298-304, 309, 331-333 and 341-347 and fragments thereof. Fragments of the isolated nucleic acid molecules are provided which include portions of the nucleotide sequences which contain one or more sequence variations as described herein. Fragments, for example, are long enough to assure that the presence or absence of its precise sequence indicates the presence or absence of a sequence variant of interest. The sequence variant of interest can be a single allelic variant or a haplotype. Those of ordinary skill in the art may apply no more than routine procedures to determine if a fragment is of the appropriate size for this purpose. Additionally, the complementary sequences hybridizable to the sequence variants as described above are likewise provided.

Another aspect of the invention provides methods for assessing the sensitivity of a subject to a therapeutic agent by determining the presence or absence of a mutant protein or fragment thereof encoded by a sequence variant described herein. The methods of the invention may also be accomplished using the mutant polypeptides (including whole proteins and partial proteins) that are encoded by the sequence variants described herein. Such mutant polypeptides are useful, for example, alone or as fusion proteins to generate antibodies, and as components of a diagnostic assay. Mutant polypeptides can be isolated from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed mutant protein. Fragments of the mutant polypeptides also can be synthesized chemically using well-established methods of peptide synthesis.

Fragments of a mutant polypeptide preferably are those fragments that retain a distinct functional capability of the mutant polypeptide. Functional capabilities that can be retained in a fragment of a mutant polypeptide include interaction with antibodies or MHC molecules (e.g. immunogenic fragments), interaction with other polypeptides or fragments thereof and selective binding of nucleic acids or proteins. As will be recognized by those skilled in the art, the size of the fragment that can be used for inducing an immune response will depend upon factors such as whether the epitope recognized by an antibody is a linear epitope or a conformational epitope or the particular MHC molecule that binds to and presents the fragment (e.g. HLA class I or II). Thus, some immunogenic fragments of mutant polypeptides will consist of longer segments while others will consist of shorter segments, (e.g. about 5, 6, 7, 8, 9, 10, 11 or 12 or more amino acids long, including each integer up to the full length of the mutant polypeptide). Those skilled in the art are well versed in methods for selecting immunogenic fragments of polypeptides.

The invention, in one aspect, also permits the construction of gene “knock-outs” and “knock-ins” in cells and in animals, providing materials for studying certain aspects of a disease, such as COPD and asthma, therapeutic sensitivity, and immune system responses.

An expression vector comprising any of the isolated nucleic acid molecules preferably operably linked to a promoter may be used to generate the proteins. Host cells transformed or transfected with such expression vectors may also be used. As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids, and virus genomes. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art, e.g., -galactosidase or alkaline phosphatase, and genes which visibly affect the phenotype of transformed or transfected cells, hosts, colonies or plaques, e.g., green fluorescent protein. Preferred vectors are those capable of autonomous replication and expression of the structural gene products present in the DNA segments to which they are operably joined.

As used herein, a coding sequence and regulatory sequences are said to be “operably joined” when they are covalently linked in such a way as to place the expression or transcription of the coding sequence under the influence or control of the regulatory sequences. As used herein, “operably joined” and “operably linked” are used interchangeably and should be construed to have the same meaning. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region is operably joined to a coding sequence if the promoter region is capable of effecting transcription of that DNA sequence such that the resulting transcript can be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Often, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

It will also be recognized that the invention embraces the use of the sequence variants in expression vectors, as well as to transfect host cells and cell lines, be these prokaryotic, e.g., E. coli, or eukaryotic, e.g., CHO cells, COS cells, yeast expression systems, and recombinant baculovirus expression in insect cells. Especially useful are mammalian cells such as human, mouse, hamster, pig, goat, primate, etc. They may be of a wide variety of tissue types, including mast cells, fibroblasts, oocytes, and lymphocytes, and may be primary cells and cell lines. Specific examples include dendritic cells, peripheral blood leukocytes, bone marrow stem cells and embryonic stem cells. The expression vectors require that the pertinent sequence, i.e., those nucleic acids described supra, be operably linked to a promoter.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA or RNA encoding sequence variants or fragments thereof. The heterologous DNA or RNA is placed under operable control of transcriptional elements to permit the expression of the heterologous DNA in the host cell.

Preferred systems for mRNA expression in mammalian cells are those such as pcDNA1.1 and pCDM8 (Invitrogen) that contain a selectable marker (which facilitates the selection of stably transfected cell lines) and contain the human cytomegalovirus (CMV) enhancer-promoter sequences. Additionally, suitable for expression in primate or canine cell lines is the pCEP4 vector (Invitrogen), which contains an Epstein Barr virus (EBV) origin of replication, facilitating the maintenance of plasmid as a multicopy extrachromosomal element. Another expression vector is the pEF-BOS plasmid containing the promoter of polypeptide Elongation Factor 1, which stimulates efficiently transcription in vitro. The plasmid is described by Mizushima and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for E1 and E3 proteins (J. Clin. Invest. 90:626-630, 1992). The use of the adenovirus as an Adeno. P1A recombinant is described by Warnier et al., in intradermal injection in mice for immunization against P1A (Int. J. Cancer, 67:303-310, 1996).

The invention also embraces kits termed “expression kits”, which allow the artisan to prepare a desired expression vector or vectors. Such expression kits include at least separate portions of each of the previously discussed coding sequences. Other components may be added, as desired, as long as the previously mentioned sequences, which are required, are included.

Agents which bind to mutant proteins encoded by sequence variants of the invention, and/or to fragments of the mutant proteins are useful according to the invention. Such binding agents can be used in screening assays to detect the presence or absence of a mutant protein or fragment thereof and in purification protocols to isolate such mutant polypeptides. Likewise, such binding partners can be used to selectively target drugs, diagnostic molecules or other molecules to cells which express the mutant polypeptides. Such binding agents also can be used to inhibit the native activity of the mutant proteins, for example, to further characterize the functions of these molecules. The agents may be polypeptides or other types of molecules that bind to the mutant proteins or fragments thereof. Such binding agents can be used, for example, in screening assays to detect the presence or absence of mutant proteins or fragments thereof and can be used in quantitative binding assays to determine levels of expression in biological samples and cells. Such agents also may be used to inhibit the native activity of the mutant proteins, for example, by binding to the mutant proteins.

According to this aspect, the binding polypeptides bind to an isolated sequence variant or mutant protein of the invention, including fragments thereof. Preferably, the binding polypeptides bind to a mutant protein. Specific binding of the binding polypeptides can be determined by a variety of methods known to those of skill in the art. Such methods include Western blots, dot blots, immunoassays, etc.

The binding polypeptide may be an antibody or antibody fragment, an Fab or F(ab)₂ fragment of an antibody. Typically, the fragment includes a CDR3 region that is selective for the mutant polypeptide. Any of the various types of antibodies can be used for this purpose, including polyclonal antibodies, monoclonal antibodies, humanized antibodies, and chimeric antibodies.

The antibodies may be prepared by any of a variety of methods, including administering a mutant protein, fragments of a mutant protein, cells expressing the mutant protein or fragments thereof and the like to an animal to induce polyclonal antibodies. The present invention also provides methods of producing monoclonal antibodies to the mutant proteins or fragments thereof of the invention described herein. The production of monoclonal antibodies is according to techniques well known in the art. As detailed herein, such antibodies may be used for example to identify tissues expressing mutant protein or to purify mutant protein. Antibodies also may be coupled to specific labeling agents or imaging agents, including, but not limited to a molecule preferably selected from the group consisting of fluorescent, enzyme, radioactive, metallic, biotin, chemiluminescent, bioluminescent, chromophore, or colored, etc. In some aspects of the invention, a label may be a combination of the foregoing molecule types. Agents are coupled to the antibodies or antigen-binding fragments thereof by standard coupling procedures.

These antibody or antigen-binding fragment conjugates can then be used in the methods and kits of the invention. For example, fluorescently labeled or radiolabeled antibody that selectively binds to a mutant polypeptide of the invention may be contacted with a tissue or cell to visualize the mutant polypeptide in vitro or in vivo. Binding can be analyzed with immunologically based assay methods, which include, but are not limited to immunohistochemistry, antibody sandwich capture assay, ELISA, and enzyme-linked immunospot assay (EliSpot assay). These and other in vitro and in vivo imaging methods for determining the presence of the sequence variants and mutant polypeptides of the invention are well known to those of ordinary skill in the art.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of nonspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762, and 5,859,205.

Fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, binding polypeptides of numerous size and type that bind specifically to mutant proteins or fragments thereof are provided. These binding polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptides and non-peptide synthetic moieties.

The mutant proteins or fragments thereof of the invention can be used to screen peptide libraries, including phage display libraries, to identify and select peptide binding partners of the mutant proteins or fragments thereof of the invention. Such molecules can be used, as described, for screening assays, for diagnostic assays, for purification protocols or for targeting drugs, therapeutic agents and/or labeling agents (e.g., radioisotopes, fluorescent molecules, etc.) to cells which express the mutant proteins.

Phage display can be particularly effective in identifying binding peptides useful according to the invention. Briefly, one prepares a phage library (using e.g. m 13, fd, or lambda phage), displaying inserts from 4 to about 80 amino acid residues using conventional procedures. The inserts may represent, for example, a completely degenerate or biased array. One then can select phage-bearing inserts which bind to the mutant proteins or fragments thereof. This process can be repeated through several cycles of reselection of phage that bind to the mutant polypeptide. Repeated rounds lead to enrichment of phage bearing particular sequences. DNA sequence analysis can be conducted to identify the sequences of the expressed polypeptides. The minimal linear portion of the sequence that binds to the mutant polypeptide can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to the mutant polypeptides.

Diagnostic agents for in vivo use include, but are not limited to, barium sulfate, iocetamic acid, iopanoic acid, ipodate calcium, diatrizoate sodium, diatrizoate meglumine, metrizamide, tyropanoate sodium and radiodiagnostics including positron emitters such as fluorine-18 and carbon-11, gamma emitters such as iodine-123, technitium-99, iodine-131 and indium-11, and nuclides for nuclear magnetic resonance such as fluorine and gadolinium. Other diagnostic agents useful in the invention will be apparent to one of ordinary skill in the art.

The invention also includes kits for assaying the presence of mutant proteins or fragments thereof. An example of such a kit may include the above-mentioned binding polypeptides bound to a substrate, for example a dipstick, which is dipped into a blood or body fluid sample of a subject. The surface of the substrate may then be processed using procedures well known to those of skill in the art, to assess whether specific binding occurred between the mutant polypeptides and agents (e.g. antibodies) in the subject's sample. For example, procedures may include, but are not limited to, contact with a secondary antibody, or other method that indicates the presence of specific binding.

Another example of a kit may include an antibody or antigen-binding fragment thereof, that binds specifically to a mutant protein or fragment thereof. The antibody or antigen-binding fragment thereof, may be applied to a tissue or cell sample from a subject, and the sample then processed to assess whether specific binding occurs between the antibody and mutant polypeptide of the sample. In addition, the antibody or antigen-binding fragment thereof, may be applied to a body fluid sample, such as serum, from a subject, either suspected of having or diagnosed with a disease, such as COPD or asthma. As will be understood by one of skill in the art, such binding assays may also be performed with a sample or object contacted with an antibody and/or mutant protein or fragment thereof that is in solution, for example in a 96-well plate or applied directly to an object surface.

The foregoing kits can include instructions or other printed material on how to use the various components of the kits for diagnostic purposes. More specifically instructions are provided to correlate the presence or absence of the sequence variants or mutant proteins or fragments thereof as described herein with a subject's sensitivity to a therapeutic agent.

The invention further includes protein microarrays (including antibody arrays) for the analysis of expression of mutant polypeptides. In this aspect of the invention, standard techniques of microarray technology are utilized to assess expression of the mutant polypeptides and/or identify biological constituents that bind such mutant polypeptides. The constituents of biological samples include antibodies, lymphocytes (particularly T lymphocytes), and the like. Microarray substrates include but are not limited to glass, silica, aluminosilicates, borosilicates, metal oxides such as alumina and nickel oxide, various clays, nitrocellulose, or nylon. The microarray substrates may be coated with a compound to enhance synthesis of a peptide probe on the substrate. Coupling agents or groups on the substrate can be used to covalently link the first amino acid to the substrate. A variety of coupling agents or groups are known to those of skill in the art. Peptide probes thus can be synthesized directly on the substrate in a predetermined grid. Alternatively, peptide probes can be spotted on the substrate, and in such cases the substrate may be coated with a compound to enhance binding of the probe to the substrate. In these embodiments, presynthesized probes are applied to the substrate in a precise, predetermined volume and grid pattern, preferably utilizing a computer-controlled robot to apply probe to the substrate in a contact-printing manner or in a non-contact manner such as ink jet or piezo-electric delivery. Probes may be covalently linked to the substrate.

Protein microarray technology, which is also known by other names including protein chip technology and solid-phase protein array technology, is well known to those of ordinary skill in the art and is based on, but not limited to, obtaining an array of identified peptides or proteins on a fixed substrate, binding target molecules or biological constituents to the peptides, and evaluating such binding. See, e.g., G. MacBeath and S. L. Schreiber, “Printing Proteins as Microarrays for High-Throughput Function Determination,” Science 289(5485):1760-1763, 2000.

Targets are peptides or proteins and may be natural or synthetic. The tissue may be obtained from a subject or may be grown in culture (e.g. from a cell line).

In some embodiments of the invention, one or more control peptide or protein molecules are attached to the substrate. Preferably, control peptide or protein molecules allow determination of factors such as peptide or protein quality and binding characteristics, reagent quality and effectiveness, hybridization success, and analysis thresholds and success.

### EXAMPLES

### Example 1

### Initial Population Studies and Genotyping

Materials and Methods

Test Populations

DNA and phenotypic information (bronchodilator response and steroid reponse) available for the participants from two large clinical trials were utilized to test our hypothesis on candidate genes. The Forest study (also known as the Adult Study) was an 8 week clinical trial used to compare the effect of once daily high dose inhaled flunisolide vs. “standard inhaled corticosteroid therapy” (performed by Forest Pharmaceuticals). 470 participants had an average of 7.0% improvement in their forced expiratory volume at one second (FEV₁) at the end of the trial period (mean FEV₁ at enrollment was 72%). Of these participants, 7% were African American, 3% were Hispanic and 2% were other. As 88% of these participants were Caucasian, analyses focus on this group. Additionally, females accounted for 58.5% of the participants.

The Childhood Asthma Management Program (CAMP) was a randomized, double-masked, long term clinical trial testing the safety and efficacy of inhaled budesonide vs. nedocromil vs. placebo (27). The mean duration of follow-up was 4 years (28). 311 children (males and females for a variety of race and age groups) were randomized to the steroid group, with a mean improvement of 6.8% in their FEV₁ at one year. Of these, 65% were Caucasian. These formed the basis for our replication sample.

Sequencing

Novel SNPs were obtained by screening 24 Coriel and 14 asthma cell lines using an ABI 3700 Sequencer (Applied Biosystems, Foster City, Calif.).

Genotyping

Genotyping of the SNPs was performed primarily via utilization of a SEQUENOM MassARRAY Matrix Assisted Laser Desorption and Ionization Time of Flight (MALDI-TOF) mass spectrometer (Sequenom, San Diego, Calif.) for analysis of unlabeled single-base extension minisequencing reactions. For the MALDI-TOF system, a semi-automated primer design program was utilized (Spectro DESIGNER, Sequenom). Our protocol implemented the very short extension method (VSET) proposed by Sun and colleagues (33), whereby sequencing products were extended by only one base for 3 of 4 nucleotides in the minisequencing reactions and by several additional bases for the fourth nucleotide which was specified in advance to represent one of the two alleles at a given SNP locus. This allowed for mass separation of the two allelic variants at a given locus by at least several hundred kDa.

Analytic Methods

Initial analysis was performed using single alleleic χ2 tests (dichotomous, high versus low response outcomes) and ANOVA (continuous outcomes), as well as screening Clump, a permutation based χ2 exact test, for haplotype analysis (30). Contingency tables were then constructed to show the haplotype specific odds ratio after analysis using Clump.

Haplotype Imputation

Phase (29) was run on both the entire populations and on the high and low response groups for the dichotomous outcomes for each gene. Minor modifications to increase availability and efficiency of Phase were performed. Output for the whole populations from Phase were then used in a haplotype-tagging program (BEST) (32). Haplotype-tagged output was utilized for analyses involving continuous outcomes and covariate adjustments. Contingency tables were also constructed to compare side by side haplotype frequencies and imputed counts for each categorical outcome.

Haplo.score Analysis

Utilizing the haplotype-tagged SNPs, this expectation maximization (EM) algorithm based program allowed for analysis of both continuous and categorical phenotypes, with and without covariate adjustment. Modifications of this program allowed for imputation of missing values to increase power for analysis, utilizing both an EM- and Phase-based approach to estimate missingness. From this analysis, common risk haplotypes were identified.

Results

The initial candidate genes were chosen by a panel of asthma and endocrine specialists and included genes involved in innate glucocorticoid synthesis and metabolism, as well as genes crucial as receptors and transcriptional regulators of corticosteroids. Table 1 presented below provides a list of these genes as well as their general function in the glucocorticoid pathway.

TABLE 1
List of Initial Pathway Candidate Genes

| Gene | Glucocorticoid Effect |
|---|---|
| CRH | Genesis of Steroid |
| CRHR1 | Genesis of Steroid |
| ACTH (POMC) | Genesis of Steroid |
| CRHBP | Receptor Binding |
| NR3C1 (GRL) | Receptor Binding |
| GATA3 | Transcriptional Regulation |
| MAPK8 | Transcriptional Regulation |
| NFATC4 | Transcriptional Regulation |
| EGR1 | Immediate Downstream Mediation |
| STAT3 | Immediate Downstream Mediation |
| STAT5A | Immediate Downstream Mediation |
| STAT6 | Immediate Downstream Mediation |
| TGFbeta | Immediate Downstream Mediation |
| ALOX15 | Pathway Interactions |
| Eotaxin (SCYA11) | Pathway Interactions |
| FCER2 (CD23) | Pathway Interactions |
| IL18BP | Pathway Interactions |
| TBET (TBX21) | Pathway Interactions |
| HSD11B1 | Steroid Metabolism |
| HSD11B2 | Steroid Metabolism |

Sequence variants for subsequent genotyping in the Forest group for the genes listed in Table 1 were determined from public databases as well as sequencing of Coriel and asthma cell lines. The Forest samples were genotyped and a subset of the genes were replicated within the Camp samples. Based on the analyses of the single allelic variant effects, the following table (Table 2) provides a summary list of the polymorphisms identified that are associated with the response phenotype.

TABLE 2

Summary List of Polymorphisms and Flanking Sequences

| | | |
|---|---|---|
| Gene | SNP | Polymorphism and Flanking Sequences |

| | | | |
|---|---|---|---|
| ALOX15 | rs1318629 | ACGCAGGAAGAGGGAATCAACGCCTGGTACAGCAGGCAGGCGAGG | (SEQ ID NO: 1) |
| | | TGGGGGTAGGAGTTATGCACGTGTGTACCACGGACTTTGGGCCAAG | |
| | | CCTGGGTGG[C/T]TGGGAAGCCAACCTCCATCTAAACGCACGCGTGC | |
| | | ACACACACACACACGCAAGGACACGCGCGCGCACACACAAGCCTCA | |
| | | CAAGTTGGATTGCAGGAGAG | |
| ALOX15 | rs1871346 | ATCATCTGGTTACAAGTATTCTCAGCTGAAAATCATTTTCACACAGA | (SEQ ID NO: 2) |
| | | AGCTTGAAGGCATTGTTTTCTTTCAGTGTGGCTATTTAGAAGTCCAA | |
| | | GGCTGA[C/T]ACCTGATCTTTCGTATGTTTTTCTCTCTCAGGGAGCTTT | |
| | | AGAAGTCTATTCTTTATTCTGGGTATTCTGAAATTTGTGATGATGCAC | |
| | | CTTGGGGTGGGCA | |
| ALOX15 | rs1904304 | GAGGTCAGGCATTCGAGACCAGCCTGGCCAACATGGTGAAATCCTG | (SEQ ID NO: 3) |
| | | T[C/T]TCTATTAAAAATACAAAAAAAATTAGCCAGGCCTGGTGGTGC | |
| | | GCGACTGTAATCCCAGCTACTGGGGAGGCTGAAGTGGGAGAATTGC | |
| | | TTAAACCCAGGAGGCAGAGGTTGCAGTGAGCCGAGATCGCATCACT | |
| | | GCACTCCAGTCTGGGGAAAGGGAGACTCCATCTCAAAAAAAAAAAA | |
| | | AAAATCAATGTCTGGAAAAGATCTTTTTAACGTTATTTCACGGGTTC | |
| | | CTCTTGTTGGATGGAATCCTTGGTCATTTGCATGTACTGTGCTTTACA | |
| | | TATAAAAAGAGTAAGATTATTTTCGCCACTTTCCCAGGTGGGAGGTG | |
| | | CTATCCCCTTTGAAAATGCATGGCCAGCCCTGCTCATTCTCTGTTGCT | |
| | | CTCACAT | |
| ALOX15 | rs1965923 | AAAAACTACCTATGGGGTACCATGCTTATTACCTGGTGACAAAATAA | (SEQ ID NO: 4) |
| | | TCTGTACACCAAGCCCCATGACACACAATTTACTTACAGAACAAACC | |
| | | TGCCCATGTATCCTTGAACCTAAAATTAAAGTTAAAAATAAATACAT | |
| | | AAAAATAAAACCACTGAGCTTTCAGTTGATTTGCATCAACTATTTCC | |
| | | ATTCCACAAACCAGACTTAGTGCTTTATGATAATTGAAAGAAATCAT | |
| | | TCTTTGTTGTTGTTGTTTTGTTTTGAGATGGAGTCTTGCTCTGTTTC[C/ | |
| | | G]GAGGCTGAAGTACAGTGGTGTGATCTCGGCTCACTGCAACCTCCG | |
| | | TCTTCTGGGTTCACGCGATTCTCCTGCCTCAGCCTCCCGAGTAGCTGG | |
| | | GATTACAGGCGTGAACCACCGTGCCTGGCCTCCTTTTCTTTTCATATT | |
| | | TTTTCATAGGCCTAGACCAAGATTTCTCAACTTTTTTTTTTTTAATCA | |
| | | CCCACTCAATACCCCTTAAGAGATGTCTTTTCCTAAACCTCACTCCAC | |
| | | CTATTAAATATTAATGCTACATATATCCTGGATATCTTGCTATG | |
| ALOX15 | rs2077005 | AGAGCGGCAGGAATGCTCAGTTAACTCTTACAGGAATTACCATGAC | (SEQ ID NO: 5) |
| | | AGCAGGTTGGGGTGGGGGAGTGGGGAGCTCTGCCGGGAGGGTCCCA | |
| | | CTCAGTGG[G/T]GTGCTGCTGCTGACTCACACTGGCTCGCAAGAGTC | |
| | | AGTCCATGACGTTAGGTGTTTAGCAAGCTGGTCAGCATCACCTTGGT | |
| | | AGTCACAGTAAAAATATT | |
| ALOX15 | rs2255888 | GGCCACGGAATTCATTCGACCAGCTTTGCACACTTGCCTTCTCTGTG | (SEQ ID NO: 6) |
| | | CCAGCCACTGGGACTCATTCCTTTTGAGCATAAAAGCCGCTGCCTCC | |
| | | CTGTTG[C/T]CTGCAGAATAAAAGTCCAAATGTTTCTGGCATTCAATG | |
| | | CCCTTGCTAACCTGACCTGCTCTCCTCACCTCTCATCCCACTGCACCC | |
| | | TGGCATCTCCTGTG | |
| ALOX15 | rs2619115 | GCCAGCCCTGCTCATTCTCTGTTGCTCTCACATTGTCCATTCATTGTT | (SEQ ID NO: 7) |
| | | TGTCTCTGTACCCCGAGACCCCACAGAGAGGGAGCCTCCAGAGGTC | |
| | | GGAACG[A/T]GCCCTGGGGCAGAGATAGTGGCAGGCAAGAGAGAAG | |
| | | GGGATAAGGAGTTACCTTGAAGATAGGATGTATCGACGGCAGGCAC | |
| | | CTCATGGTGGCCACAACA | |
| ALOX15 | rs2856419 | CCAGAAAATCCGGTTGAAGTCATCTAGATCCTTCCAGCAAGTCAGAA | (SEQ ID NO: 8) |
| | | CATTTAGAGAGTCTTTGATAGCGAGGTCGGCCAGCCTTCAGGGCAGG | |
| | | ATGGGG[C/G]AAAGGGTTTGAGCATCATTCTGAGGCTCTAACATGAC | |
| | | GACCACAGGCACCGATCCTGGGCCAGTCCAATGCAGTGCAGCCCAT | |
| | | AAGCCCCTTGGCTTCCA | |
| ALOX15 | rs743646 | CTGCTCAGCAAGCTGGAGCCTTCCTAACCTACAGCTCCTTCTGTCCC | (SEQ IDNO: 9) |
| | | CCTGATGACTTGGCCGACCGGGGGCTCCTGGGAGTGAAGTCTTCCTT | |
| | | CTATGCCCAAGATGCGCTGCGGCTCTGGGAAATCATCTATCGGTGAG | |
| | | GCAAGCGGGAAGGCCAGTGGGGGTGCAAGTGGGGGTGGAGAAGAC | |
| | | ATGTAGGAGAGCAGGAGGTCTGCGTCTGGTTGGGGGCCTGGGGCCC | |
| | | TGACCTGGCCATGTGAGCAGGGGCAGAGCTGGCTTCAGCTCCCTGGC | |
| | | CCTGCTCCGTTGGTTGGTAGGTATGTGGAAGGAATCGTGAGTCTCCA | |
| | | CTATAAGAC[A/G]GACGTGGCTGTGAAAGACGACCCAGAGCTGCAG | |
| | | ACCTGGTGTCGAGAGATCACTGAAATCGGGCTGCAAGGGGCCCAGG | |
| | | ACCGAGGTAAGAGGAGCCCCTGCCCTGAGATCTCAGACACAAAGCC | |
| | | CAAGAGATCTTCCCAGAATCCCCTGTGCTTCTGTGAAATCTCCCAGA | |
| | | AGCATTTTCAACACCTATGAGAACTCCAGAGGCCTTCTCAGATTCCA | |
| | | CTCCCTGTCACCTAGAGACAGGTCCCCCGTCCTACACACTGAGAACC | |
| | | TCTAGGTGCCAGATGCAGCGGGACCAGTGGCTGCTCATAAATGTTTA | |
| | | ACAACTGACTCTCAGGAGAACGTCCTGATTTGTAGCTTTTGCACATT | |
| | | TCCATGGCTAAATTTTTTTACTGGGACTACAAGGGGGTGCTGAACAG | |
| | | CTTGCTAACACCTACGTTATGGACTGACTTTTGCGAGCCAGTGTGAG | |
| | | TC | |
| ALOX15 | rs762743 | GCTCAAACCCTTTCCCCCATCCTGCCCTGAAGGCTGGCCGACCTCGC | (SEQ ID NO: 10) |
| | | TATCAAAGACTCTCTAAATGTTCTGACTTGCTGGAAGGATCTAGATG | |
| | | ACTTCAACCGGATTTTCTGGTGTGGTCAGAGCAAGCTGGCTGGTCAG | |
| | | TGCCCGACCCCAGTATGTCTCCCAACCCCCCAGATCCCACCCAGATC | |
| | | CCACCCAACCCAGGGGAATTGAAAGA[A/T]GCAGGGTGGGGAGACC | |
| | | AGAGACTTGGGTCCTCTGGTGGGCTGGAGTAAGGGGGCATGGTTGG | |
| | | TGGGGTTGGAAGGACCAAGAGCTCAGATCCCACAACTTGCTCAACA | |
| | | ACTGCCTTTCCCCAGAGCGCGTGCGGGACTCCTGGAAGGAAGATGC | |
| | | CTTATTTGGGTACCAGTTTCTTAATGGCGCCAACCCCGTGGTGCTGA | |
| | | GGCGCTCTGCTCACCTTCCTGCTCGCCTAGTGTTCCCTCCAGGCATGG | |
| | | AGGAACTGCAGGCCC | |
| ALOX15 | rs899452 | AAGACAAGAGAGTTGACTTTGAGGTTTCGCTGGCCAAGGGGTGAGA | (SEQ ID NO: 11) |
| | | GCAAGGGGAGGCTGGGTGAGAGGGAGGTGTCCTGGTCTAGTGGAAG | |
| | | CCAAGGGGCTTATGGGCTGCACTGCATTGGACTGGCCCAGGATCGGT | |
| | | GCCTGTGGTCGTCATGTTAGAGCCTCAGAATGATGCTCAAACCCTTT | |
| | | GCCCCATCCTGCCCTGAAGGCTGGCCGACCTCGCTATCAAAGACTCT | |
| | | CTAAATGTTCTGACTTGCTGGAAGGATCTAGATGACTTCAACCGGAT | |
| | | TTTCTGGTGTGGTCAGAGCAAGCTGGCTGGTCAGTCCCCCACCCCAG | |
| | | TATGTCTCCCAACCCCCCAGATCCCACCCAGATCCCACCCAACCCAG | |
| | | GGGAATTGAAAGAAGCAGGGTGGGGAGACCAGAGACTTGGGTCCCT | |
| | | CTGGTGGGCTGGAGTCAAGG[A/G]GGCATGGTTGGTGGGGTTGGAAG | |
| | | GACCAAGAGCTCAGATCCCACAACTTGCTCAACAACTGCCTTCCCCA | |
| | | GAGCGCGTGCGGGACTCCTGGAAGGAAGATGCCTTATTTGGGTACC | |
| | | AGTTTCTTAATGGCGCCAACCCCGTGGTGCTGAGGCGCTCTGCTCAC | |
| | | CTTCCTGCTCGCCTAGTGTTCCCTCCAGGCATGGAG | |
| ALOX15 | rs916055 | TGTGGGGACGTGTGGCATCCCAGACTGGGGGTCATAAGGCTCTCAG | (SEQ ID NO: 12) |
| | | CCACCTTTTCCTCTCCCTCCCAGGTGGCTGTGGGCCAGCATGAGGAG | |
| | | GAGTATTTTTCGGGCCCTGAGCCTAAGGCTGTGCTGAAGAAGTTCAG | |
| | | GGAGGAGCTGGCTGCCCTGGATAAGGAAATTGAGATCCGGAATGCA | |
| | | AAGCTGGACATGCCCTACGAGTACCTGCGGCCCAGCGTGGTGGAAA | |
| | | ACAGTGTGGCCATCTAAGCGTCGCCACCCTTTGGTTATTTCAGCCCC | |
| | | CATCACCCAAGCCACAAGCTGACCCCTTCG[C/T]GGTTATAGCCCTGC | |
| | | CCTCCCAAGTCCCACCCTCTTCCCATGTCCCACCCTCCCTAGAGGGG | |
| | | CACCTTTTCATGGTCTCTGCACCCAGTGAACACATTTTACTCTAGAG | |
| | | GCATCACCTGGGACCTTACTCCTCTTTCCTTCCTTCCTCCTTTCCTATC | |
| | | TTCCTTGCTCTCTCTCTTCCTCTTTCTTCATTCAGATCTATATGGCAAA | |
| | | TAGCCACAATTATATAAATCATTTCAAGACTAGAATAGGGGGATATA | |
| | | ATACATATTACTCCACACCTT | |
| ALOX15 | rs916056 | TGTGGGGACGTGTGGCATCCCAGACTGGGGGTCATAAGGCTCTCAG | (SEQ ID NO: 13) |
| | | CCACCTTTTCCTCTCCCTCCCAGGTGGCTGTGGGCCAGCATGAGGAG | |
| | | GAGTATTTTTCGGGCCCTGAGCCTAAGGCTGTGCTGAAGAAGTTCAG | |
| | | GGAGGAGCTGGCTGCCCTGGATAAGGAAATTGAGATCCGGAATGCA | |
| | | AAGCTGGACATGCCCTACGAGTACCTGCGGCCCAGCGTGGTGGAAA | |
| | | ACAGTGTGGCCATCTAAGCGTCGCCACCCTTTGGTTATTTCAGCCCC | |
| | | CATCACCCAAGCCACAAGCTGACCCCTTCGTGGTTATAGCCCTGCCC | |
| | | TCCCAAGTCCCACCCTCTTCCCATGTCCCACCCTCCCTAGAGGGGCA | |
| | | CCTTTTCATGGTCTCTGCACCCAGTGAACACATTTTACTCTAGAGGC | |
| | | ATCACCTGGGACCTTACTCCTCTTTCCTTCCTTCCTCCTTTCCTATCTT | |
| | | CCTT[C/G]CTCTCTCTCTTCCTCTTTCTTCATTCAGATCTATATGGCAA | |
| | | ATAGCCACAATTATATAAATCATTTCAAGACTAGAATAGGGGGATAT | |
| | | AATACATATTACTCCACACCTT | |
| CRH | rs1396862 | TGGAATGTATGCCCTACGCCAGGCCCATGGAATCGGAGCTTGGTTTT | (SEQ ID NO: 14) |
| Receptor | | AGGAAAAAGCACCT[C/T]TGCAGTTCAGAAGCCCTGGTCCAACCACC | |
| (CRHR1) | | ACTCACCTCTCCCCACACGGTGAGCAGTGAACCTTGGTCCACAAACC | |
| | | AGACCCCCAGAATCCGTTTCATGTCCCACCACGGTGTGCTCTCCCAG | |
| | | GCTGTGCCTGAGGCCCAGGCTCCTGTGCGCAGCAGAACGTGGGGAA | |
| | | AGGGGATAGAGTGTGTGCAAGTGTTGGGTGGGTGGGGGAAGGCTGG | |
| | | ACGGTGGGGAAGGGAGTGAACAGTAGTAGCGGGAGGTGGTGGGGG | |
| | | GAGGAGAGGGTGCTGATACAGGCAGCAGGTGTAGGGGTGGTGCTGG | |
| | | GGGCTCAGTGCCATCCCCCGGCCAGCCATGCATGCAACAGTTGGGG | |
| | | GCCTGTCCCCTGGT | |
| CRH | rs171440 | TGCAGAAATGAGCTCATCCTATGCAAAGAAGACACAAGGGGAAGAA | (SEQ ID NO: 15) |
| Receptor | | ATCTGAATCTGATGCTATGGTACTAGGGAGCCAGCATGGGTTCCAGA | |
| (CRHR1) | | GCAGAGGAGGAAGAGAGATGGCTGGAGGTGGTGTTAAAAGCTAAG | |
| | | GTCCTGGCATTTAGAGGAAGCCTGGGCATGCTTCTGGTCCCCTGCTC | |
| | | TGTAGCCTAAGGACA[C/T]TTCTCTTGGTCCCTCGCATGGTGACAGCC | |
| | | TGGAGCTCTGAGACATCACAGGAACACCCTGGAACAGACCCATCCA | |
| | | ATCATTGACCTAGCCCCTCACGTCTCTGCAATCAAACCACATCTACC | |
| | | CTGGCCCTGCAGGGAAGAATCAGCTAAATGAAGTTGGCCCTCCTTCC | |
| | | GGCTGGCCTGTTCCTTCTTCCGGCTGGCCTG | |
| CRH | rs171441 | TGGCATTTAGAGGAAGCCTGGGCATGCTTCTGGTCCCCTGCTCTGTA | (SEQ ID NO: 16) |
| Receptor | | GCCTAAGGACATTTCTCTTGGTCCCTCGCATGGTGACAGCCTGGAGC | |
| (CRHR1) | | TCTGAGACATCACAGGAACACCCTGGAACAGACCCATCCAATCATT | |
| | | GACCTAGCCCCTCACGTCTCTGCAATCAAACCACATCTACCCTGGCC | |
| | | CTGCAGGGAAGAA[C/T]CAGCTAAATGAAGTTGGCCCTCCTTCCGGC | |
| | | TGGCCTGTTCCTTCTTCCGGCTGGCCTGTTAACCACACTTACTGACCA | |
| | | TACGTCACGTGTACTTTGTTCTGTGCAGGGCCCTAGGACAGGACTGA | |
| | | TGGAGAAGTGAGCTATGGAGAGGAAAGGGAGGGAGACACCATCTG | |
| | | GGGGAGTGGAAAGGACACACTGAACTGGGAGTCTGGGCCCACTGCT | |
| | | TCAGAATGGGCTGTGCCACTAATTTGCTGTGAGATCATAGGCAAGTC | |
| | | ACTTGCCCTCTCTGGGCCTTAGTGTCTTGTCTTCATCTATAATAAAAT | |
| | | CAAGTGGTTGAACCAGATCAGAAATTCTTAATCTCAGCCCACTGGAA | |
| | | ACTTCAACAGTTCACAGAGACCCTGAATGTAATGAAAATATTGGTGT | |
| CRH | rs171442 | TCTGTGAATGTTATCACGGAGTGGCAGTGGAAGCAGTGGAGTGAGG | (SEQ ID NO: 17) |
| Receptor | | AAGCAGGGCCATCCGAGGCAACGGCGTACCAGGCTGGGATTTTAAC | |
| (CRHR1) | | CTGTCCTGGATCCTGCTGACAGTGTTGAGCCCGGGGACAGGGACTTG | |
| | | GGGGGAGAGCTGGGGGCATGTGGCCGCGGGGAGGGGAGGATCGTA | |
| | | TAGAGGCCATGCCCTGCTGTTTCAAAGCTCTTCATCTATTTCGTTTGA | |
| | | TCTTCA[C/T]ACATCTGGCAATGTGATTATTTCCTTTCTACAGATGAA | |
| | | GAAATTGAGGCCCAGGGAAGTTAGGTGACTTTTCCAAGGTCATGGG | |
| | | TCAATGGGTGAAAGAACCAAGACTCATAGGCAGGTATTTTGGAGTCT | |
| | | AAATCTAGTGCTCTTTCCATTACGCCACATTGTGAGTCAGTCACAGG | |
| | | GGGTGAGGGACAGTTAAGGGGGCAGCAGGAAGGGAGCAGCGTTTGT | |
| | | TGAGGGCTGACTGTGTGCCAGGCACACATGACATATGCCATCTCATT | |
| | | TGATGGCACCATTGTACCTTCCTCAGTAGCTTGCTCATTCACTCCTCA | |
| | | CTCATTCATCCACAGTATCTAGGGAGCTAGGCTCAAATGTAATGTTC | |
| | | CATGTGGCAGCGTCTGGAATGAACACACGGAACACACCCTCTCCATC | |
| | | AGTGTGGCCCCT | |
| CRH | rs173365 | GCCCCTCGCCCTGCCAGAGCAGCACCGTGGAAGAACTCGTGAGTCTT | (SEQ ID NO: 18) |
| Receptor | | TTAAGCTAGGCATTGACCTAGCTGCAGCTTCCGGAAGGAGACAGCA | |
| (CRHR1) | | GAGCCCCGCATTAGCTCAGTGACTTGAGGCCAGGAAGCCAGGGGTG | |
| | | GGGCTAACCAAAGCTTGCCAGGCCGGGGTGGGAGCTACAGGTGAAG | |
| | | GAAAGTGATTCTTTC[C/T]CCGTTAACTTTGTTTCACGCCAGATACCT | |
| | | GGCAGTGGGCAAAGCAGAGCC | |
| CRH | rs1876827 | GTACATGGAAGTCCTCAGTAGGGGATGACACTCACAGCCTTAACAC | (SEQ ID NO: 19) |
| Receptor | | G[A/G]CTGCTTTGCATATTTGTCGGAACAGGTTTCTCAAATGTCCTGG | |
| (CRHR1) | | GGAAGGGGCACCCTTTTCTAACCCACACGAAGGCACCATATGCCCTT | |
| | | TGCCATGAAGGCTCCCTGCCCTCAACCCACATGTATCCCCTGCCCAG | |
| | | GGCTCAGCCCTTCCTGGTTTTCACAGGCTCATCAAAGATACTGGAGC | |
| | | TCTGGCTTCCAGCCTGGTGCCAGCGGCCTCTGAGAGCAAAGGAGGG | |
| | | GGCTGTCACTGTGGGAGTGGACAGGTGGGAGGGGCCACCCTGGGGC | |
| | | TCCCAGCAGCATACCCCTAGGGACCTAGGAGCAGGGAGGGAGAGAG | |
| | | GCAGCCCTGGGAGGGGAGGAGAAGGCTCTAGACAATCGCCAGTCCC | |
| | | AACAGGCCTCACAGCCCTGAACCCCGCTGCAGGGCCCCCGGGTCCTC | |
| | | ACCTCACTATTGAGGAAACAGTAGAACACAGACACAAAGAAGCCCT | |
| | | GGGGAGGAGAGAGGAGGCGTCAAAGGTCGGCTGCAGGTGTTGCCCA | |
| | | CCCAGCCTCTGGGCTGCCCCTTGCTTCTCCCTGGCCTCCTGCCCTCCA | |
| | | GGCCTCTGCTTGGCAGAATCCCCACC | |
| CRH | rs1876828 | GTACATGGAAGTCCTCAGTAGGGGATGACACTCACAGCCTTAACAC | (SEQ ID NO: 20) |
| Receptor | | GACTGCTTTGCATATTTGTCGGAACAGGTTTCTCAAATGTCCTGGGG | |
| (CRHR1) | | AAGGGGCACCCTTTTCTAACCCACACGAAGGCACCATATGCCCTTTG | |
| | | CCATGAAGGCTCCCTGCCCTCAACCCACATGTATCCCCTGCCCAGGG | |
| | | CTCAGCCCTTCCTGGTTTTCACAGGCTCATCAAAGATACTGGAGCTC | |
| | | TGGCTTCCAGCCTGGTGCCAGCGGCCTCTGAGAGCAAAGGAGGGGG | |
| | | CTGTCACTGTGGGAGTGGACAGGTGGGAGGGGCCACCCTGGGGCTC | |
| | | CCAGCAGCATACCCCTAGGGACCTAGGA[A/G]CAGGGAGGGAGAGA | |
| | | GGCAGCCCTGGGAGGGGAGGAGAAGGCTCTAGACAATCGCCAGTCC | |
| | | CAACAGGCCTCACAGCCCTGAACCCCGCTGCAGGGCCCCCGGGTCCT | |
| | | CACCTCACTATTGAGGAAACAGTAGAACACAGACACAAAGAAGCCC | |
| | | TGGGGAGGAGAGAGGAGGCGTCAAAGGTCGGCTGCAGGTGTTGCCC | |
| | | ACCCAGCCTCTGGGCTGCCCCTTGCTTCTCCCTGGCCTCCTGCCCTCC | |
| | | AGGCCTCTGCTTGGCAGAATCCCCACC | |
| CRH | rs1876829 | GTACATGGAAGTCCTCAGTAGGGGATGACACTCACAGCCTTAACAC | (SEQ ID NO: 21) |
| Receptor | | GACTGCTTTGCATATTTGTCGGAACAGGTTTCTCAAATGTCCTGGGG | |
| (CRHR1) | | AAGGGGCACCCTTTTCTAACCCACACGAAGGCACCATATGCCCTTTG | |
| | | CCATGAAGGCTCCCTGCCCTCAACCCACATGTATCCCCTGCCCAGGG | |
| | | CTCAGCCCTTCCTGGTTTTCACAGGCTCATCAAAGATACTGGAGCTC | |
| | | TGGCTTCCAGCCTGGTGCCAGCGGCCTCTGAGAGCAAAGGAGGGGG | |
| | | CTGTCACTGTGGGAGTGGACAGGTGGGAGGGGCCACCCTGGGGCTC | |
| | | CCAGCAGCATACCCCTAGGGACCTAGGAGCAGGGAGGGAGAGAGG | |
| | | CAGCCCTGGGAGGGGAGGAGAAGGCTCTAGACAATCGCCAGTCCCA | |
| | | ACAGGCCTCACAGCCCTGA[A/G]CCCCGCTGCAGGGCCCCCGGGTCC | |
| | | TCACCTCACTATTGAGGAAACAGTAGAACACAGACACAAAGAAGCC | |
| | | CTGGGGAGGAGAGAGGAGGCGTCAAAGGTCGGCTGCAGGTGTTGCC | |
| | | CACCCAGCCTCTGGGCTGCCCCTTGCTTCTCCCTGGCCTCCTGCCCTC | |
| | | CAGGCCTCTGCTTGGCAGAATCCCCACC | |
| CRH | rs1876830 | GTACATGGAAGTCCTCAGTAGGGGATGACACTCACAGCCTTAACAC | (SEQ ID NO: 22) |
| Receptor | | GACTGCTTTGCATATTTGTCGGAACAGGTTTCTCAAATGTCCTGGGG | |
| (CRHR1) | | AAGGGGCACCCTTTTCTAACCCACACGAAGGCACCATATGCCCTTTG | |
| | | CCATGAAGGCTCCCTGCCCTCAACCCACATGTATCCCCTGCCCAGGG | |
| | | CTCAGCCCTTCCTGGTTTTCACAGGCTCATCAAAGATACTGGAGCTC | |
| | | TGGCTTCCAGCCTGGTGCCAGCGGCCTCTGAGAGCAAAGGAGGGGG | |
| | | CTGTCACTGTGGGAGTGGACAGGTGGGAGGGGCCACCCTGGGGCTC | |
| | | CCAGCAGCATACCCCTAGGGACCTAGGAGCAGGGAGGGAGAGAGG | |
| | | CAGCCCTGGGAGGGGAGGAGAAGGCTCTAGACAATCGCCAGTCCCA | |
| | | ACAGGCCTCACAGCCCTGAACCCCGCTGCAGGGCCCCCGGGTCCTCA | |
| | | CCTCACTATTGAGGAAACAGTAGAACACAGACACAAAGAAGCCCTG | |
| | | GGGAGGAGAGAGGAGGC[A/G]TCAAAGGTCGGCTGCAGGTGTTGCC | |
| | | CACCCAGCCTCTGGGCTGCCCCTTGCTTCTCCCTGGCCTCCTGCCCTC | |
| | | CAGGGCTCTGCTTGGCAGAATCCCCACC | |
| CRH | rs1876831 | TGTAGGCGGCTGTCACCAACCTGCACCAGCCCTGCCACCCCACCCCC | (SEQ ID NO: 23) |
| Receptor | | AACCAGAGATGATGATGGGGG[A/G]CAGGGGAGGCACCAAACCCTG | |
| (CRHR1) | | GGCCTGGGCCTCCCCAGGGCAGGACAGGGCATACCCTGGGATCCCA | |
| | | CTCCTGTTCTGTGGGCTCCTCCTCTGCAGGGCAGGCGGGCCCCTCCT | |
| | | CTGACCTGGGTTTGGCCTGACCTGCTCCCCGCTCCCCTGCCAGGACG | |
| | | TACCACGTTGCTCTGGTGGACCTCGGGGCTCATGGTTAGCTGGACCA | |
| | | CGAACCAGGTGGCGTTGCGCAGGATGAAGGCGGAGATGAGGTTCCA | |
| | | GTGGATGATGTTTCGCAGGCACCGGATGCTCCTGGTGCAGCAGGGC | |
| | | GGGTGGATGATGGGAGCGATAGGAGAGAGAGGATGAGGGGTAGGC | |
| | | CACCTCCATCACCCCAGCCCAGATGTGTAGATGAGGAAACTGAGGC | |
| | | ACAGGGTGGGGCTGACTGGCCAAGTCACACAGGGAGGTGACAGCCA | |
| | | GGCTCTCCTAATGCCCCATGGAGCCCTTCCTGCCTGCAGCCCACCCA | |
| | | GACATAAGCCCCAGGCAGAGCCTGGCACTCCATGGAGCCTGTGCTC | |
| | | CATGGACCAGGGCA | |
| CRH | rs1912151 | AGTGGAATGTATGCCCTACGCCAGGCCCATGGAATCGGAGCTTGGTT | (SEQ ID NO: 24) |
| Receptor | | TTAGGAAAAAGCACCTCTGCAGTTCAGAAGCCCTGGTCCAACCACC | |
| (CRHR1) | | ACTCACCTCTCCCCACACGGTGA[A/G]CAGTGAACCTTGGTCCACAA | |
| | | ACCAGACCCCCAGAATCCGTTTCATGTCCCACCACGGTGTGCTCTCC | |
| | | CAGGCTGTGCCTGAGGCCCAGGCTCCTGTGCGCAGCAGAACGTGGG | |
| | | GAAAGGGGATAGAGTGTGTGCAAGTGTTGGGTGGGTGGGGGAAGGC | |
| | | TGGACGGTGGGGAAGGGAGTGAACAGTAGTAGCGGGAGGTGGTGG | |
| | | GGGGAGGAGAGGGTGCTGATACAGGCAGCAGGTGTAGGGGTGGTGC | |
| | | TGGGGGCTCAGTGCCATCCCCCGGCCAGCCATGCATGCAACAGTTGG | |
| | | GGGCCTG | |
| CRH | rs242924 | CTCTGCCTCATCGCACATGCATTTGTGGATTCGATGCCCAGACTAGC | (SEQ ID NO: 25) |
| Receptor | | CTGTGAGCCCCCTTGTTTCCCCCAGTGCCCTGCGTGGGGCCTGCTGT | |
| (CRHR1) | | GTAGTAGGTGCTTCATGAAAGAGTTGGTGTGAATCAAGGAGAGGCT | |
| | | TGAAGACTTAAATAGAAGGTCCACAAGCCTTCCAAAGACACTCAGG | |
| | | TGCAGGGACCCTCT[A/C]CATTTTTGCCCAGCAGCAGCCATGCCCAG | |
| | | GACCACACCCAAAGTTTTAAAAGACATGCCCTAGGCAGCCCAAACA | |
| | | CCTAGATTTGGGTCCCAGTCACTGCCATGTACTGGCTGCATGGTGCC | |
| | | ATGGGGGTTATGTAACTCTCCCATGCCTCAGTTTCCTCAACTGTACA | |
| | | AACGTTCGGCTTTACACTCTTGTGAGGATGAAACGAGATGATTATGC | |
| | | AAAAGCACTCTGTAAATGGTAAAGTGACACAGATATTAATAATATTT | |
| | | GCAGCTATGCTGTCTTTTCTATTGGAGAGATACAGAGCACGGCGCTG | |
| | | GAGACCACAACCAGAAGCTGGGCTGCCCGTGCTCCACTCAGGGCTC | |
| | | TGGCGCTTACTAGTTCTCTGTGCCTCAGTTTTCTTATGTGTAAAATGG | |
| | | GGGCAACAGCACCTACTTCAGAGGGACATTGTGCAAATTGGGTTAA | |
| | | TACAGCTCCAGTCCTTGATCAGTGCCTGGCACGGGTTAGCACCGTGG | |
| | | AGTATTACACTCTGGGCATTTTTAGCCACAGACTGTGGCAGCCTTTC | |
| | | CTCCGTCCCTTGATGCCTCCCCGAGTCACAACTCAGCA | |
| CRH | rs242925 | TGTCCCACAGTGCCTCTCCTGGTAAATTAAGGAATAGTGACTTTGCT | (SEQ ID NO: 26) |
| Receptor | | CTTTATTTAACAAGTACAATTGTTTCTCCATATCCATAGGTTTTGGGG | |
| (CRHR1) | | AAAAGTGTACTGAACACGTACAGACTTTTTTCTTGAAATTATTCCCT | |
| | | AAACAGTACAGTATAACAGCTATTTATATTTATATAACATTTACATT | |
| | | GTAGTAGGTATTAGAAATAATCTAGAGATGATTTAAAGTATACAGG | |
| | | AGGATGTGCATAGGTTATAT | |
| CRH | rs242936 | GGACACTTGTGAAATTGGCAAAATGGTTGCCTCTGGGAGGGAATCC | (SEQ ID NO: 27) |
| Receptor | | AGTGGCTGGGGACGGGGTGGGAGGGGACATTGTATTCCCATTTGTA | |
| (CRHR1) | | CCTTCTGAGTTCTGTGTAAGCAATATCTGCTCAAAAACATTTAACAT | |
| | | TTAAAAGGCTTTCCAAGGGCTTTTTCAAACTGCATTGCTACCTCCAT | |
| | | CCCGTCCCTGGTT[C/T]CCATACAGCCCTGTGGCTGGAACTGGATGTA | |
| | | TCTCTCAGGCATGTGTTGTGGAAAAGGGTGGGACTCCTGTGACCGAG | |
| | | TCTGGTCTCCCTTTGCACCCCCAGGACAGTCCCATGCCCAGTGCAAG | |
| | | CGGCTGCCCGATGAGCACAGGGAGAAGGAAGGAAGGAACAAGGAC | |
| | | CCATCTCATCACCATCAGAGCCTGCCCAGT | |
| CRH | rs242937 | AGAGGGGCTTCCTTACCACCCCCATCTTCCTGCCTCTTTCTGCCCATT | (SEQ ID NO: 28) |
| Receptor | | GGCCTCCAGCAGGGGTTGGTGGCTCCCCCGGCTCTGCCCACTCAAGC | |
| (CRHR1) | | TGGCATGATCATGCCCACAGGACTGGCCCGTGGACAGGCTTGACCA | |
| | | GCTCCCTGGGCTGTTCCTTTGCAAGTCATTATGTGGTGGCAGATGAC | |
| | | AGGTAGGTGCTCTTGTGAGAAA[C/T]CTCCTTTTCAGGGGTCTTGCAA | |
| | | GACAGCAAAAAAATGGTGAGACCTCATGAGTCAATTTTCACGAGGC | |
| | | TGGGCATGACACGGTGGAAGGGTATGCGGTGACAGCTCGCGTGGCA | |
| | | GGTGGCATGACAGGGCTTCTTGTGGTAGAGCCATCTCACAGAGGCTG | |
| | | CCGTGGGAGGTCCTCACTGGCTGTGCTGCACATGGGGGCTCAGAGTT | |
| | | CATGGGGCAGATCTTGGGGAAGGTCCCATGGTGGCACTTCCCCATAG | |
| | | CGGGCCTGTGTTGGAAGGTCGTGGTCTGTGGGTGGGGGGAACATCC | |
| | | ATGATGGAGGTCTCTTGGACAGAAAATGACAGGTGACAGGCGTGTG | |
| | | TGGGGAGATGTCATCACTCAGGTCTGTCACA | |
| CRH | rs242938 | CTCATCAGTAAATGGATTAATTAGTGCTTAATGATGAGGTGCAGCTC | (SEQ ID NO: 29) |
| Receptor | | TGGGTACCCACTTGTGACTGACTGCACGGGCGTGCTCCACCTCATCT | |
| (CRHR1) | | GGCGCTGGAGGCACCTGGAAAGGGGTCTTCCAGCCCTGCCCTCACA | |
| | | GACTGACCCCATGTCGAGAGGCCATCACCCCAGCTCCTTTCCTGGGA | |
| | | TCACAGAGGGAAG[C/T]GCGGGGGAGCCTAGAGAGCACCACACTCA | |
| | | ATCCTCCCACCCATTCTGCAGGTGTAGAAACTGAGGCCCACAGAAG | |
| | | GTCTGTGCCAAGGGCTACTTAACCAGGTGCAGATGCAGCTGGAATG | |
| | | AGAACTTAGGACTCCTGACTGCCACTTCAGGCAGGGCCAGCAAGTG | |
| | | AAGTGTGTGCCCAGGCACTGAGGGCTGAAAATGTTTATCTGGAGCAT | |
| | | GTCAAGTAACCCTTTGCAACGGGAGCAGAGGGAACTCATGGGGGGT | |
| | | GGCCAGGGCTGGCTGCAAAAGGTGACAGGGCCA | |
| CRH | rs242939 | CTCCTGACTGCCACTTCAGGCAGGGCCAGCAAGTGAAGTGTGTGCCC | (SEQ ID NO: 30) |
| Receptor | | AGGCACTGAGGGCTGAAAATGTTTATCTGGAGCATGTCAAGTAACC | |
| (CRHR1) | | CTTTGCAACGGGAGCAGAGGGAACTCATGGGGGGTGGCCAGGGCTG | |
| | | GCTGCAAAAGGTGACAGGGCCATGACCACAGACAGCCTGGCCGGCC | |
| | | CCACCCAGCAGCCTGAACACGGAGGCCACACAAGAGTGG[A/G]TTCC | |
| | | AAGTGAAGGAGTGACCAACTCAGATCTGAAACCTGAGGCTGGGCGG | |
| | | TGGCGCTGGGGAGTGGGGGCAGGTAGGCCCAGACACGAGGATCACT | |
| | | CTGGAAGTGGACATGACAGGAACTGGTGCCTCCTCCTGGAAGCTCCT | |
| | | CTGATGTGCAGATGCTGCCTCCTTCCTAGGGGCTCCAGAAGGACTCA | |
| | | GCCCCCAGCATCGTGAGAGTTATCGTCCCTGGGCTGTGACCCTGCCT | |
| | | CCAGGCTCTGCGACCTCCGATAGGTTATTAGCCTCTCTGTGCCTCAA | |
| | | TTTCCTTATTACAAAATGGGCCTGACTATATTGGGGGCTG | |
| CRH | rs242940 | AGAGAGAGAGCACGCGAGAGAGAGAGGATGTTGGGGCAGAGTTGA | (SEQ ID NO: 31) |
| Receptor | | GGCCCAGTGACACTTCAGGAGGGGAGGGTGGATATGGCCTCCAAAG | |
| (CRHR1) | | GGTGGGGGCTGGCACAGTCCTGGCACCCCCCTGAGGCTGCCCCTTCT | |
| | | TTCTCTGCCTTTTAGTGCCTCCCTCTGAGTGAGGCTGGCACACCAGTC | |
| | | CTTTTGAGCCCCAG[C/T]GTCCCCAGGTTAATAACCTAGAATTGGCAC | |
| | | AAGAGTGGACAGACAAGCCACGGAGGGCCAGGAACCATGAACCAG | |
| | | CGCGTGTGGGGGCAGCCTCTTCAGGCCTGGGCCGAGGCCTTAGCAG | |
| | | CTGCCAAGCCCTGGCTGGGGCTGCCTGCCATCTCCTCCCCAAATTAG | |
| | | CTTGTCCCCAGTCTCTCAGGAAACAGCACTGG | |
| CRH | rs242941 | GGCCTCCAAAGGGTGGGGGCTGGCACAGTCCTGGCACCCCCCTGAG | (SEQ ID NO: 32) |
| Receptor | | GCTGCCCCTTCTTTCTCTGCCTTTTAGTGCCTCCCTCTGAGTGAGGCT | |
| (CRHR1) | | GGCACACCAGTCCTTTTGAGCCCCAGTGTCCCCAGGTTAATAACCTA | |
| | | GAATTGGCACAAGAGTGGACAGACAAGCCACGGAGGGCCAGGAAC | |
| | | CATGAACCAGCGCG[G/T]GTGGGGGCAGCCTCTTCAGGCCTGGGCCG | |
| | | AGGCCTTAGCAGCTGCCAAGCCCTGGCTGGGGCTGCCTGCCATCTCC | |
| | | TCCCCAAATTAGCTTGTCCCCAGTCTCTCAGGAAACAGCACTGGGTT | |
| | | AAATTGGCTCCCTTTCTCTGCTGGACTCAGGGCAGTGCCGAGCAGCA | |
| | | CTTGTACCAAATGCTGGTTTTTCTTTCTAA | |
| CRH | rs242942 | AGGGCCATCCGAGGCAACGGCGTACCAGGCTGGGATTTTAACCTGT | (SEQ ID NO: 33) |
| Receptor | | CCTGGATCCTGCTGACAGTGTTGAGCCCGGGGACAGGGACTTGGGG | |
| (CRHR1) | | GGAGAGCTGGGGGCATGTGGCCGCGGGGAGGGGAGGATCGTATAG | |
| | | AGGCCATGCCCTGCTGTTTCAAAGCTCTTCATCTATTTCGTTTGATCT | |
| | | TCACACATCTGGCAATGTGATTATTTCCTTTCTACAGATGAAGAAAT | |
| | | TGAGGCCCAGGGAAGTTAGGTGACTTTTCCAAGGTCATGGGTCAATG | |
| | | GGTGAAAGAACCAAGACTCATAGGCAGGTATTTTGGACTCTAAATCT | |
| | | AGTGCTCTTTCCATTACGCCACATTGTGAGTCAGTCACAGGGGGTGA | |
| | | GGGACAGTTAAGGGGGCAGCAGGAAGGGAGCAGCGTTTGTTGAGGG | |
| | | CTGACTGTGTGCCAGGCACACATGACATATGCCATCTCATTT[A/G]AT | |
| | | GGCACCATTGTACCTTCCTCAGTAGCTTGCTCATTCACTCCTCACTCA | |
| | | TTCATCCACAGTATCTAGGGAGCTAGGCTCAAATGTAATGTTCCATG | |
| | | TGGCAGCGTCTGGAATGAACACACGGAACACACCCTCTCCATCAGT | |
| | | GTGGCCCCTGCCCTTCCTTCTTCCCTTCCCCGGCCAGTCTTCTTGCGC | |
| | | CTTGCTTTCGC | |
| CRH | rs242949 | TCACCTCCACAGGCAGGGTGGTCAGGGAGCCTGGCCGTCATCCCCCC | (SEQ ID NO: 34) |
| Receptor | | AGCCACAGCTCTTTGGGGGCTGCTCCATGACCTGCCAGCTCAGACTG | |
| (CRHR1) | | CTGTGGACTGCTTGATGCTGTGAAAGCTGACACGGGTTGGGGAGGT | |
| | | GGGGATGGACATGGCACGGGCCACTCGGGCACGGATCGAGTGCTTG | |
| | | TCCTGCCACCGGTGCCACCTCTTCCGGATGGCAGAACGGACCTGTGG | |
| | | GCACAGGGAGGAGCACGACATC | |
| CRH | rs242950 | GATGCGGACGATGTTGAAAAGGAAGATGAAATTGATCTGCAATTTG | (SEQ ID NO: 35) |
| Receptor | | AGCACACCAGCGGGCCGGAGTGTGCAAGAGGCTTGGGACCCAGGCT | |
| (CRHR1) | | TCTCAGCTCTGGCCTCAGTGCCCCTGCTGTTCTCCCTCCTCCTCATGA | |
| | | GCACCTGCACATCTCTGACCCATGCCCCCTCTTTGGGTGGCCCCCAC | |
| | | CTCTAGGTAGAGG[A/G]GTCCTTTCTGTCCACGGTTGGCACTGATTGC | |
| | | CCCTCCCCACTGGGCCCTGTCTCCTGCCCCTACCCAGGTTCTTACCAG | |
| | | CAGGACCAGGATCATGGGGCCCTGGTAGATGTAGTCGGTGTACACC | |
| | | CCAGGCCTTTTGCCAAACCAGCACCTAGAAGGCCACAGAGGAAAGG | |
| | | GGAGGAAGGGTCATCTGCGTCCACCTCGA | |
| CRH | rs242951 | CAGCAAGCTCCAGGGAGGCCTGGGGAGTGGGTGGGGACCAGACAA | (SEQ ID NO: 36) |
| Receptor | | ACTGGAAGCCCTTATGTCCTCGTGGGTATGAAGACCCCCAGTGGCTA | |
| (CRHR1) | | GATCTCATAAGCCAGAAATTGAGATTTGTAAATGAAACCTTTGGATT | |
| | | TTTTAAAGGTTGGCTACAAAATCGAGTTTTCAGAAAACACCATGCTG | |
| | | GCCACACACAGCTTCTGAGGGCCGGGTCCAGTCCTCGGGCCTCTGGT | |
| | | CTGCAAGTTCGTTTCTGGTTTCTCACACCCAGATCCTCTCTGTTGCAG | |
| | | TGTTGAATTCTCACCACCAGGTGGAGCTTCAGAGCCGCTCAGACTCA | |
| | | CTTTCTCCTTTAGCCCAAACAGTTTTTGTTTTGCTTTGTTTTTTGAGAC | |
| | | TCATGGAGTCTCACTCTGTTGCCCAGGCTGGAGTGCAGTGGCACGAT | |
| | | CTTGGCTCACTGCAACCTCTGCCTCCTGGGTTCAAGAGATTCTCCTGC | |
| | | CTCAGCCTTCCCGAGTAGCTGGACTACAGGTGCGCACCACCACACCC | |
| | | AGCTAATTTTTGTTTTTTTGTTTTTTTTTTAGTAGATACGGG[A/G]TTT | |
| | | CACCATGTTAGCCAGGCTGGTCTCAAACTCCTGACCTCAAGTGATCC | |
| | | GCCTGCCTCGGCTTCCCAAGGCCTCAGCCAGCCTGAGTCAGGAGGG | |
| | | AGTCAGGAGGGAGTCAAGCACATAAACACGCAAGATAATCTTCCAG | |
| | | GCTCCTCTTTCAGCCCAATGAAGCCGTGCAGGCCCCCTCCCCTCCAT | |
| | | GGGGGAGGAGAGGGCTTGTCACTGTGGTGGAGGCCACTTGGACGCC | |
| | | AGGTCCGTCAGTCAGATAGGTTCCCGGGAACACT | |
| CRH | rs242952 | CTGGGGTGCCTGGCCTTGAGGTGCCCT[C/T]GCAAGTCCCATTTCACA | (SEQ ID NO: 37) |
| Receptor | | GGCAGACTTCTGCGAATCCATTATCTGCGATACCCCAGGGCGCTGGA | |
| (CRHR1) | | GCTTAATTAGGGCTTAGCCCAGGCCCAGAGCTGAGAAACCTACCACT | |
| | | CAATTAATTGGATACTTTCCTCTGCAGTTCTGGGAGGGGTCTGGCGA | |
| | | GGCTGGGGGCTGGCAGAAGGGAATGGCATTTTCACTAATTAAACTA | |
| | | ATCGATTACCCAGAGTGCTAGGCACCAGGCCAGCAGGGGCTGCAGA | |
| | | GGAAAGAGATGGCAGAGCCAGGCACGGATGGGCTGGGGGGTGGGG | |
| | | CGGGTCACTCCCCCAGGTGTACACTACAACCTCCCCTGACCTCACAG | |
| | | GGGAGATGAGAGACAGGGGGCGGCAGGTGAATGGAACGTCTCCCAT | |
| | | AGGCCAAGACAGGCCAA | |
| CRH | rs717312 | CTGGGGTGCCTGGCCTTGAGGTGCCCTCGCAAGTCCCATTTCACAGG | (SEQ ID NO: 38) |
| Receptor | | CAGACTTCTGCGAATCCATTTATCTGCGATGCCGCCAGGCGCTGGAGC | |
| (CRHR1) | | TTAATTAGGGCTTAGCCCAGGCCCAGAGCTGAGAAACCTACCACTCA | |
| | | ATTAATTGGATACTTTCCTCTGCAGTTCTGGGAGGGGT[C/G]TGGCGA | |
| | | GGCTGGGGGCTGGCGGAGGGGAATGGCATTTTCACTAATTAAACTA | |
| | | ATCGATTACCCAGAGTGCTAGGCACCAGGCCAGCAGGGGCTGCAGA | |
| | | GGAAAGAGATGGCAGAGCCAGGCACGGATGGGCTGGGGGGTGGGG | |
| | | CGGGTCACTCCCCCAGGTGTACACTACAACCTCCCCTGACCTCACAG | |
| | | GGGAGATGAGAGACAGGGGGCGGCAGGTGAATGGAACGTCTCCCAT | |
| | | AGGCCAAGACAGGCCAACACCACCCTTCCATCCCCAGAAGGCAGAG | |
| | | ATCC | |
| CRH | rs739644 | CTGTGGGCCAGGAGCCCTAACCTACCTGAGGGGAGGCTGGGGGCCT | (SEQ ID NO: 39) |
| Receptor | | GATCCCTGCAATTGGGGCCTCCTGTGTGCACCCTTGGGTCCTTGTGG | |
| (CRHR1) | | TCTTGAACTCAGAGTCCCAAGAGGGCACAGGGGTGAGCCCAGACAC | |
| | | CATGTAGTTTACTCCAAGACTCACTGTGTGACCTCCCAGCACATTGT | |
| | | TGCCCCTCATCTGGCCCTCAGCCCCTCATCTGAGGATGGAGAGGGCT | |
| | | GGATGGCTTGGCTTCTAAGATGTCTTCCAGCTCAAAACTCCCAGATT | |
| | | CCTTCTCCTGCCCCTCTTTCCTCTACCAGATGGATTTGGGGGGTTAAG | |
| | | GTTGGGGGCTA[C/G]AGCAGAGGAGTAGGAAGACCCAGCCAGAAAG | |
| | | TGACTCCCCAGGGAGTGACTTGGGAGGCCAGGGCAGGGCAGGAGGC | |
| | | TGGGGCAGCCAGATCTAGCAGCCTCGTGTGTCTGTACCATGTCCTGG | |
| | | CCATGGGAGGGACTCGGGAGAGGGAGAAGACACACTGGGGAGGGG | |
| | | CTTGGGGGCCAAGGGGAGGAAGTGCAGAAAGGAAGAAGGCCTCTTG | |
| | | GCCAGGTCAGTCCAAGGGGTGCACAGTTTGGCCAGCCCCCAATATA | |
| | | GTCAGGCCCATTTTGTAATAAGGAAATTGAGGCACAGAGAGGCTAA | |
| | | TAACCTATCGGAGGTCGCAGAGCCTGGAGGCAGGGTCACAGCCCAG | |
| | | GACGATACTCTCACGATGCTGGGGGCTGAGTCCTTCTGGAGCCCCTA | |
| | | GAAGGAGCAGCATCTGCACATCAGAAGACTTTCAAGGAGGAGCACC | |
| | | GGTTCTGTCATGTTCACTTTCAGAGTGATCCTCGTG | |
| CRH | rs739645 | CTGTGGGCCAGGAGCCCTAACCTACCTGAGGGGAGGCTGGGGGCCT | (SEQ ID NO: 40) |
| Receptor | | GATCCCTGCAATTGGGGCCTCCTGTGTGCACCCTTGGGTCCTTGTGG | |
| (CRHR1) | | TCTTGAACTCAGAGTCCCAAGAGGGCACAGGGGTGAGCCCAGACAC | |
| | | CATGTAGTTTACTCCAAGACTCACTGTGTGACCTCCCAGCACATTGT | |
| | | TGCCCCTCATCTGGCCCTCAGCCCCTCATCTGAGGATGGAGAGGGCT | |
| | | GGATGGCTTGGCTTCTAAGATGTCTTCCAGCTCAAAACTCCCAGATT | |
| | | CCTTCTCCTGCCCCTCTTTCCTCTACCAGATGGATTTGGGG[G/T]GTTA | |
| | | AGGTTGGGGGCTACAGCAGAGGAGTAGGAAGACCCAGCCAGAAAG | |
| | | TGACTCCCCAGGGAGTGACTTGGGAGGCCAGGGCAGGGCAGGAGGC | |
| | | TGGGGCAGCCAGATCTAGCAGCCTCGTGTGTCTGTACCATGTCCTGG | |
| | | CCATGGGAGGGACTCGGGAGAGGGAGAAGACACACTGGGGAGGGG | |
| | | CTTGGGGGCCAAGGGGAGGAAGTGCAGAAAGGAAGAAGGCCTCTTG | |
| | | GCCAGGTCAGTCCAAGGGGTGCACAGTTTGGCCAGCCCCCAATATA | |
| | | GTCAGGCCCATTTTGTAATAAGGAAATTGAGGGACAGAGAGGCTAA | |
| | | TAACCTATCGGAGGTCGCAGAGCCTGGAGGCAGGGTCACAGCCCAG | |
| | | GACGATACTGTCAGGATGCTGGGGGCTGAGTCCTTCTGGAGCCCCTA | |
| | | GAAGGAGCAGCATCTGCACATCAGAAGACTTTCAAGGAGGAGCACC | |
| | | GGTTCTGTCATGTTCACTTTCAGAGTGATCCTCGTG | |
| CRH | rs81189 | CTGTGGGCCAGGAGCCCTAACCTACCTGAGGGGAGGCTGGGGGCCT | (SEQ ID NO: 41) |
| Receptor | | GATCCCTGCAATTGGGGCCTCCTGTGTGCACCCTTGGGTCCTTGTGG | |
| (CRHR1) | | TCTTGAACTCAGAGTCCCAAGAGGGCACAGGGGTGA[C/G]CCCAGAC | |
| | | ACCATGTAGTTTACTCCAAGACTCACTGTGTGACCTCCCAGCACATT | |
| | | GTTGCCCCTCATCTGGCCCTCAGCCCCTCATCTGAGGATGGAGAGGG | |
| | | CTGGATGGCTTGGCTTCTAAGATGTCTTCCAGCTCAAAACTCCCAGA | |
| | | TTCCTTCTCCTGCCCCTCTTTCCTCTACCAGATGGATTTGGGGGGTTA | |
| | | AGGTTGGGGGCTACAGCAGAGGAGTAGGAAGACCCAGCCAGAAAG | |
| | | TGACTCCCCAGGGAGTGACTTGGGAGGCCAGGGCAGGGCAGGAGGC | |
| | | TGGGGCAGCCAGATCTAGCAGCCTCGTGTGTCTGTACCATGTCCTGG | |
| | | CCATGGGAGGGACTCGGGAGAGGGAGAAGACACACTGGGGAGGGG | |
| | | CTTGGGGGCCAAGGGGAGGAAGTGCAGAAAGGAAGAAGGCCTCTTG | |
| | | GCCAGGTCAGTCCAAGGGGTGCACAGTTTGGCGAGCCCCCAATATA | |
| | | GTCAGGCCCATTTTGTAATAAGGAAATTGAGGCACAGAGAGGCTAA | |
| | | TAACCTATCGGAGGTCGCAGAGCCTGGAGGCAGGGTCACAGCCCAG | |
| | | GACGATACTCTCACGATGCTGGGGGCTGAGTCCTTCTGGAGCCCCTA | |
| | | GAAGGAGCAGCATCTGCACATCAGAAGACTTTCAAGGAGGAGCACC | |
| | | GTTCTGTCATGTTCACTTTCAGAGTGATCCTCGTG | |
| CRHBP | rs1053967 | CAGGGCCAGTTCACCTTCACCGCCGACCGGCCGCAGCTGCACTGCGC | (SEQ ID NO: 42) |
| | | AGC[C/T]TTCTTCATCAGCGAGCCCGAGGAGTTCATTACCATCCACTA | |
| | | CGACCAGGT | |
| CRHBP | rs13752 | GCCTTGAGCGCACGCGCGCACACACACACACACATACACACACGCA | (SEQ ID NO: 43) |
| | | TTAATTTTTGTACTTTGCTTCTTTTATGTTGTAATCTGTAAATGAAC | |
| | | ACATGG[A/C]AGAAAATAACCCCTGATTGGTAGGATCATAGTTCTAA | |
| | | ATGGAAATGTTTGTAATTTCTTTGATGTGCTACAAACCTGAAACTGGT | |
| | | AAGACAAGCACAAAGC | |
| CRHBP | rs1505079 | ATGGCTTGCATGAAGTACCACGCACCAGTCAAGGCACATGGTAGAC | (SEQ ID NO: 44) |
| | | ACTATAACTATGAGTGTTCCTTTCTCAGAAAACTTATCAGTCAAGTC | |
| | | TTTGGTA[A/G]TATAATTTTATCTTAAATGCTTCTAAAATGTTTTCTAT | |
| | | TTCAAGGAAATAGAGCTGGCTCCCTTAATTGATGAGAATTTATTTGG | |
| | | CAAAGAGAAAATAGC | |
| CRHBP | rs1700676 | GTAAGGCTGAAGGGAGTTGTAGGGAAAAGAAAGAGAGATCAGACA | (SEQ ID NO: 45) |
| | | GTTATTGTGCCTATGTAGAAAAGGAAGACATAAGAAACTCCATTTTG | |
| | | ACCTGTAC[C/T]CTGAACAATTGCTTTGCCCTGAGATGCTTGTTAATCT | |
| | | GTAACTTTGCCCCAACCTTGAGCTCACAAAAATATGTGTTGTATGGA | |
| | | ATCAAGGTTTAAGGGAT | |
| CRHBP | rs1715771 | TTCCAGCCTAGGCTGCGCTGGGTTCCTGCGCCCCGGGCGCGCCACCC | (SEQ ID NO: 46) |
| | | TCTCCCGCTCCTGGCGCGCCTCCGCGGACCGATCCTTAGCTAAGGGG | |
| | | ACCGCGGCCCTGGCGGTTCCGGCCAGCCCCTTCCCCGAGATGTCCGC | |
| | | GAGCCCTCTGCCCCCCGCACAGAGTCCCACCTTCCCCGCGTCCCGGG | |
| | | CGCCCGCGAGCC[C/G]GGCAGCCTCGACTCACGCAGAGCGCGGCGGT | |
| | | ACGGCTGCTCCCCAGCCAGCTCCCGCTTCAGGTTGGCGCTGAAGAGC | |
| | | AGGAAAGGATCGTAGTCCGCCGCTTCCCTCAGCTGGCAAGAAACAT | |
| | | AGGTCAGTCCGGAAAAGTTCAAGGGCTGGGGATAAAAAAGGGGACC | |
| | | AGGAGCGGGGCACCCTCCCTGCCACTCAGC | |
| CRHBP | rs1962640 | GAGGTTTCTGATTTCTAACTAAACAAAATATAGGGGAAACTCTTTAT | (SEQ ID NO: 47) |
| | | TTTGTCTTCTAGAATTCTTCTGTTGCAGTCCTCAGGAACAGCCTTTTT | |
| | | GGGGGGCTTTTTCAAAAATTCTTTTGTTTTTTCAGAACTAGAAATCA | |
| | | AAAGATCAGTAGTGCATTATTCTTTGGCAGATAAAGGCCCCATCTTT | |
| | | CAGCCGCATGAAGTGTTACCATTAATTCTACTGTATGCAAAAATGAG | |
| | | TGATATGTGTTCCTTTTCTAATTTTTTAAACAGATTTTTCTCTTAATGG | |
| | | GGAAAAAACAATTAGCAGATGAGTAAATTCATTTTTTTTTTTTTTTGA | |
| | | GACAAGAGTCTCCCTTTGTCACCCCGGCTGGAGCGCAGTGGCGCGAT | |
| | | CTCAGCTCACTGCAAGCTCCACCTCCCAGGTTCACGCCA[C/T]TCTCT | |
| | | CCCAAGTAGCTGGGACTACAGGCGCCCACCACCACGCCCAGCTAAT | |
| | | TTTTTGCATTTTTAGTAGAGATGGGGTTTCACTGCGTTAGCCAGGAC | |
| | | GGTCTGGATCTCCTGACCTCGTGATCCGCCTGCCTTGGCCTCCCAAA | |
| | | GTGCTGGGATTACAGGCGTGAGCCACCGCGGAGTAAATATTTTTAGT | |
| | | AAGATAATATGCTTTAGACCTGGGCTGTTCAGTTGTGTAGCATAA | |
| CRHBP | rs2135078 | TGGTGCAGAGCCAAATGCTGAACTAAATCATTTTGGACTTCTCAGAG | (SEQ ID NO: 48) |
| | | CTAGAACTCTGGGCCATTGAAGTAGAATTATTTAGAGGAGAGGTAG | |
| | | AATTGCA[A/G]TGTTTGCACAGGCAACACTAGCTGGTCCTGGCAGGG | |
| | | AAGCGGGTGCAGAGATTCATTTTCCGCTATCCTCAGAATACGTGTTT | |
| | | TTGTCACGATATACATT | |
| CRHBP | rs247742 | GTGGCAAACTCCATTGATGTCTTTATTTTAGGAAATTGCCACAGCTGT | (SEQ ID NO: 49) |
| | | TCCAGCTTTCAGTAACCACTGCCCTTATCAGTCAACAGCCCATCAAC | |
| | | ATCAAA[C/G]ACTCACCACCAGCAAAAAGATTATGACTCGCTGAAGG | |
| | | ATGGGATGACCTTTAGCATTTTTTAGCAATAAAGTATTTTTCTTTTCT | |
| | | TTCTTTTTCTTGTCT | |
| CRHBP | rs32895 | TAGGCACAATTTTAAATTCTGCACCTGCCCCCATGTCCATGGATTGA | (SEQ ID NO: 50) |
| | | ATATGGATCTGCTATTGTGTGGCCACCCTGGCCTTCAGGCTTAACAT | |
| | | AGGTGA[C/T]AATTTGCTCTGGGGCTTTGTGAAAGAAAAAATGTCTT | |
| | | ATTCCTACCTAACAAAAAGAAAGTATTAACCCTGCCTAACAATAGTC | |
| | | GAAGACCCAAAAAACA | |
| CRHBP | rs32898 | TTTTGTTTTTTCAGAACTAGAAATCAAAAGATCAGTAGTGCATTATT | (SEQ ID NO: 51) |
| | | CTTTGGCAGATAAAGGCCCCATCTTTCAGCCGCATGAAGTGTTACCA | |
| | | TTAATTCTACTGTATGCAAAAATGAGTGATATGTGTTCCTTTTCTAAT | |
| | | TTTTTAAACAGATTTTTCTCTTAATGGGGAAAAAACAATTAGCAGAT | |
| | | GAGTAAATTCATTTTTTTTTTTTTTTGAGACAAGAGTCTCCCTTTGTC | |
| | | ACCCCGGCTGGAGCGCAGTGGCGCGATCTCAGCTCACTGCAAGCTCC | |
| | | ACCTCCCAGGTTCACGCCATTCTCTCCCAAGTAGCTGGGACTACAGG | |
| | | CGCCCACCACCACGCCCAGCTAATTTTTTGCATTTTTAGTAGAGATG | |
| | | GGGTTTCACTG[C/T]GTTAGCCAGGACGGTCTGGATCTCCTGACCTCG | |
| | | TGATCCGCCTGCCTTGGCCTCCCAAAGTGCTGGGATTACAGGCGTGA | |
| | | GCCACCGCGGAGTAAATATTTTTAGTAAGATAATATGCTTTAGACCT | |
| | | GGGCTGTTCAGTTGTGTAGCATAAGCTACGTGTGTGTCCATCATAAA | |
| | | TGAATTGAATTAGAGTGGTGGGCTCAGAATCAGAAGGTAACATTGA | |
| | | GGTGCTATAATCCAACCCTCTGGTCAGGGCATGACATCGGTCTACCA | |
| | | CATTCAGGCACAGGCTGAAAGA | |
| CRHBP | rs905857 | CTGTTTTTCTGATTATTGGAATTTTCTTTTGACATGAAGGAAGTATCT | (SEQ ID NO: 52) |
| | | CATTGACAGAACTGCGTTGTGAAGGAGTGCTAACTGTAGCATAAAA | |
| | | TACAAAATTGGATTTTTAGATTGCAAAATACAGTAAAGCTTTGAAAA | |
| | | GTATTTGGCATGACATTTAACTCAATACATTTTGCCTAAAAAATATT | |
| | | AGCCAAGAACCCCTATCAACTTGTTTTTGAATAAACTTCTGTATGGA | |
| | | CCTTAAAATTCATGCTGAGTTTGACCG[C/G]ATTTTCTTGCACTGGTA | |
| | | GCATTTTCCCTCTGAGTCATCCTCATTTCCTTCTACTTTCTCACATGA | |
| | | CTAGGTTAAGATAACTCATGTATTTGCTGCTATCAAAGGCAGTCATA | |
| | | ATTCCTAATCAGACAGATTTTAGTGAAAACCAAATAAATAGAACTA | |
| | | GGACTGAGAAAAGGAGTATATCCAATTTCCTTTAAGCCCTAAATTCA | |
| | | TGGACTAGTGCCTGCTTTTTTTAAGTTGGAACTTAGTGGAAGAATAG | |
| | | TCCTTTGATAAGGACATTTTTGGGTATCTTTGGTACAGTTTTACT | |
| Eotaxin | G195a2_CL | CAACCCAGAAACCACCACCTCTCACGCCAAAGCTCACACCTTCAGCC | (SEQ ID NO: 53) |
| | | TCCAACATGAAGGTCTCCGCAGCACTTCTGTGGCTGCTGCTCATAGC | |
| | | AGCTGCCTTCAGCCCCCAGGGGCTC[G/A]CTGGGCCAGGTAAGCCCC | |
| | | CCAACTCCTTACAGGAAAGGTAAGGTAACCACCTCCAGAGCTACTA | |
| | | GGTCAGCAAGAATCTT | |
| Eotaxin | G195a3 | TAGTTTGACCTCTATGGTCCAATTCATTAATTTTCACAAGTGAGTGTT | (SEQ ID NO: 54) |
| | | CACTCCCAGCTCCCTGCCTGGGAGATTGCTGTAGTCATATCAATTTCT | |
| | | TCAA[G/A]TCAAGAGCAAAGATGGTTTTACTGGGCCTTTAAGAGCAG | |
| | | CAACTAACCCAAGAGTCTCATCCTTCCTCCTCTCCGTAGCAACCCTTT | |
| | | GTCCAGGGGCAGATGGTCCTTAAA | |
| Eotaxin | G195a4 | TTCATTAATTTTCACAAGTGAGTGTTCACTCCCAGCTCCCTGCCTGGG | (SEQ ID NO: 55) |
| | | AGATTGCTGTAGTCATATCAATTTCTTCAAGTCAAGAGCAAAGATGG | |
| | | [T/C]TTTACTGGGCCTTTAAGAGCAGCAACTAACCCAAGAGTCTCATC | |
| | | CTTCCTCCTCTCCGTAGCAACCCTTTGTCCAGGGGCAGATGGTCCTTA | |
| | | AATATTT | |
| FCER2 | G9782a0 | TTCATAGTGATGACAACACATTTAACATTTGTTTTGATTTCACCCTCT | (SEQ ID NO: 56) |
| | | CCTCTCTCCCCACTCTCAGTCTGCAGCCAGGAGAGCAGGGACGTCCT | |
| | | GTGCGAACTGTCAGACCACCACAACCACACTCTGGAGGAGGAATGC | |
| | | CAATGGGGACCCTGTCTGCAATGCCTGTGGGCTCTACTACAAGCTTC | |
| | | ACAATGTAAGTGGACTGGGATCAGCAAGAACAGGGCTCGCTTCCTG | |
| | | ATGGTGACCAGCAAACAG[C/T]GTCACCACCACCCTCTCCAAGTGAA | |
| | | TCGCTCACCATGGGGGCAGATGACAGGTTCCAAATAATTGATGCAAT | |
| | | AGGACCTAGCTTGGAAAACTACTTTGTCTAGCATAGCCGTGCTGAGG | |
| | | CCGAGGGGGCTCACAGCCTGGCAGCCACACAACCCCCTTGGTATGC | |
| | | ATTGGACACTCCACATACGATGCAGCAATCCGATGTGCTGAGTGGGC | |
| | | CTGTGTGGTTTATAAGGAAAAAAAAAAAATCTTCCTTTTGGAAAACA | |
| | | AAAAAAGCCACCGGTCCTATTTTGTTGTTTCCTTACATTTTAAACTCT | |
| | | TTGCAGAAAGAGAGAATGAAAGAGAAAGGTAAATAG | |
| FCER2 | G9782a10 | GTTGAATCCTTGGCTGAGGTCACACAGGCAAGGCAATTATAATAAA | (SEQ ID NO: 57) |
| | | CGCTGGTGATATTTTTAGCACAGATCACCAACCAACAGTATAGGTGT | |
| | | TCTTGGTATCATCATTTTACGAATGAGGAAAGAAGTTTCCAGGTTAA | |
| | | TTAAGCCATTTGTCAAGATCTCATGGGTGCACCTGCTGTGTAGCTCA | |
| | | AGTGTGATGGGCTCTGGAGTGCCCCACTTCTGATCTCAGGCTGCTAT | |
| | | CTCTCCACACCAGCTGTGTCTCCCTGCTAACCA[C/T]GCTAGTGAGTC | |
| | | CAGATTGTAGACTAAACAAAATCAGCAAATGGCCCCTGAGTGCNAC | |
| | | CAAGTCCCAGATGCTANCCTGTGCTGGTAACTAGGNTTTGATGGCTC | |
| | | ACCCTAACCATCATTAAATNCCAAATCAGCCAGAGCTGTGATTGTGC | |
| | | CCGCTGAGTGGACTGCGTTGTCAGGGAGTGAGTGCTCCATCATCGGG | |
| | | AGAATCCAAGCAGGACCGCCATGGAGGAAGGTCAATATTCAGGTAG | |
| | | GAGGACTCTCTGGTTCTAACGTTGGCAGAAGCAA | |
| FCER2 | G9782a12 | AATGAATCCTCCAAGCCAGGGTGAGTGCAGAGGGCCAGGGGCTTGA | (SEQ ID NO: 58) |
| | | GGTGGGACATCCAGATAGACCTTTGGGTGGGGTCTGGGAGAGGTTT | |
| | | GGAGGTCTGGATGTTGGATGACACCTGGGAAAGTGGCTGGGGAGTG | |
| | | GCTCCGATGTTGGGGAAGAACTGGGACTCAGTGTCCTGGGTTTAGGG | |
| | | AAGGGACTCCAGGTTGGGGACAGGTGCAGAGGTGTAAGGGTGGGCG | |
| | | TTGGGGCTCAGAGGGGAAGGAAAAAGCAGGACCTAGTCTTCTGGAA | |
| | | GTGAAGCTGGGGGCCTGGCATTGGTTGT[T/G]GGGGCTGAGGGAGTC | |
| | | TTAGCTCTTAGTCCCAGATCTGTCTCTGTGGNCAGTGACCCAGCNCT | |
| | | GAGTCAGGTAAGGAAGCTGTGCAAATGGAGCTGGGGNTCCACTGAG | |
| | | ACCCTTTGCTTCAGTGTGGGCTCTGGACAAGCTCCCAGGCTGTCGGG | |
| | | GGCTCTGGGTAATGGAGAGACGGACAGGGCCAGCATCCAGCTTCAC | |
| | | CCTGCACCCATAGTCAGTCCCTCCACCCCCGGCAGAGATCGAGGAGC | |
| | | TTCCCAGGAGGNGGTGTTGCAGGCGNGGGACTCAGATCGTGCTGCT | |
| | | GGGGCTGGTGACCGCCGCTCTGTGGGCTGGGCTGCTGACTCTGCTT | |
| FCER2 | G9782a13 | GGACATCCAGATAGACCTTTGGGTGGGGTCTGGGAGAGGTTTGGAG | (SEQ ID NO: 59) |
| | | GTCTGGATGTTGGATGACACCTGGGAAAGTGGCTGGGGAGTGGCTC | |
| | | CGATGTTGGGGAAGAACTGGGACTCAGTGTCCTGGGTTTAGGGAAG | |
| | | GGACTCCAGGTTTGGGGACAGGTGCAGAGGTGTAAGGGTGGGCGTTG | |
| | | GGGCTCAGAGGGGAAGGAAAAAGCAGGACCTAGTCTTCTGGAAGTG | |
| | | AAGCTGGGGGCCTGGCATTGGTTGTNGGGGCTGAGGGAGTCTTAGC | |
| | | TCTTAGTCCCAGATCTGTCTCTGTGGNCAGTGACCCAGC[C/T]CTGAG | |
| | | TCAGGTAAGGAAGCTGTGCAAATGGAGCTGGGGNTCCACTGAGACC | |
| | | CTTTGCTTCAGTGTGGGCTCTGGACAAGCTCCCAGGCTGTCGGGGGC | |
| | | TCTGGGTAATGGAGAGACGGACAGGGCCAGCATCCAGCTTCACCCT | |
| | | GCACCCATAGTCAGTCCCTCCACCCCCGGCAGAGATCGAGGAGCTTC | |
| | | CCAGGAGGNGGTGTTGCAGGCGNGGGACTCAGATCGTGCTGCTGGG | |
| | | GCTGGTGACCGCCGCTCTGTGGGCTGGGCTGCTGACTCTGCTTCTCC | |
| | | TGTGGCGTGAGGACACCCCCAGCTCCATGTGGTCCCCCCAACTCATG | |
| | | GGGCCCTTCTGTGTTCCCTCCTTCACCCCCATCTCAGAGCTGGGGGA | |
| | | GC | |
| FCER2 | G9782a15 | GGGGACAGGTGCAGAGGTGTAAGGGTGGGCGTTGGGGCTCAGAGGG | (SEQ ID NO: 60) |
| | | GAAGGAAAAAGCAGGACCTAGTCTTCTGGAAGTGAAGCTGGGGGCC | |
| | | TGGCATTGGTTGTNGGGGCTGAGGGAGTCTTAGCTCTTAGTCCCAGA | |
| | | TCTGTCTCTGTGGNCAGTGACCCAGCNCTGAGTCAGGTAAGGAAGCT | |
| | | GTGCAAATGGAGCTGGGGNTCCACTGAGACCCTTTGCTTCAGTGTGG | |
| | | GCTCTGGACAAGCTCCCAGGCTGTCGGGGGCTCTGGGTAATGGAGA | |
| | | GACGGACAGGGCCAGCATCCAGCTTCACCCTGCACCCATAGTCAGTC | |
| | | CCTCCACCCCCGGCAGAGATCGAGGAGCTTCCCAGGAGGNGGTGTT | |
| | | GCAGGCG[T/C]GGGACTCAGATCGTGCTGCTGGGGCTGGTGACCGCC | |
| | | GCTCTGTGGGCTGGGCTGCTGACTCTGCTTCTCCTGTGGCGTGAGGA | |
| | | CACCCCCAGCTCCATGTGGTCCCCCCAACTCATGGGGCCCTTCTGTG | |
| | | TTCCCTCCTTCACCCCCATCTCAGAGCTGGGGGAGCCCAACGCATCC | |
| | | TCTCAAAACCCAATTTTCCCATCCTGCCTCCCATCTTGCCACTGCCAG | |
| | | CCCTTTCCCATCCCCCACCCTCCAGAGCCCCTCACCCCACACCCAGC | |
| | | ATCCCTGTCCTCCACACTTTCAGCACCTCCATGGCTTATACCCA | |
| FCER2 | G9782a17 | ACAGAGTCTAAAACAGCTGGAAGAGAGGGCTGCCNGGAACGGTATG | (SEQ ID NO: 61) |
| | | AAGGGGTCAAGGTGGAGGGGGTGGGGGTGAGGGTTAGGATGCCCA | |
| | | GCACTCATCCCGCTCCTCTCTGGGCCTCAGTGTCCCCTCGCTAAGGA | |
| | | ACTGAAGGTCAAACTAAGTTGCTCCCTGTCCTAGGGAAGGCGGGGA | |
| | | CCCAGGGAAGCTTGGAAAAGTGCCTGATAGCATCCTAATACAGATG | |
| | | CTCTTCC[G/T]CTTGCAATGGGGTTATGTCCCCATAAGCGCAAAGTAC | |
| | | GTTGAAAATAGTTTAAGTCAAAACTGTGTGGCTGGCTGAAGGCTTTG | |
| | | GCTCCCTACCACTGCCCAGCATTGCAAGAGAGTTAAGGTTTCCACTA | |
| | | AACGCATATTGGTTTCAGACCATTGTAAAGTCGAGCCATTGTAACTC | |
| | | GAGCTATTGTAAGTTGAGCCATTGTAAGTTGGCAGCCTTAAGCATCT | |
| | | GTAGTGTAAGGATTTTGTAACTGCTTTGTGCCT | |
| FCER2 | G9782a19 | GGGAAGGAGGGAGTGTCTCAGAGCCCTGGGGAACCACGAGGCTGGG | (SEQ ID NO: 62) |
| | | GGGCCAGGCTTGGGGGGCACCCTCGGCCTGTACACATCCTCCCTGAG | |
| | | ACCAGCCCTGCGCTCCTGCACACACCAGACTTGGAGCTGTCCTGGAA | |
| | | CCTGAACGGGCTTCAAGCAGATCTGAGCAGCTTCAAGTCCCAGGGT | |
| | | GAGGCTTGGGAGGGGATGGGGCAGTGGGGGAGGGAACGGACAAGG | |
| | | AGGGGGCCTGCCAGTTGGTCTCTGAGCACCGCCCCTT[T/C]GACTC | |
| | | CCCAAGAATTGAACGAGAGGAACGAAGCTTCAGATTTGCTGGAAAG | |
| | | ACTCCGGGAGGAGGTGACAAAGCTAAGGATGGAGTTGCAGGTGTCC | |
| | | AGCGGTGAGTGTGTGAGTCTCTGCTCAGGACGACGCGTGACCTCACC | |
| | | NGGGGCTCGGGCGTGTTGGCAAATGTGACGGCACGCACGTGTGACG | |
| | | TGAGGCAGCATGTCTATACGTTTGTGTGACCCGAGTGTCATCGGGAG | |
| | | GACACAAAGATATAAGCGCAGGGTGGGGAGTGTGCATGGATGCGAG | |
| | | GCAGAGGTGTGCGTGATGACGGAAATAGGACTAAGTTTCTGA | |
| FCER2 | G9782a20 | GGGAAGGAGGGAGTGTCTCAGAGCCCTGGGGAACCACGAGGCTGGG | (SEQ ID NO: 63) |
| | | GGGCCAGGCTTGGGGGGCACCCTCGGCCTGTACACATCCTCCCTGAG | |
| | | ACCAGCCCTGCGCTCCTGCACACACCAGACTTGGAGCTGTCCTGGAA | |
| | | CCTGAACGGGCTTCAAGCAGATCTGAGCAGCTTCAAGTCCCAGGGT | |
| | | GAGGCTTGGGAGGGGATGGGGCAGTGGGGGAGGGAACGGACAAGG | |
| | | AGGGGGCCTGCCAGTTGGTCTCTGAGCACCGCCCCTTGT[T/C]GACTC | |
| | | CCCAAGAATTGAACGAGAGGAACGAAGCTTCAGATTTGCTGGAAAG | |
| | | ACTCCGGGAGGAGGTGACAAAGCTAAGGATGGAGTTGCAGGTGTCC | |
| | | AGCGGTGAGTGTGTGAGTCTCTGCTCAGGACGACGCGTGACCTCACC | |
| | | NGGGGCTCGGGCGTGTTGGCAAATGTGACGGCACGCACGTGTGACG | |
| | | TGAGGCAGCATGTCTATACGTTTGTGTGACCCGAGTGTCATCGGGAG | |
| | | GACACAAAGATATAAGCGCAGGGTGGGGAGTGTGCTTGGATGCGAG | |
| | | GCAGAGGTGTGCGTGATGACGGAAATAGGACTAAGTTTCTGA | |
| FCER2 | G9782a22 | AGCATCATAGCTCCAGCAGAGAACACAGCCCGTGAGGCTGTCTGTT | (SEQ ID NO: 64) |
| | | AGGCCCTGGGGTGGGTCTGCTTTTAGCCGGGACCCCAGGAGTGGCCC | |
| | | TAGGAGGGGTGCTGCCACCTAGTCTGCCCAGGGGTGCCCAAGGCAC | |
| | | TTCCATTGGCCCCACCCCCGAGCCTCTCCTCCACCCCAGGCTTTGTGT | |
| | | GCAACACGTGCCCTGAAAAGTGGATCAATTTCCAACGGAAGTGCTA | |
| | | CTACTTCGGCAAGGGCACCAAGCAGTGGGTCCACGCCCGGTATGCCT | |
| | | GTGACGACATGGAAGGGCAGCTGGTCAGCATCCACAGCCCGGAGGA | |
| | | GCAGGTGGGC[T/C]TGGGGCTCTGCAGAGGTGGTGGGCAGCATGGCGA | |
| | | GGGTGGGGGGACCCCCACCCCACTCTACCCAACCTCTCGAAGTGGG | |
| | | CTGGAAGGCCCCGGGCACATGTCTCCAGTCCTCCAGCCCTGTCCTGN | |
| | | CCCCCAGGACTTCCTGACCAAGCATGCCAGCCACACCGGCTCCTGGA | |
| | | TTGGCCTTCGGAACTTGGACCTGAAGGGGGAGTTTATCTGGGTGGAT | |
| | | GGGAGCCACGTGGACTACAGGTGAGGAGGGGGCCTCTGGGATCCAG | |
| | | GGGAGGAGATGGAAATACCGTGGAGGGAGGAGCTCCCTAGATA | |
| FCER2 | G9782a26 | CTGGTCAGCATCCACAGCCCGGAGGAGCAGGTGGGCNGGGGCTCTG | (SEQ ID NO: 65) |
| | | CAGAGGTGGTGGGCAGCATGGCGAGGGTGGGGGGACCCCCACCCCA | |
| | | CTCTACCCAACCTCTCGAAGTGGGCTGGAAGGCCCCGGGCACATGTC | |
| | | TCCAGTCCTCCAGCCCTGTCCTGNCCCCCAGGACTTCCTGACCAAGC | |
| | | ATGCCAGCCACACCGGCTCCTGGATTGGCCTTCGGAACTTGGACCTG | |
| | | AAGGGGGAGTTTATCTGGGTGGATGGGAGCCA[C/T]GTGGACTACAG | |
| | | GTGAGGAGGGGGCCTCTGGGATCCAGGGGAGGAGATGGAAATACCG | |
| | | TGGAGGGAGGAGCTCCCTAGATAAACTGCATCGGAAAGCGGATGAG | |
| | | GGCTCAGAGATAAGGGTCTCTAGGGTGCAGGGGAGAAGGACGCCAG | |
| | | TGGGGGCTGGGGGACCTGGCCAAGGCTTCTCTGACCTGGAGGAGGG | |
| | | GATATAGAGGAAGGGGCTCAGGGAGGAAGTTCATG | |
| FCER2 | G9782a5 | ATCATCATTTTACGAATGAGGAAAGAAGTTTCCAGGTTAATTAAGCC | (SEQ ID NO: 66) |
| | | ATTTGTCAAGATCTCATGGGTGCACCTGCTGTGTAGCTCAAGTGTGA | |
| | | TGGGCTCTGGAGTGCCCCACTTCTGATCTCAGGCTGCTATCTCTCCAC | |
| | | ACCAGCTGTGTCTCCCTGCTAACCACGCTAGTGAGTCCAGATTGTAG | |
| | | ACTAAACAAAATCAGCAAATGGCCCCTGAGTGCNACCAAGTCCCAG | |
| | | ATGCTANCCTGTGCTGGTAACTAGGNTTTGATGGCTCACCCTAACCA | |
| | | TCATTAAAT[C/T]CCAAATCAGCCAGAGCTGTGATTGTGCCCGCTGAG | |
| | | TGGACTGCGTTGTCAGGGAGTGAGTGCTCCATCATCGGGAGAATCCA | |
| | | AGCAGGACCGCCATGGAGGAAGGTCAATATTCAGGTAGGAGGACTC | |
| | | TCTGGTTCTAACGTTGGCAGAAGCAATGACCCTTAGCTACTGCCTTT | |
| | | CACCCAGAAGAGAAGCGGGGCTCCCCAGTCCCTCTCTGGGAAAGAG | |
| | | GGTGAATTTCTAAGAAAGGGACTGGTGTGAGTA | |
| FCER2 | G9782a8 | GTGCTCCATCATCGGGAGAATCCAAGCAGGACCGCCATGGAGGAAG | (SEQ ID NO: 67) |
| | | GTCAATATTCAGGTAGGAGGACTCTCTGGTTCTAACGTTGGCAGAAG | |
| | | CAATGACCCTTAGCTACTGCCTTTCACCCAGAAGAGAAGCGGGGCTC | |
| | | CCCAGTCCCTCTCTGGGAAAGAGGGTGAATTTCTAAGAAAGGGACT | |
| | | GGTGTGAGTAAGGAGGTGAGGCCGGACTGACTTTCCTGGCACAGAG | |
| | | CCAGGAAGGAGTGG[C/A]AAATTGAGGGCCCCTCCTTTTTCTGATTC | |
| | | AACACCCTCCTGACAAAAAAAGAAAAAGAAAAAAAAAAAAAAACG | |
| | | GCTTCAGCTAGGGAGCGGGGAGCCCAATAGAGTCAGAGGCCAAATA | |
| | | GAACAGGAACTTTGGAACAAGCAGAATTTAGCATAATGAATCCTCCA | |
| | | AGCCAGGGTGAGTGCAGAGGGCCAGGGGCTTGA | |
| FCER2 | rs1042428 | CAGGTGTCCAGCGGCTTTGTGTGCAACACGTGCCCTGAAAAGTGGAT | (SEQ ID NO: 68) |
| | | CAA[C/T]TTCCAACGGAAGTGCTACTACTTCGGCAAGGGCACCAAGC | |
| | | AGTGGGTCCA | |
| FCER2 | rs1042429 | GCCAGCCACACCGGCTCCTGGATTGGCCTTCGGAACTTGGACCTGAA | (SEQ ID NO: 69) |
| | | GGG[A/G]GAGTTTATCTGGGTGGATGGGAGCCATGTGGACTACAGCA | |
| | | ACTGGGCTCC | |
| FCER2 | rs1990975 | TTAAAGCATCTACTATGTGCTGAGGAGCATGGGACTTGGGGTCCTTC | (SEQ ID NO: 70) |
| | | AGGCCTGGAGGTCACGGGCTACCTTCTTTCGCTGCTTGACTATAGGT | |
| | | AAGTCCCTTCTCTCGGAGCCTCAGGGAGGTGGGGTGTGGCGCGGCC | |
| | | AGGCTCTGATTAGCGGCAGCTCAAGCTGGCCCTTTTGACATTGTGAG | |
| | | AGGAACCCAGAGACCATCCTGGGACTGGAACTGTCCAGGGAGGCAG | |
| | | CTGGACAGACCTGAGTCCGAGTCCTGGCTTCT[C/T]CCTGGATGAGCA | |
| | | GCTCAGTTTGCTCATCTGTAAATTGGGAGTATTCAAAGCACCAGCAC | |
| | | ACAGTAGGTGCTCAGTAAACCTACTGGCAGGATGCTGGGGGACTGT | |
| | | CCTCATTCTGTGGGGCCTCTCTCAGTGGAACTGAGGTTCCAAGAGGG | |
| | | TAAGTCATTTGTCCCCATCAC | |
| FCER2 | rs2229230 | CTGGTCTTGAATCAGGTCCCATGGACTCCGCGGAACCTTCGCTGGCT | (SEQ ID NO: 71) |
| | | GGCGGCGTGCATGTGGCCAGCCGGTCGCACACCCAGGCGCCCAGCT | |
| | | TACGGTC[A/G]CAGAAGGCGTCGTTCCAGCGACCGGAGCCCCGCATC | |
| | | ATCACGCAGTCCTCGCCCTGGCTCCGGCTGGTGGGCTCCCCTGGAGC | |
| | | CCAGTTGCTGGAGCAAA | |
| FCER2 | rs2335523 | CCTGGCAACAGAGCAAGATCTCCACCTCAAAAAAAAAAAAAATTGT | (SEQ ID NO: 72) |
| | | TTTCTTGTAGAGACGAGGGTCTCGCTATGTTGCCCAGGCTGGTCCCC | |
| | | AGCTACC[A/G]GCCTTAAGCAATCCTCCTGCCTCGGCTTCCCAAAGCG | |
| | | TTGGGATTACAGGCATGAGCCCACATGTCCTGCTCTTCCTTCATCTTC | |
| | | ACAGCCAGAAGCACA | |
| FCER2 | rs6952 | GCGGCCGCGCACCGAGGTGAGCCTGAGCGCCTTCGCACTGCTGTTCT | (SEQ ID NO: 73) |
| | | CCGAGCTGGTACAGCACTGCCAGAGCCGCGTCTTCTCCGTGGCCGAG | |
| | | CTGCAG[G/T]CGCGCCTGGCCGCGCTGGGCCGCCAGGTGGGCGCGCG | |
| | | CGTGCTGGATGCGCTGGTGGCGCGCGAAAAGGGTGCCCGGCGTGAG | |
| | | ACCAAGGTGCTAGGCGC | |
| FCER2 | rs753733 | CAGGGGGGTGTCTTCTGGAGTGAGAGACATGGGAGGTAGCCCACCC | (SEQ ID NO: 74) |
| | | AGGAGTGGGGATTTGGAGTTCCCCAGTTGAGAGTGGGGAGGGTGTT | |
| | | AAGGATCA[A/G]GGGACACATTTTGGGAAGGATGAGGAGGGTGGGC | |
| | | AAAGTGCCAAGGGGTCTTAGTATATAGAAGTCTAGGTGGCATAGTG | |
| | | GCTAGGGCTGCAGATGCCTG | |
| FCER2 | rs889182 | GGGCTGATGACAGCACCCAACTCATGAGGCCAAGATAGGATCCAAT | (SEQ ID NO: 75) |
| | | GAGATCACAGCATACCTGAGGTTTTTGGTGCCGGGCTGCGCACAGTG | |
| | | GGTCTTC[A/G]GATTCTAAACATTTATGAAACATCTACTCTGTGCCAC | |
| | | GTCCCACTGAGAAATGCTAAGAGCCCATAACCCAGGCCTCAGCCTTC | |
| | | TCCTCTGCCAATGTCC | |
| IL18BP | G9772a3 | GAGCCTGCTGGCCTCTGGCCATCTCAGAGGAGCAGCAGCCATCCAG | (SEQ ID NO: 76) |
| | | CACTGCCTTTGTCACCTGGGCTCCCAAGTCACCGAGGCTGGGCACTA | |
| | | GAAAAGGTCATCCTGAGGAGACAGGTTCAGAAGAGGATTCATCACG | |
| | | TGAACCAAGGACCATTCCTCACATTCCCCGTGTTTAGGGCTAGGGCC | |
| | | TCTCGGAGACAACTGCACTTCTGTAACGGACGTTCCCACCTAGGTGG | |
| | | TGTGCAGAGCAGTTCTCTAGGTTCCAGATGCATGGGGACTGGGGGG | |
| | | AGCTGGCAG[G/A]GAGGGCACAGCAGAGCAGGGTAGGGGAAGGGCC | |
| | | TGCTCTTCTGAAGAGCTAACTGCTGCCTGTGTCCCTAGATGGAACGC | |
| | | TGAGCTTATCCTGTGTGGCCTGCAGCCGCTTCCCCAACTTCAGCATC | |
| | | CTCTACTGGCTGGGCAATGGTTCCTTCATTGAGCACCTCCCAGGCCG | |
| | | ACTGTGGGAGGGGAGCACCAGGTGAGGGTCGCAGCAGCCAGGTGGG | |
| | | TGGGAAGGAGGCCTTCTGCGGCCTTCTCATGACCTTT | |
| IL18BP | G9772a6 | CCCCCCCAGCTCTGTTTCTAAGTGCTGAAAGAGCTCCAGGCTATGCT | (SEQ ID NO: 77) |
| | | ACGGGAGGAGAAGCCAGCTACTGAGGAAAAGCCAGCTACTGAGAA | |
| | | AAAGCGGGAGTGGTTTACCATTCTCCTCCCCCACCTTTCACCAGAGA | |
| | | AGAGGACGTTGTCACAGATAAAGAGCCAGGCTCACCAGCTCCTGAC | |
| | | GCATGCATCATGACCATGAGACACAACTGGACACCAGGTAGGCCTT | |
| | | GGGGCTAC[G/C]CATGGGCAGGCGGGGTAGGGTGAGGTCTATGAAC | |
| | | AGAATGGAGCAATGGGCTAACCCGGAGCCTTCACTCCAAGGCAAAC | |
| | | CACCCAGCGCACCTGGTGCTGTTGCTTTAAGAACCTGGGCAGATATT | |
| | | GTAGCTCTGGCTCCAGTCTAAAGCTTCTCTGTACTCTGTTCAATAAA | |
| | | GGGCTAAGGGGTGGGTGCTGAGGGGTCCCTCTTCCCGCTCTGATTCC | |
| | | CTGGCTAGAACCCAGACATCTCTGGGCTGGAGTTACATCCTTACCCG | |
| | | GGCAGCCCACTCTGTCTCCAGAGCCGCTGACCTGTAAC | |
| IL18BP | rs1053725 | TGGGCCTGGCCAGTCTTCCTCTTAGCCTCTGGATCTAGAAGGGACCA | (SEQ ID NO: 78) |
| | | TAA[A/G]AGGAGTAGGCCCTGGTTCCTGCTGTCCTGGTGGCTGGGCC | |
| | | CAGCAGGGGC | |
| IL18BP | rs1062452 | CTGTCTACCTGGAGTGAACAGTCCCTGACTGCCTGTAGGCTGCGTGG | (SEQ ID NO: 79) |
| | | ATG[A/C]GCAACACACCCCCTCCTTCTCTGCTTTGGGTCCCTCTCTC | |
| | | ACCAAATTC | |
| IL18BP | rs14537 | CAGCCTTCAGCAGCAGCTCACACCCTACCTTCCCCAGACTTGCACTG | (SEQ ID NO: 80) |
| | | GGGTGGGATTTGGAGTGATGGGAAGGTTTTTAAGGGCCGGGGATGG | |
| | | ATCTTTT[C/T]TAAATGTTATTACTTGTAAATAAAGTCTATTTTT | |
| IL18BP | rs1541304 | AGACAGGGGCGTGGGAGCAGGAAGAGATCTTCCCAGCCAGTGGGTG | (SEQ ID NO: 81) |
| | | CAGGCTAGTATCGCAAGTTCTCATCTGGCCATGTGACTGTGTCTCTTT | |
| | | CAGGCAAAGCACTAAAGGGCCAGTACCAGTGAGTGGCCCCACCTGT | |
| | | GTCCCCGATGCTGACCTCACCTGGTCCTCCGCCTACTGTCCCTCTCAG | |
| | | TGCCTTCTCTCAGCTCCCAGGCCAACAGTAGCCAAACCCCTAGAGAC | |
| | | AGTGATGCCTGCCCGCACCCTGGCCTGGTCCCTGGTCCTTCACTGGC | |
| | | GCCTTCTCGGAGCTGGCCCAGGGGGCCTGGAGCATGGACAGTGTGG | |
| | | GCGCTCTCCCTACCTTGCCTCCTTTTTTCTTAAAGCAAAGTCACTTCT | |
| | | CCATCACAACCAGATTTGAGGCTGGTTTTGATGGCTGGGTCCTTGGG | |
| | | CCTGGCCAGTCTTCCTCTTAGCCTCTGGATCTAGAAGGGACCATAAG | |
| | | AGGAGTAGGCCCTGGTTCCTGCTGTCCTGGTGGCTGGGCCCAGCAGG | |
| | | GGCCCTCACTCTTGAAGTCCAGGACTGGGTCTGACCTGGTGGGAGCA | |
| | | CCTGCCAGAGGATGCTCTTTCCCAGGACGGATGGGCCCT[A/G]TGTCT | |
| | | CAGGAGTGGGGTTGGGGGACAGCCTTCAGCAGCAGCTCACACCCTA | |
| | | CCTTCCCCAGACTTGCACTGGGGTGGGATTTGGAGTGATGGGAAGGT | |
| | | TTTTAAGGGCCGGGGATGGATCTTTTCTAAATGTTATTACTTGTAAAT | |
| | | AAAGTCTATTTTTCTCCCGTGAGTACTGAGTTGACTGATCGGGTGTG | |
| | | GGAAAAAGAGGA | |
| IL18BP | rs1573503 | TGCTCTGCACACCACCTAGGTGGGAACGTCCGTTACAGAAGTGCAGT | (SEQ ID NO: 82) |
| | | TGTCTCCGAGAGGCCCTAGCCCTAAACACGGGGAATGTGAGGAATG | |
| | | GTCCTTGGTTCACGTGATGAATCCTCTTCTGAACCTGTCTCCTCAGGA | |
| | | TGACCTTTTCTAGTGCCCAGCCTCGGTGACTTGGGAGCCCAGGTGAC | |
| | | A[A/G]AGGCAGTGCTGGATGGCTGCTGCTCCTCTGAGATGGCCAGAG | |
| | | GCCAGCAGGCTCTGGGTGG | |
| IL18BP | rs1892919 | CGTACCTGTGCTCCCACGTTCCCGGCTGGAGCGGAAGGGAAGGAAA | (SEQ ID NO: 83) |
| | | GGTCATGAGAAGGCCGCAGAAGGCCTCCTTCCCACCCACCTGGCTGC | |
| | | TGCGACCGTCACCTGGTGCTCCCCTCCCACAGTCGGCCTGGGAGGTG | |
| | | CTCAATGAAGGAACCATTGCCCAGCCAGTAGAGGATGCTGAAGTTG | |
| | | GGGAAGCGGCTGCAGGCCACACAGGATAAGCTCAGCGTTCCATCTA | |
| | | GGGACACAGGCAGCAGTTAGCTCTTCAGAAGAGCAGGCCCTTCCCC | |
| | | TACCCTGCTCTGCTGTGCCCTC[C/T]CTGCCAGCTCCCCCCAGTCCCC | |
| | | ATGCATCTGGAACCTAGAGAACTGCTCTGCACACCACCTAGGTGGG | |
| | | AACGTCCGTTACAGAAGTGCAGTTGTCTCCGAGAGGCCCTAGCCCTA | |
| | | AACACGGGGAATGTGAGGAATGGTCCTTGGTTCACGTGATGAATCCT | |
| | | CTTCTGAACCTGTCTCCTCAGGATGACCTTTTCTAGTGCCCAGCCTCG | |
| | | GTGACTTGGGAGCCCAGGTGACAAAGGCAGTGCT | |
| IL18BP | rs2032353 | AGACCCCTCCTGCTCCTTCACCCTCGGTCTTCCTGCTACAGACATCTC | (SEQ ID NO: 84) |
| | | TATGCTGTGCTCGGGGTTAGAGCCCCAGAAAGTGATGGGCAAGTGT | |
| | | CCTAAG[A/C]CCACAATCCTCCCTAACCTGTGGGGGCTGCCTCGCATA | |
| | | TCACCTTCAAGGCTTCCATAAAGCTGTACTCTGGGAAAAAGGGACAC | |
| | | CATGTTAAACAAGGC | |
| IL18BP | rs2155145 | GACTCAGCAGGAGCGATAGCTTCCACGAAGCCTTTCTTGACCACTTG | (SEQ ID NO: 85) |
| | | CTTTTTATTCCAACTGTGCCCTAAATAGATATCTGGTATAATACCCGT | |
| | | TTTTC[G/T]TTATTCTTCTCTAAGAATAAATTTAGATCACATCTTATTT | |
| | | ATCTCACTAGGATACAGCCTGATAAACAGCAGGCACTCCATAAACA | |
| | | GACATAGGGAGGAA | |
| IL18BP | rs760246 | GCCACAGCAGTCCACAGCAGCAGGGTTAAGACTCAGCACAGGGCCA | (SEQ ID NO: 86) |
| | | GCAGCAGCACAACCTTGACCAGAGCTTGGGTCCTACCTGTCTACCTG | |
| | | GAGTGAACAGTCCCTGACTGCCTGTAGGCTGCGTGGATGCGCAACA | |
| | | CACCCCCTCCTTCTCTGCTTTGGGTCCCTTCTCTCACCAAATTCAAAC | |
| | | TCCATTCCCACCTACCTAGAAAATCACAGCCTCCTTATAATGCCTCCT | |
| | | CCTCCTGCCATTCTCTCTCCACCTATCCATTAGCCTTCCTAACGTCCT | |
| | | ACTCCTCACACTGCTCTACTGCTCAGAAACCACCAAGACTGTTGATG | |
| | | CCTTAGCCTTGCACTCCAGGGCCCTACCTGCATTTCCCACATGACTTT | |
| | | CTGGAAGCCTCCCAACTATTCTTGCTTTTCCCAGACAGCTCCCACTCC | |
| | | CATGTCTCTGCTCATTTAGTCCCGTCTTCCTCACCGCCCCAGCAGGGG | |
| | | AACGCTCAAGCCTGGT[C/T]GAAATGCTGCCTCTTCAGTGAAGTCATC | |
| | | CTCTTTCAGCTCTGGCCGCATTCTGCAGACTTCCTATCTTCGTGCTGT | |
| | | ATGTTTTTTTTTTCCCCCTTCACTCTAATGGACTGTTCCAGGGAAGGG | |
| | | ATGGGGGCAGCA | |
| IL18BP | rs949323 | CAGCCAGTGGGTGCAGGCTAGTATCGCAAGTTCTCATCTGGCCATGT | (SEQ ID NO: 87) |
| | | GACTGTGTCTCTTTCAGGCAAAGCACTAAAGGGCCAGTACCAGTGA | |
| | | GTGGCCCCACCTGTGTCCCCGATGCTG[A/C]CCTCACCTGGTCCTCCG | |
| | | CCTACTGTCCCTCTCAGTGCCTTCTCTCAGCTCCCAGGCCAACAGTA | |
| | | GCCAAACCCCTAGAGACAGTGATGCCTGCCCGCACCCTGGCCTGGTC | |
| | | CCTGGTCCTTCACTGGCGCCTTCTCGGAGCTGGCCCAGGGGGCCTGG | |
| | | AGCATGGACAGTGTGGGCGCTCTCCCTACCTTGCCTCCTTTTTTCTTA | |
| | | AAGCAAAGTCACTTCTCCATCACAACCAGATTTGAGGCTGGTTTTGA | |
| | | TGGCTGGGTCCTTGGGCCTGGCCAGTCTTCCTCTTAGCCTCTGGATCT | |
| | | AGAAGGGACCATAAGAGGAGTAGGCCCTGGTTCCTGCTGTCCTGGT | |
| | | GGCTGGGCCCAGCAGGGGCCCTCACTCTTGAAGTCCAGGACTGGGT | |
| | | CTGACCTGGTGGGAGCACCTGCCAGAGGATGCTCTTTCCCAGGACGG | |
| | | ATGGGCCCTATGTCTCAGGAGTGGGGTTGGGGGACAGCCTTCAGCA | |
| | | GCAGCTCACACCCTACCTTCCCCA | |
| IL18BP | rs949324 | CAGCCAGTGGGTGCAGGCTAGTATCGCAAGTTCTCATCTGGCCATGT | (SEQ ID NO: 88) |
| | | GACTGTGTCTCTTTCAGGCAAAGCACTAAAGGGCCAGTACCAGTGA | |
| | | GTGGCCCCACCTGTGTCCCCGATGCTGAC[C/G]TCACCTGGTCCTCCG | |
| | | CCTACTGTCCCTCTCAGTGCCTTCTCTCAGCTCCCAGGCCAACAGTA | |
| | | GCCAAACCCCTAGAGACAGTGATGCCTGCCCGCACCCTGGCCTGGTC | |
| | | CCTGGTCCTTTCACTGGCGCCTTCTCGGAGCTGGCCCAGGGGGCCTGG | |
| | | AGCATGGACAGTGTGGGCGCTCTCCCTACCTTGCCCTCCTTTTTTCTTA | |
| | | AAGCAAAGTCACTTCTCCATCACAACCAGATTTGAGGCTGGTTTTGA | |
| | | TGGCTGGGTCCTTGGGCCTGGCCAGTCTTCCTCTTAGCCTCTGGATCT | |
| | | AGAAGGGACCATAAGAGGAGTAGGCCCTGGTTCCTGCTGTCCTGGT | |
| | | GGCTGGGCCCAGCAGGGGCCCTCACTCTTGAAGTCCAGGACTGGGT | |
| | | CTGACCTGGTGGGAGCACCTGCCAGAGGATGCTCTTTCCCAGGACGG | |
| | | ATGGGCCCTATGTCTCAGGAGTGGGGTTGGGGGACAGCCTTCAGCA | |
| | | GCAGCTCACACCCTACCTTCCCCA | |
| NR3C1 | GRLd21 | CTCCCTTCAGAATTCAGAGGCAGGGAGCAATTCCAGTTTCACCTAAG | (SEQ ID NO: 89) |
| | | TCTCATAATTTTAGTTCCCTTTTAAAAACCCTGAAAACTACATCACCA | |
| | | TGGAATGAAAAATATTGTTATACAATACATTGATCTGTCAAACTTCC | |
| | | AGAACCATGGTAGGCTTCAGTGAGATTTCCATCTTGGCTGGTCACTC | |
| | | CCTGACTGTAGCTGTAGGTGAATGTGTTTTT[G/T]TGTGTGTGTGTCT | |
| | | GGTTTTAGTGTCAGAAGGGAAATAAAAGTGTAAGGAGGACACTTTA | |
| | | AACCCTTTGGGTGGAGTTTCGTAATTTCCCAGACTATTTTCAAGCAA | |
| | | CCTGGTCCACCCAGGATTAGTGACCAGGTTTTCAGGAAAGGATTTGC | |
| | | TTCTCTCTAGAAAATGTCTGAAAGGATTTTATTTTCTGATGAAAGGC | |
| | | TGTATGAAAATACCCTCCTCAAATAACTTGCTTAACTACATATAGAT | |
| | | TCAAGTGTGTCAATATTCTATTTTGTATATTAAATGCTATATAATGGG | |
| | | GACAAATCTATATTATACTGTGTATGGCAT | |
| NR3C1 | rs1438732 | CTTGGCAACAGAATTTATTGGCATATTTGTTGTCCTTCTTAGTGCAGT | (SEQ ID NO: 90) |
| | | GGTTCTAAATGTAGGTGATCATCCAAAATTTTTAGGGACTTTCAAAA | |
| | | ACTCA[C/G]ACTCTTGGGTTCTGACCCTGTAACTCTTAAATGGGGGTA | |
| | | AGAGCAGGGAGGGAAGATGATGTACAGAAATCCGTATTTTTCTTACT | |
| | | GCATTCCAGGTGTA | |
| NR3C1 | rs1866388 | CAGAGAAAACAAAAGATTGCTTATAAAGCTTATAAAAGAGGGCCCC | (SEQ ID NO: 91) |
| | | GATATGCAAAAGCTTGATATTAAGACAAATATTTAACAAATCCTTAA | |
| | | TTATTTG[A/G]CTTAAATTTGCAAAGTAAGACTGAAAAATAAGACCT | |
| | | GATCCATTCAAGAATTTGCTAGGTGCTTTGGTGTAATAAATCTACTT | |
| | | ATAAACATCAGTAATTG | |
| NR3C1 | rs33388 | ACCATGCATAAAGCTAGATATGTCTACTTAGTTTTGTTGCAAGCAAA | (SEQ ID NO: 92) |
| | | GCAATTGCTACAAGGAGGATTATGGGTGAAAGTCATGGATGGATTA | |
| | | TGAGTTA[A/T]TCACACACCTAGAGAAGCATGTAAAATGTGCAGGTA | |
| | | AATTACACCCATTCATTCAGGCAGACGTTTCCTGGCACCTGAATGAA | |
| | | AGGCAAGCACTGTGAGG | |
| NR3C1 | rs33389 | CAAAGATGGAGAAAAACAGAATAAGTCTTGAGACCCCAAGCACTCC | (SEQ ID NO: 93) |
| | | CTTCTCAGGCTGTGTGTTATCAGTTCTGTTTGCTCAGGCTTGCATTAG | |
| | | GGGATG[C/T]GAGTTTTAAGCAGAAGCAATAATAGTACATTGAATGG | |
| | | TAAACAGAATACCAAGAACCAGTATGGTAAGAAGAAAGACTAAAA | |
| | | ACTTTTTGATCGCATAGT | |
| NR3C1 | rs6191 | GAAAATAGTCTGGGAAATTACGAAACTCCACCCAAAGGGTTTAAAG | (SEQ ID NO: 94) |
| | | TGTCCTCCTTACACTTTTATTTCCCTTCTGACACTAAAACCAGACACA | |
| | | CACACA[A/C]AAAAACACATTCACCTACAGCTACAGTCAGGGAGTGA | |
| | | CCAGCCAAGATGGAAATCTCACTGAAGGCTACCATGGTTCTGGAAG | |
| | | TTTGACAGATCAATGTA | |
| NR3C1 | rs6192 | ATCACTTTTGTTTCTGTCTCTCCCATATACAGTCCCATTGAGAGTGAA | (SEQ ID NO: 95) |
| | | ACTGCTTTGGACAGATCTGGCTGCTGCGCATTGCTTACTGAGCCTTTT | |
| | | GGAA[A/C]ATCAACCAAAAGTCTTCGCTGCTTGGAGTCTGATTGAGA | |
| | | AGCGACAGCCAGTGAGGGTGAAGACGCAGAAACCTTCACAGTAGCT | |
| | | CCTCCTCTTAGGGTT | |
| NR3C1 | rs6194 | ACAGCAGGTTTGCACTTGATTGTCTATATGATCTCCACCCCAGAGCA | (SEQ ID NO: 96) |
| | | AATGCCATAAGAAACATCCAGGAGTACTGCAGTAGGGTCATTTGGT | |
| | | CATCCAG[A/G]TGTAAGTTCCTGAAACCTGAATTAAGAGAAATAAAG | |
| | | GTATGAGGCAACACTCTTCAGAAGATCATCTCTGTGGGAATTGCCAA | |
| | | GGAGCAATGAGATCAAT | |
| NR3C1 | rs6195 | TTACCTTGAATAGCCATTAGAAAAAACTGTTCGACCAGGGAAGTTCA | (SEQ ID NO: 97) |
| | | GAGTCCCCAGAGAAGTCAAGTTGTCATCTCCAGATCCTTGGCACCTA | |
| | | TTCCAA[C/T]TTTCGGAACCAACGGGAATTGGTGGAATGACATTAAA | |
| | | AATAGGCTTCTGATCCTGCTGTTGAGAAAGGGATGCTGTATTCATGT | |
| | | CATAGTGGTACATCTG | |
| NR3C1 | rs6199 | AGTAAACTGTGCCCAGTTTCTCTTGCTTAATTACCCCAGGGGTGCAG | (SEQ ID NO: 98) |
| | | AGTTCGATGAAATCTTCTTTTTCTGTTTTCACTTGGGGCAGTGTTACA | |
| | | TTACT[A/G]GGGCTTGACAAAACCAGATCTCCATTATCCTTAATTTTG | |
| | | GGTTTAGTGTCCGGTAAAATGAGAGGCTTGCAGTCCTCATTCGAGTT | |
| | | TCCTTCCAAAAGGA | |
| NR3C1 | rs852983 | GGGGCCCGTTGGGTGGGGGGGCGGGACAGCATTAGGAAAAATAGCT | (SEQ ID NO: 99) |
| | | AACGGATGCCGGGCTTAATACCTGATGAGTTGATAGGTACAGCAAA | |
| | | CCACCATG[A/G]CCACATGTTTACCTATGTAACCTGCAAATCCTGCAC | |
| | | ATGTACCCTGGAACTTAAAATAAAAATTATAATTAAAAAAAGAATT | |
| | | GAGTAGCTGCACCAGTAG | |
| NR3C1 | GRLd21 | CTCCCTTCAGAATTCAGAGGCAGGGAGCAATTCCAGTTTCACCTAAG | (SEQ ID NO: 100) |
| | | TCTCATAATTTTAGTTCCCTTTTAAAAACCCTGAAAACTACATCACCA | |
| | | TGGAATGAAAAATATTGTTATACAATACATTGATCTGTCAAACTTCC | |
| | | AGAACCATGGTAGCCTTCAGTGAGATTTTCCATCTTGGCTGGTCACTC | |
| | | CCTGACTGTAGCTGTAGGTGAATGTGTTTTT[G/T]TGTGTGTGTGTCT | |
| | | GGTTTTAGTGTCAGAAGGGAAATAAAAGTGTAAGGAGGACACTTTA | |
| | | AACCCTTTGGGTGGAGTTTCGTAATTTCCCAGACTATTTTCAAGCAA | |
| | | CCTGGTCCACCCAGGATTAGTGACCAGGTTTTCAGGAAAGGATTTGC | |
| | | TTCTCTCTAGAAAATGTCTGAAAGGATTTTATTTTCTGATGAAAGGC | |
| | | TGTATGAAAATACCCTCCTCAAATAACTTGCTTAACTACATATAGAT | |
| | | TCAAGTGTGTCAATATTCTATTTTGTATATTAAATGCTATATAATGGG | |
| | | GACAAATCTATATTATACTGTGTATGGCAT | |
| NR3C1 | rs1803634 | TGTTGGTGTATCCCCCCCCTGTATA[G/T]TTAGGATAGCATTTTTGATT | (SEQ ID NO: 101) |
| | | TATGC | |
| NR3C1 | rs185434 | CAAAACAAGGGCACCCCTAACAGAAAATGGAAATTAAATACTAGGT | (SEQ ID NO: 102) |
| | | AGACACAGTTTTCTTCTGTCATCTGACACTTTAAGACTCACAAACCC | |
| | | TCTTGTGTTTTTTTTTTTTTTTTTTTTTTTAATTGAGACAAAGTCTTGC | |
| | | TCTGTCACCCAGGCTGGAGTGCAATGGTGCCATCTCGGCTCACTGCA | |
| | | ACCTCCGCCTCCCGGGTTCAAGTGATTCTCAGTGCCAGCCTCCCAAG | |
| | | TAGCTAGGAA[G/T]ACAGATTCATACCACCATGCCCGGCTAATTTTTG | |
| | | TAATTTTAGTAGAGATGGGGTTTTGCTATGCTGGCCAGGCTGGTCTC | |
| | | GAACTCCTGACCTCAACTGATCCACCCACCTGGGCTTCCCAAAGTGC | |
| | | TGGGATTACAGGCGTGAGGCATCGCACCCAGCCCCCTAGGTGCTTTT | |
| | | TAATAGCTTTTAATGTAAAAAAAAAAAAAGCCAGTTTTGTCACCTCT | |
| | | GTCTGCTTAAGTAAACATGAACTAAATAGTAAAATAGC | |
| NR3C1 | rs190488 | GTATGCAGGGTATGCAAAGTACCAAACCATTGGGGGAAGAGAATAC | (SEQ ID NO: 103) |
| | | CTAGAAAAACAATCCAAAAGAATGAAAGACATGAGAGGAGGGAGA | |
| | | AAAAAATGCATAAACAAGGGCATGATAACAGGAAGTAACAGATAA | |
| | | GGTACATTAGTACAGCTAAATTCAAACACATCAGTAGTTTAGTTTCA | |
| | | TTAAATATAGAGATGGGGCCAGGTGTAGTGGCTCACACCTATAATCC | |
| | | CAGCACTTTGGGAGGGTGTGGGCAG | |
| NR3C1 | rs258750 | GAAGTAAGTGTCAAACATAAAGCCAAATATAAGAGTTTTCTGGGAC | (SEQ ID NO: 104) |
| | | AAAGTATGTTTTGATTAGTGAATATAATTATATACCAGCAGCGCCCC | |
| | | CACCCCCGCCCCCAGTTTGTGGATGTTGGTGATAGCTTGAGTTCAAC | |
| | | TTATGAACTTCAGTTTTGTAGACATTTTTCCTAAGGCCAATTATGAAA | |
| | | TATCCTTTCACCTAGTCATGTGTATATAAAATCACCATGTTATTACAG | |
| | | AATTTAGTAA[C/T]ACTG | |
| NR3C1 | rs258751 | TAATTGCTGGAATAAACACTGTTGTTGAAGCCTTCTATCTATCTCAGT | (SEQ ID NO: 105) |
| | | ACTAGAATTAAACTCAAGTGCAGAATGGCAGACAAAGTTAACTAAA | |
| | | AATCACTGTATTATTTCATTTGGTCCTCCAAATAGCTTTGTGAGCTAA | |
| | | GGAGGAGAAGGTGTATCATCACCACTTCCATTTTATAGATGAGAAAT | |
| | | CAAGTGATTTACTCAAGGTTAAGTCCTCCAATTCTTTGTTATCCTGCA | |
| | | TTTTCTCTTGGCTGTAGT | |
| NR3C1 | rs258813 | CATTGTAGTATCATACGAAGTATTTTCACTGCCCTAAAAATCCTCTGT | (SEQ ID NO: 106) |
| | | GCTCTGCACATTTATCCCTCCCTTCCTTCTAAACCCTGGCAACCGCTG | |
| | | ATTTTTTTTTGCTGTCTTCACAGTTTTGCCTTTTATGGAATGTTATATA | |
| | | GTTAGAATCACACAGTATGTAGCCCTTTCAGATTAGCTTTTTTCACTT | |
| | | AGTAATATGCATTTAACGTCCCTCCATGTGTTTTCATGGCTTGACAGC | |
| | | TTATTTCTTTTTAGTCCTCAATAGTATTCCATCATCTGGATGTATCAT | |
| | | AGTTTATCACTTCACCTACTGAAGGACATCTTTGTCTTTGCTAGCTAA | |
| | | AAAGTAATCTCACAGAAGTTCGTTTTATAAACTCTTGTTGCCATTTTC | |
| | | TAGATAATGAATCTCTGACAATCCAGCTCCCATGCTATGTTAACCAA | |
| | | TCCCCAATAGTAATTAAGCACACCTTTTCTAGGATGCGCCTTTTTCTC | |
| | | CCATAATTTCTCATACATGCTAATACTCATTAGGAAACTAAAATTTTT | |
| | | AACCAAATAACAGTGTAGT[A/G]GAGAAAATCACTGTAGTTAGCTTT | |
| | | CTTTTTCCATGTCTTCCCATGATCAAAGATCAAAGGAAGGAAGGAGA | |
| | | AA | |
| NR3C1 | rs258814 | TATTATTAGGGCATTCAGATTTAGCTTTAAGCAGTCACAGCAAAATC | (SEQ ID NO: 107) |
| | | TAATCATGCCACATACATTCCTTACATAAAGTGGGATTTATAATTTTT | |
| | | TTTCCTCAACAGATTTACATTAGTTTCATTTTCATTAAGGGATATGTA | |
| | | CTTCCTATTCTTGTGTTCTCATGCTGCTGCCTAAAAGATGGGCAGTCC | |
| | | TCCACCTTTTTCTTTTCTTTTTTTTTTTTTTTTTTTTTTGAGACGAGTCT | |
| | | TACTCTGTCACCCAGGCTCAAGTGCAGTGGTGTGATCTTGGCTCATG | |
| | | GCAACCTCTGCCTCCAGGGTTCAAGTGATTCTCTGCCTCAGCCTCCC | |
| | | GAATAGCTGGGATTACAGGCGCACTCCACCACACTTGGCTAATTTTT | |
| | | TGTATTTTTTAGTAGAGACGGGGTTTTGCCATATTGGCCAGGCTGGTC | |
| | | TTGAACTCCTGACCTCAAGTGATCCACCCACTTTGGC[A/C]TCCCAAA | |
| | | GTGCTGGGATTACAGGTGTGAGCCACCGCACCCAGCCCTCCACCCTT | |
| | | TTTTCTTAGCCCACTATGTTTTCCATACTGCTCTGGTGTCTGTGACAGG | |
| | | CAGATATTGCATATCAGAAAGTATGCATTCAAGTTCTGACCCTCTAT | |
| | | AGAGCTGTCAAACAGTCTCTCATGGTTGCCCTTAGGTCAGAACGTTG | |
| | | TGGGGGAAAAAAAAATTGTTGTTGTTTTTACAGCCAACAAGAATGA | |
| | | G | |
| NR3C1 | rs33391 | AGCTATCATATCCTGCATATAACACTTCAGGTTCAATAACCTCCAAC | (SEQ ID NO: 108) |
| | | AGTGACACCAGGGTAGGGGTGAGTTGTGGTAACGTTGCAGGAACTA | |
| | | TTGTTTT[C/G]TTACCAGGATTTTCAGAGGTTTCTTGTGAGACTCCTGT | |
| | | AGTGGCCTGCTGAATTCCTTTTATTTTTTTCTTTGTTTTTCGAGCTGTG | |
| | | GGTATTTAAAAAA | |
| NR3C1 | rs6187 | AATTTATTAAAATGATTGTAAAATAGCTTGTATAGTGTAAAATAAGA | (SEQ ID NO: 109) |
| | | ATGATTTTTAGATGAGATTGTTTTATCATGACATGTTATATATTTTTT | |
| | | GTAGG[G/T]GTCAAAGAAATGCTGATGGATAACCTATATGATTTATA | |
| | | GTTTGTACATGCATTCATACAGGCAGCGATGGTCTCAGAAACCAAAC | |
| | | AGTTTGCTCTAGGGG | |
| NR3C1 | rs6188 | AGGCAAAATTAATTGGGAATAGGTTCCTCTGGATCTTTTGCTTTCAG | (SEQ ID NO: 110) |
| | | AAAAAAAAAAGTTTTTTCTCCTTTTCCATGTCACTTTATCATAATTGC | |
| | | TAAATAAAATATTTCTCCCATCTTAATAGTTTTAGAAAGTAAAAATA | |
| | | CTTCTTGAATAAACTGTGTAGCGCAGACCTTCCCATTACAGTTCATTT | |
| | | CTATGTATTTT[G/T]TTTAAATACCCACAGCTCGAAAAACAAAGAAAA | |
| | | AAATAAAAGGAATTCAGCAGGCCACTACAGGAGTCTCACAAGAAAC | |
| | | CTCTGAAAATCCTGGTAACAAAACAATAGTTCCTGCAACGTTACCAC | |
| | | AACTCACCCCTACGCTGGTGTCACTGTTGGAGGTTATTGAACCTGAA | |
| | | GTGTTATATGCAGGATATGATAGCTCT | |
| NR3C1 | rs6193 | AGAAGCTTCAGAAGTTTGGCAATAGTTTGCATAGAGGTACCAGCAA | (SEQ ID NO: 111) |
| | | TATGTAAATAGTGCAGAATCTCATAGGTTGCCAATAATACACTAATT | |
| | | CCTTTCT[A/G]TCCTACAACAAGAGTTTATTTCCAAATAAAATGAGGA | |
| | | CATGTTTTTGTTTTCTTTGAATGCTTTTTGAATGTTATTTGTTATTTTC | |
| | | AGTATTTTGGAGAA | |
| NR3C1 | rs6196 | AACTATACAGGGGGGGGATACACCAACAGAAAGTCTAGAAAATTTC | (SEQ ID NO: 112) |
| | | ATCCAGCCAACTGTGAAAAAAAGTATGAAGAGAAAGTTCATCACAC | |
| | | AGACTTTGGGCACTGGTGGTTTAGGTGCCATCCTTCTTTGACTGTGG | |
| | | AGATTTACGTCCACATATTAAGGTTTCTAATTTCTGGGATATATTAACT | |
| | | AATAAATTTCACCATCTACTCTCCCATCACTGAAAAGTGATGACGAC | |
| | | TCAACTGCTTCTGTTGCCAAGTCTTGGCCCTCTATAAACCACATGTA | |
| | | GTGGGTATTTAAAACAAAACAACAGATGAAAACAATAAAAAATAAA | |
| | | ACAACAAAACCTCTACAGGACAAACTGATAGTTTATACAATAAAAG | |
| | | CTATTAATTCGACTTTCTTTAAGGCAACCATTCTTATTAAGGCAGTCA | |
| | | CTTTTGATGAAACAGAAGTTTTTTGATATTTCC[A/G]TTTGAATATTTT | |
| | | GGTATCTGATTGGTGATGATTTCAGCTAACATCTCGGGGAATTCAAT | |
| | | ACTGATGGTCTTATCCAAAAATGTTTGGAAGCAATAGTTAAGGAGAT | |
| | | TTTCAACCACCTGCAAGAGAAGATATGGTAATGATCAGGCTTCCAAA | |
| | | TTGGTCAGTGGGAACATCTCATGTTTGTTGTCCT | |
| NR3C1 | rs6197 | TCTCTAATATGGCAAAAATGGCTAGACACCCATTTTCACATTCCCAT | (SEQ ID NO: 113) |
| | | CTGTCACCAATTGGTTAATCTTTCCTGATGGTACAGGAAAGCTCAGC | |
| | | TACTGA[C/T]TTTTGTGATTTAGAACTGTATGTCAGACATCCATGTTT | |
| | | GTAAAACTACACATCCCTAATGTGTGCCATAGAGTTTAACACAAGTC | |
| | | CTGTGAATTTCTTCA | |
| NR3C1 | rs852974 | TACAGGAGCTCCACAGAAGTGTTCATGAGTCTGTGGTAACACTACTT | (SEQ ID NO: 114) |
| | | CCTTCTTAATGAAAGTAGAGAAATCTTAATTAGCTTAAAACCTAGTT | |
| | | TATAAAAGTTGGTTTACTTGAAAGGATAAAGAAGATTGATAGACCAC | |
| | | TAGCAAGATTAACAAAGAAAAAAAGAGTGAAGACCCAAATAAGCA | |
| | | CAATAAAAAATGTAAAAGATGACATTACAAGAGATTCCACAGAAGT | |
| | | GAAAAAGATCTTCACACTATTACA | |
| NR3C1 | rs852975 | TACAGGAGCTCCACAGAAGTGTTCATGAGTCTGTGGTAACACTACTT | (SEQ ID NO: 115) |
| | | CCTTCTTAATGAAAGTAGAGAAATCTTAATTAGCTTAAAACCTAGTT | |
| | | TATAAAAGTTGGTTACTTGAAAGGATAAACAAGATTGATAGACCAC | |
| | | TAGCAAGATTAACAAAGAAAAAAAGAGTGAAGACCCAAATAAGCA | |
| | | CAATAAAAAATGTAAAAGATGACATTACAAGAGATTCCACAGAAGT | |
| | | GAAAAAGATCTTGAGACTATTTACA | |
| NR3C1 | rs852976 | TACAGGAGCTCCACAGAAGTGTTCATGAGTCTGTGGTAACACTACTTT | (SEQ ID NO: 116) |
| | | CCTTCTTAATGAAAGTAGAGAAATCTTAATTAGCTTAAAACCTAGTT | |
| | | TATAAAAGTTGGTTACUGAAAGGATAAACAAGATTGATAGACCAC | |
| | | TAGCAAGATTAACAAAGAAAAAAAGAGTGAAGACCCAAATAAGCA | |
| | | CAATAAAAAATGTAAAAGATGACATTACAAGAGATTCCACAGAAGT | |
| | | GAAAAAGATCTTCACACTATTACA | |
| NR3C1 | rs852977 | ACTAAAAACTCAAGTGTCTTTTAATACATATTTAAATGGTCAAAGTATA | (SEQ ID NO: 117) |
| | | TTACATACATAGGAGATTAGAGAGCAGCAAGATGAAACTGGGTAAA | |
| | | ATCTGGGCAGAGATCTGGTATCTAAGAAAGTGGGGAATACTGTTTTT | |
| | | ATAACAAAAATAAAGTACCGTTGTGGAACTGAAAGCAAACTTCTGT | |
| | | GTGCATTTTTTTTAGTTAATCTCT[A/G]CAGTTTTTATAACATTTACAAG | |
| | | AAAGTGGGCAGCTATCATTTTATGTAAATCAATGTTTAACATGCTGA | |
| | | CACTGTGCAGTTAAGTTTAAATAGCCTGGTCAAACGTAGATAGAGTT | |
| | | GTGTGTGTGGTTTGGGGAATTAGACTGTTCATAGTCATACCCATAAA | |
| | | TCTATTTTCTATTTAACAAGATGTCTACACACAGTGTGTGCTAGATA | |
| | | GGACCAACAATTAGTCTCTCCTATCAAAAGAACCACATAGGTCAGTT | |
| | | GCAGTGGCTCACACCTGTAATACCAGCACTTTGGGAGGCCACGTTGG | |
| | | GAGGATCACTTAAGGCCAGGAGTTAGAGACCAGCCTGGGAACATAG | |
| | | C | |
| NR3C1 | rs852978 | TTCTTGCCTACATTGCCATTATGAGGTCGAGAGGTCAAAAACAACAA | (SEQ ID NO: 118) |
| | | AAAGAGTCTCCCTTTTTCTGTTCAACAAACTGAAAACAGTGCAATAA | |
| | | AAAACCTTATTGCAGTATTCACATATAAAAAAAGTACACAAATCAGT | |
| | | GAACCATTTAATAAATTTTTACAAACCAAGAACCTTTATGTAATCAG | |
| | | CTCTCAGATCAAGAAAGAGAACAGTACCCCAACAGGCCCCCTTTCTG | |
| | | CCCAATTCCAGCCAGTACCCACCAAGGGTAACTACTTTTTTGATAAA | |
| | | CTGTGTTTTACACAGTTTTGATAGATTATGTTTTTTTATTTTTATTTTTT | |
| | | TGAGATGGAGTCTCGGTCTGTCGCCCAGGCTGGGAGTGTAGTAGTGT | |
| | | AGTGGCATGACCTGACTGCAACCTCTGCCTCCCAGGTTCAAGCAATT | |
| | | CTCCTGGCTCAGACTCCCGAGTAGCTGGGATTACAGGTGCACGCCAC | |
| | | CACGCCTGGCTAATTTTTATATTTTTAGTAGAGACAGGGTTTCACCAT | |
| | | GTTGGCTAGGATTGTCTCGATCTCAGGACCTCGTGATCCACCCGCCT | |
| | | CGGCCTCCCGATGTGCTGGGATTACAGGCATGAGCCACCACGGCCA | |
| | | GCCAATAGATTGTGTTTTAAACAAATAATAGTTTGGGGTAGATGGTT | |
| | | AACTGTATCAGGTTTCAATTCTTTGTAAAGAA[C/T]AGGCCACAAAATT | |
| | | GACCACTAGACTATAATTCACCTTTGTTGAGAGCTGCTCTCCAATGC | |
| | | CAAATATCTGTACCAGCTGTTTCAATTTCTTTTTGTACTCTATAAATA | |
| | | TCCTAGCTTGTTTACTTGGACAGAAAATGGAAGCACTAACCCAATTC | |
| | | TATCCCCTATAAATCAAAAGTACGATCAACAGATACATCTATTGA | |
| NR3C1 | rs852979 | AAGTGGTGACGAAACCTATTAGCTTTATGGAAGTTAAAGCCCAT | (SEQ ID NO: 119) |
| | | GTTTCTAATACAATGAACATTATGTTATGCCCAAACTTAACACCATC | |
| | | ATTTCATATGATAGCACTTTCTTATAGTGTTACCTTATGCTCCCTGAC | |
| | | CAAACTCCCAGACATCAACTTGTACTTTTCTATTTTATTCTAGATCTT | |
| | | TTTGTATTGT[C/T]GTTTTAAATACTTTCCTGCCCATTAGAGGACCTAG | |
| | | GAGCCACCCTCCTCTCCCCTCTTAACTGATATTTAGCCTTTCATGGGC | |
| | | TTTGCATATAATGGAAATTTCAAAATCCACCCTGAGAAATGAAAACC | |
| | | AAGTAGAGGAAAAATAAACTCTTCAAAACACACACTACCTTCCACT | |
| | | GCTCTTTTGAAGAAAACTTTACAG | |
| NR3C1 | rs860457 | GAGTTGTGTGTGTGGTTTGGGGAATTAGACTCTTCATAGTCATACCC | (SEQ ID NO: 120) |
| | | ATAAATCTATTTTCTATTTAACAAGATGTCTACACACAGTGTGTGGT | |
| | | AGATAGCACCAACAATTAGTCTCTCCTATCAAAAGAACCACATAGGT | |
| | | CAGTTGCAGTGGCTCACACCTGTAATACCAGCACTTTGGGAGGCCAC | |
| | | GTTGGGAGGATCACTTAAGGCCAGGAGTTAGACACCAGCCTGGGAA | |
| | | CATAGCAAGACCCCTTTGTCT | |
| NR3C1 | rs864082 | TAGGACTAACCGGCAGGGAAAAAAACTATACGGCAGGGAAAAAAA | (SEQ ID NO: 121) |
| | | CTATAAGCCATCGCTGTT1TACAATTTTGCAATAATTAGATTTTCTGT | |
| | | AGTATAGTAATGTGTAAAATTAACCCATTGTTAATATAGAATGCCGT | |
| | | TATCACTCCTGATTAAGCGGTCTTCATTTTCATGTTAATACTGATGTC | |
| | | TTGTAATGCTTT[A/C]TGGAATCAAACATTTTCATACATATTCATTAG | |
| | | TCTAATTCTAATCATAATCCAATGAAAAAGAGCAGGAAAGATGCTC | |
| | | AAGGAGGTTATATTCAAGTCCACATGGCAAGTAAGAAATAAGACTA | |
| | | CTCGGCTGGGCATGGTGACTTACTGCCTGAAATCCCAGCACTTTGGG | |
| | | AGGCCAAGGTGAGCGGAATTGCTTGAACCTGGGAGGCGGAAGTGGC | |
| | | AGTGAGCTGAGATCATGCCAATGCACTCCAGCCTAGGCAACACAGC | |
| | | AAGACTCTGTCTCGGGAAAAAAATAATAATAATAAGACTTCTAGAA | |
| | | GCTCCTAAATCCATAGCTTTTCCTCTATACCAGCATCTTCTAAAAATG | |
| | | TCAGCAGCAGTGAAGTTTCAGTTTGGGAAATAATGCATTTCCCCTCT | |
| | | CTGGAGAGTGCACAGTTATATCTCCAAGAAGTACTGAAATTCAGAA | |
| | | GTCTGCCTAATATGTATTAAACATTTAGCTTTTCTCAAACTTTGACCA | |
| | | CCAAATCCTTTGTCTCGCTCTAACTATAGTTAACACAGAATCAGTGT | |
| | | TCCCAGGAGCACACTGTGAAAAATGTAGCACTCTACAAAAGTCCTA | |
| | | ATCTCCA | |
| POMC | rs1009388 | CCTGTCCCCGTCCTCGCGATGCAGTCGGCCGGCTCCGGCTCCGAAGG | (SEQ ID NO: 122) |
| | | CGGACCTGGGCGCCTCTGGCTCTCCGCGGTCCCGAGTTCTGGACAAA | |
| | | CTTTCTGCGCCGACTGCGGCATGAGAAGCCGCCAGTAGCTGAGCTGG | |
| | | AGGGCCCACGTCCGGCCCCTGGGCGGACGGCCGCGAAGCTGCAGGC | |
| | | GCTGTCTCCAGGGAGCCGGCGGCCTCCTCTCCCC[C/G]AGGGGCTCG | |
| | | CGGCGGTCCGGAGGCTCCGA | |
| POMC | rs1042571 | GCCCCAGGGCTACCCTCCCCCAGGAGGTCGACCCCAAAGCCCCTTGC | (SEQ ID NO: 123) |
| | | TCT[C/T]CCCTGCCCTGCTGCCGCCTCCCAGCCTGGGGGGTCGTGGCA | |
| | | GATAATCAG | |
| POMC | rs15461 | ACGCTGTTCAAAAACGCCATCATCAAGAAGGCCTACAAGAAGGGCG | (SEQ ID NO: 124) |
| | | AGTGAGGGCAGAGCGGGCCCCAGGGCTACCCTCCCCCAGGAGGTCG | |
| | | ACCCCAAA[A/G]CCCCTTGCTCTCCCCTGCCCTGCTGCCGCCTCCCAG | |
| | | CCTGGGGGGTCGTGGGACATAATCAGCCTCTTAAAGCTGCCTGTAGT | |
| | | TAGGAAATAAAACCTTT | |
| POMC | rs1561287 | TCTTTAAACTGAGTATGGGCCAGGCATGGTGGCTCACACCTGTAATCC | (SEQ ID NO: 125) |
| | | CAGCACTTTGGGAGGCCAAGGTGGCAGGATCCCTTGAGCCCAGAAG | |
| | | TTTGAGA[C/G]CAGCCTGGGCAACATAGTGAGACCTCATCTCTATTA | |
| | | AAAAAAAAAAAAAAAACTCAACAGTATTATGGACATGTGCGACACA | |
| | | CACCCCTTTTTCTTCTTG | |
| POMC | rs1866146 | TAGCATTGGTCAGGACTCTGCTGACAGCACTGAGTAGGCAGGAATG | (SEQ ID NO: 126) |
| | | TATTTGAGATTTGGAAGTACTGTTAATTTGGTGGAGTCAGGTGAATG | |
| | | GATAAGA[A/G]GCAGATCGGCAGAAAGCATCAGTGTGGTCCCGAGG | |
| | | CTCCCTCTGTTTTCCTTGCCATGGAGTCCCCAGCGCCTTGTGCCTGTT | |
| | | TCCATTTCCCTCTCTCT | |
| POMC | rs2028195 | TTGAGCCTGGGACACGGAGGTTGCAGTGAGCTGAGATCACGCCACT | (SEQ ID NO: 127) |
| | | GCACTCCAGCCTGGGCAACAGAGTAAGACTCTGTCTCAGAAAAAAA | |
| | | AAAAAAAA[G/T]TTAACCAGCAGCCCTCCAGGTCGCTCTGCCTATGG | |
| | | AGTAGCCATTCTTTCATTCCTTTACTTTCCTAATAAATAAACTTGCTT | |
| | | TCACTTTAATCTATGGA | |
| POMC | rs2028196 | GCCTGTCCAAGATGGTGAAACTCTGTCTCTACTAAAAATACAAAAAT | (SEQ ID NO: 128) |
| | | TAGCCGGGCGTGGTGGCAGACGCCTGTAATCCCAGCTACTTGGGAG | |
| | | GCTGAGG[C/T]AGAGAATTGCTTGAGCCTGGGAGACGGAGGTTGCAG | |
| | | TGAGCTGAGATCACGCCACTGCACTCCAGCCTGGGCAACAGAGTAA | |
| | | GACTCTGTCTCAGAAAAA | |
| POMC | rs2071345 | CCCAGCGGAAGTGCTCCATCCTGTAGGGGCCCTCGTCCTTCTTCTCG | (SEQ ID NO: 129) |
| | | GCCGCCACCAGCAGGCTGTGCTCCAGGTCGGCCTGGGCCCCTGCGCC | |
| | | GTCATC[A/G]GCAGGGCCGTCGGGGCCATCTCCCTCCCGGAGTCGCT | |
| | | GGCCAGTCAGCTCCCTCTTGAACTCCAGGGGGAAGGCCTCGGCCGA | |
| | | CTCGTCCTCGGCGCCGT | |
| POMC | rs934778 | TTGCATTTGGTGACATACGTAACTACCATTTTTCTGTGACTGTAACAT | (SEQ ID NO: 130) |
| | | CTGGGCATTTTTCAGAGCTAAATGTGCTATGGTCAACTTGGAGCTTT | |
| | | AATCTAATTGCCTGGTCCACCAAGTTCTGGCTGTGTACTTGAATAGA | |
| | | TCAC[C/T]GGCAGGGTACAATGGGAACAGCCTGTCCCTTGGAGCCAG | |
| | | GAGAGGACACCAAGGTTGACCAAAGCTCGTTCAGTTGCCCCTTTAGC | |
| | | CGAAGCGCACCTGGGCCAGTCACTGGCTGCCAGTGCCATCTAATGGC | |
| | | TGCTCTGAAAATGCTCAGCCTTGCCCGGCAACCCTTCAGAAGCTAGC | |
| | | ACCGTGCAGGCCCAGCGCCTGGGGAATAGGGCGAGGGTGGGGTAGA | |
| | | GAGAAGGAAGTGGCCTCCTGAAGTAGAAATCAGCGCTTCAGAGGAC | |
| | | TTTCACTTCCAAAGCCTCCCCTATATAAAAAAGATTTGGCCCACGCC | |
| | | TCCCCAAATGAGAGATTTATTTTAGGCAAACTTATTTTAAAATGCCA | |
| | | GCGTTCATTAGGAGTGACAAGACACTTAGTCATCCACGCTTTAATGT | |
| | | GAATT | |
| STAT3 | G3363a12 | ATCTCTACAGAAATTTTTTTTAAAAACTAGCTGATTGTGGTGGCATG | (SEQ ID NO: 131) |
| | | CACCTGTAGTCCCAGCTACTCAGAAGGCTGAGGTGAGAAAATTGTTT | |
| | | GAGCCTGGGAGGTCGAAGCTGCAATAAGCCGTGATTGCGCCACTGC | |
| | | ACTCCAGCCTGGCGGACAGAGTGAGAGCCAGTCTCAAAAAAAAAAA | |
| | | AAAAAGACTCAGGCTAATGTGCCTTCTGTTACAGAAATAGTAACGA | |
| | | CCTCCCCTTCGCCCCCCGCCGACA[G/A]AGAGCCTTCACCCAGGCTCT | |
| | | GAAGCCTTTGTTCCGTTGTTTCCTAGAATAAATGCTTTCCTTGATGAA | |
| | | TACATTAGTTTTAAGGTGCCACAGTTCAGTCCACATCTCCATGGTCT | |
| | | GCTGCTGATTTTTATTCTCTTTCTCTCCTACTTATAGAGCAGGTATCT | |
| | | TGAGAAGCGAATGGAGATTGCCCGGATTGTGGCCCGGTGCCTGTGG | |
| | | GAAGAATCACGCCTTCTACAGACTGCAGCCACTGCGGCCCAGGTGA | |
| | | GACCTGAGACAAAACAAATCCCTGGTCTGGGAGG | |
| STAT3 | G3363a14 | AGGTCCAGGAGTATTCCCTCAGGTCCAGGAGTATTCCCTCAGGTCAA | (SEQ ID NO: 132) |
| | | GGAGTATTCCCTCAGGTCAAGGAGTATTCCCTCAGGTCAAGGAGTTT | |
| | | TTTCTTCCTTCGCAGACATGCAAGATCTGAATGGAAACAACCAGTCA | |
| | | GTGACCAGGCAGAAGATGCAGCAGCTGGAACAGATGCTCACTGCGC | |
| | | TGGACCAGATGCGGAGAGTAAGGGCATAGGTCGGACCAC[T/A]TCCC | |
| | | CCATGTGTCTCGCTCACTTGCGGGATTTCAGCGTCTTGTGGCAGAAC | |
| | | TTTGCTTGGTTTTCTAAGAAGTTGCTGCTCTGGAGTTGACTAAAGAATG | |
| | | TGGTTAGAGACAGTCTGAGGAAATGTTTTCTGACTTTGTTTTGGTTTC | |
| | | CAACCAGAGCATCGTGAGTGAGCTGGCGGGGCTTTTGTCAGCGATG | |
| | | GAGTACGTGCAGAAAACTCTCACGGACGAGGAGCTGGCTGACTGGA | |
| | | AGAGGCGGCAACAGATTGCCTGCATTGGAGG | |
| STAT3 | G3363a16 | TGTTGGGCAATGGCTACTTCTAGATTGTTTACCCCTACTGGGACTTGT | (SEQ ID NO: 133) |
| | | GGTGAACATATGCACACTTTGGTTTACAGTTGGGACCCCTGATTTTA | |
| | | GCAGGATGGCCCAATGGAATCAGCTACAGCAGCTTGACACACGGTA | |
| | | CCTGGAGCAGCTCCATCAGCTCTACAGTGACAGCTTCCCAATGGAGC | |
| | | TGCGGCAGTTTCTGGCCCCTTGGATTGAGAGTCAAGATTGGTAAGTC | |
| | | CTTCTTTAAGTGACTCTCCAAATTGTTAGGTTTCAGTTTGAGTCAAGA | |
| | | GACATGAACTCTTAATGTCATGCCTTGCTGTTCCATTAAAAAATGTA | |
| | | TGGGTACAGGTGATGGGGAAAATGAGATCAGGAGATAAAG[G/T]GG | |
| | | CACCCTTTGGTCTTGTAAAGCCTTTTTTATCTTAGAAGGGCATGTGGG | |
| | | CAACTGTCTTTGACACAUGAAACCGCCTGTATGGTGGTGGATGTCT | |
| | | TGAAGGTTGATTTGGACCTCATTTACTTGGGCAGATCCTCTATATATT | |
| | | CTGATAATCCAGTGATGTGGTAGACATATTTTTTCTCTGAATGTGAA | |
| | | TTCTGTCATAGCTAGAACTTTGGGTTGATACTTGTAATTCCCCTTTAG | |
| | | TTAAAGGAAGGAGCCACAGGGGTGTATTAGTCTGTTCTCAATTTGCT | |
| | | ATAAAGAAATACCTGAGACTGGGTAATTTATAAGAAAAG | |
| STAT3 | G3363a3 | GGGCTCTCACTTTGTTGCCCAGACTGTTATGGAACTCCTGGGCTCAA | (SEQ ID NO: 134) |
| | | GGGATCCTCCCAGCTTGGCCTCCCACAGTGCTGAGATTATAGATGTG | |
| | | AGCCTGTAATTATAGACAGCTTGGCCTATTTACCTGTTGGAAATGAA | |
| | | GAATTATGAATTTTACATTTCTTCAAGAAAAGGTTATGGGAGAGTTA | |
| | | CTGACTTTTTTTCCTTGGATTTTTTCTTTTTAAATAGGTTGCTGGTCAA | |
| | | ATTCCCTGAGTTGAATTATCAGCTTAAAATTAAAGTGTGCATTGACA | |
| | | AGTAAGTACTCCTATCTTAGCTGTNTTTTTCAAATGAGGAATAGAAA | |
| | | AATGAGAACTTTGACAGACATCATTTGAACTAGAGACTCT[G/A]TCTT | |
| | | TATTCAGAGATCTTCATTTTGTGGACAAAAGTTTTCAAAAGCCTTGG | |
| | | GGTGCATTGTCATTTACGTGTCTGAACAAAGCCACAAAGCTGGGGGT | |
| | | ACAGATTTTGATTTGTGGTTGCTATTGTGACAACCAGTCCCTCTTTTCC | |
| | | TTGTTTTAGTTTTTTACTTGTACATGTCATTCATGCATATTATATATAA | |
| | | GACTGAGATCATGTGTTAATTAACGACTGGGATACGTTCTGCAAAAT | |
| | | GTATCATTAGGCAATTTTGTTGTGCAAATGTTGTAGAGTATATAGTC | |
| | | CTTACACAAACCTGGGTGGCAGAACCTACTGCACAC | |
| STAT3 | G3363a4 | AGTAAATAACAGGTGGTCAAAGTAGGCTTTTTGAAGAAACACAGAG | (SEQ ID NO: 135) |
| | | CCTATTTTATTAACAACAGTCTGTGTTCTTACAGAGACTCTGGGGAC | |
| | | GTTGCAGCTCTCAGAGGGTAAGTTCAGCCTAGAGGCTTCCTTTTGTT | |
| | | CCGTTTAACCTAACTTCATCCTCCGGCTACTTGGTCACCTACATAGTT | |
| | | GATTGTTCCCCTGTGATCAGATCCCGGAAATTTAACATTCTGGGCA | |
| | | CAAACACAAAAGTGATGAACATGGAAGAATCCAACAACGGCAGCCT | |
| | | CTCTGCAGAATTCAAACACTTGGTATGTGGGAGGAGCTCCCCTTCAC | |
| | | AAAGGGCCTCTGGCTGC[C/G]GGAGAGGGCTAGGGAGAGCCTCACA | |
| | | GGACACCTGCCTTTTTCTTTTCTTACAGACCCTGAGGGAGCAGAGAT | |
| | | GTGGGAATGGGGGCCGAGCCAATTGTGATGTAAGTTTTGTTGGGGAT | |
| | | GAAAGACAACTGGGGTGTTTTCCTTGAGGGAGAGAGGGGTAAAGAT | |
| | | CCTTCTTAATCCCCAGAATTAGAAACATCAACCTGTTCTTTCAGCTGT | |
| | | AGTTATTCCAAAAAGTCACTTCAGGCCAAAGTGACATGAACAGAAG | |
| | | TTCCATGTGCCATGGAGCTCTCTGGCTTGGAACATTTCCGT | |
| STAT3 | rs1026916 | CAGGAGAGATAAATAGTAGGTAACTAGCTGTCCCTGGGAAAGAGTC | (SEQ ID NO: 136) |
| | | CCAAGGATAAGGTGCGGACTAACTTTCCACTTACTACATATCTTTTG | |
| | | TACTGATTCATTTCAAAATTTACTCTAAAATATTGGTATTACAATAA | |
| | | AGAAAACTGGAGTCTAGAACTGAATGACAAAACTGATACATTCTTA | |
| | | CTACAAATCTGTGGTTAAGATTAGCATTAATCTTTCCTAGGCAAAGA | |
| | | GGAAAAAGTTTAACCCAAAGAC | |
| STAT3 | rs1064112 | AATCCCAAGAATGTAAACTTTTTTACCAAGCCCCCAATTGGAACCTG | (SEQ ID NO: 137) |
| | | GGA[C/T]CAAGTGGCCGAGGTCCTGAGCTGGCAGTTCTCCTCCACCA | |
| | | CCAAGCGAGG | |
| STAT3 | rs1064113 | AACTTTTTTTTACGAAGCCCCCAATTGGAACCTGGGATCAAGTGGCCGA | (SEQ ID NO: 138) |
| | | GGT[C/G]CTGAGCTGGCAGTTCTCCTCCACCACCAAGCGAGGACTGA | |
| | | GCATCGAGCA | |
| STAT3 | rs1064114 | TTTTTTACCAAGCCCCCAATTGGAACCTGGGATCAAGTGGCCGAGGT | (SEQ ID NO: 139) |
| | | CCT[C/G]AGCTGGCAGTTCTCCTCCACCACCAAGCGAGGACTGAGCA | |
| | | TCGAGCAGCT | |
| STAT3 | rs1064115 | CCAATTGGAACCTGGGATCAAGTGGCCGAGGTCCTGAGCTGGCAGT | (SEQ ID NO: 140) |
| | | TCTC[C/G]TCCACCACCAAGCGAGGACTGAGCATCGAGCAGCTGACT | |
| | | ACACTGGCAGA | |
| STAT3 | rs1064116 | CACATGGGCTAAATTTTGCAAAGAAAACATGGCTGGCAAGGGCTTC | (SEQ ID NO: 141) |
| | | TCCT[A/T]CTGGGTCTGGCTGGACAATATCATTGACCTTGTGAAAAAG | |
| | | TACATCCTGG | |
| STAT3 | rs1064117 | TTTTGCAAAGAAAACATGGCTGGCAAGGGCTTCTCCTTCTGGGTCTG | (SEQ ID NO: 142) |
| | | GCT[A/G]GACAATATCATTGACCTTGTGAAAAAGTACATCCTGGCCC | |
| | | TTTGGAACGA | |
| STAT3 | rs1064118 | AACATGGCTGGCAAGGGCTTCTCCTTCTGGGTCTGGCTGGACAATAT | (SEQ ID NO: 143) |
| | | CAT[C/T]GACCTTGTGAAAAAGTACATCCTGGCCCTTTGGAACGAAG | |
| | | GGTACATCAT | |
| STAT3 | rs1064119 | TTCTCCTTCTGGGTCTGGCTGGACAATATCATTGACCTTGTGAAAAA | (SEQ ID NO: 144) |
| | | GTA[C/T]ATCCTGGCCCTTTGGAACGAAGGGTACATCATGGGCTTTAT | |
| | | CAGTAAGGA | |
| STAT3 | rs1064120 | GAGCGGGAGCGGGCCATCTTGAGCACTAAGCCTCCAGGCACCTTCCT | (SEQ ID NO: 145) |
| | | GCT[A/G]AGATTCAGTGAAAGCAGCAAAGAAGGAGGCGTCACTTTCA | |
| | | CTTGGGTGGA | |
| STAT3 | rs1064121 | AGCGGGAGCGGGCCATCTTGAGCACTAAGCCTCCAGGCACCTTCCTG | (SEQ ID NO: 146) |
| | | CTA[A/C]GATTCAGTGAAAGCAGCAAAGAAGGAGGCGTCACTTTCAC | |
| | | TTGGGTGGAG | |
| STAT3 | rs1064122 | CGGGAGCGGGCCATCTTGAGCACTAAGCCTCCAGGCACCTTCCTGCT | (SEQ ID NO: 147) |
| | | AAG[A/C]TTCAGTGAAAGCAGCAAAGAAGGAGGCGTCACTTTCACTT | |
| | | GGGTGGAGAA | |
| STAT3 | rs1222186 | ACTATACCTGCTCCATCATAGATTAACACTGGGGTCATGCATGAATA | (SEQ ID NO: 148) |
| | | TTTGCATTTTGAAACTAACATATCCTAAAAACGTGTAAGTTATTGAA | |
| | | AATTATCTAATTCATTTCCAGTACATAACTAAGGATTTTTTGGTTGTT | |
| | | GCTGTTCTTATTTTTAGCTGGGGACAGAGGAGGGCTAGTAAGTAGTA | |
| | | CAATAATTGTGTTCTTTTTTTTTTTTTTTTTTTTGAGATGAAATACTGT | |
| | | CACCCGGGCTGGTGTGC | |
| STAT3 | rs1231903 | ACTATACCTGCTCCATCATAGATTAACACTGGGGTCATGCATGAATA | (SEQ ID NO: 149) |
| | | TTTGCATTTTCAAACTAACATATCCTAAAAACGTGTAAGTTATTGAA | |
| | | AATTATCTAATTCATTTCCAGTACATAACTAAGGATTTTTTGGTTGTT | |
| | | GCTGTTCTTATTTTTT7AGCTGGGGACAGAGGAGGGCTAGTAAGTAGTA | |
| | | CAATAATTGTGTTCTTTTTTTTTTTTTTTTTTTTGAGATGAAATACTGT | |
| | | CACCCGGGCTGGTGTGC | |
| STAT3 | rs1236315 | ACTATACCTGCTCCATCATAGATTAACACTGGGGTCATGCATGAATA | (SEQ ID NO: 150) |
| | | TTTGCATTTTCAAACTAACATATCCTAAAAACGTGTAAGTTATTGAA | |
| | | AATTATCTAATTCATTTCCAGTACATAACTAAGGATTTTTTGGTTGTT | |
| | | GCTGTTCTTATTTTTAGCTGGGGACAGAGGAGGGCTAGTAAGTAGTA | |
| | | CAATAATTGTGTTCTTTTTTTTTTTTTTTTTTTTGAGATGAAATACTGT | |
| | | CACCCGGGCTGGTGTGC | |
| STAT3 | rs1803125 | TGACAGCTTCCCAATGGAGCTGCGG[A/C]AGTTTCTGGCCCCTTGGAT | (SEQ ID NO: 151) |
| | | TGAGAG | |
| STAT3 | rs1803126 | CCTACTTCTGCTATCTTGAGCAAT[C/T]TGGGGACTTTTAAAAATAG | (SEQ ID NO: 152) |
| | | AGAAAT | |
| STAT3 | rs1905340 | TATAAAGTTAAACTGTTACAGAATTGTTTTTCTTAAACTGCAATAAT | (SEQ IDNO: 153) |
| | | GAGACTTTAGCACTCTCTTGTCCTCTAAAAGACATTATTTCATGACAT | |
| | | GTGCCCATTGGCAGTATTTTGAGAATCTAAGAAAGTAGATCA[A/C]AC | |
| | | TAAATATTGATATGCAGACACTAAAATCGTACAACGACTTGGATGAC | |
| | | TAGGTTTGAGATATTCCCAAAGTGAGAGGTTTTTGTTTTGTTTTGTTT | |
| | | TGTTTTTTTGAGACAAGGTCTCGCCCTGTCGCCCAGGCTGGAATGCAG | |
| | | TGGTGCAATGTCAGCTCACTGCAACCTCTGCCTCCTGAGTTCAAGCA | |
| | | ATTTCTCCTGCCTCAGCCTCCCTGGTAGCTGGGACTGCAGGCATGCAC | |
| | | CACCACACCTGGCTAATTTTTTGAATTTTTAGTAGAGACAGGGTTTCT | |
| STAT3 | rs1905341 | TATAAAGTTAAACTGTTACAGAATTGTTTTTCTTAAACTGCAATAAT | (SEQ ID NO: 154) |
| | | GAGACTTTAGCACTCTCTTGTCCTCTAAAAGACATTATTTCATGACAT | |
| | | GTGCCCATTGGCAGTATTTGAGAATCTAAGAAAGTAGATCACACTAA | |
| | | ATATTGATATGCAGACACTAAAATCGTACAACCACTTGGATGACTAG | |
| | | GTTTGAGATATTCCCAAAGTGACAGGTTTTTGTTTTGTTTTGTTTTGT | |
| | | TTTTTGAGACAAGGTCTCGCCCTGTCGCCCAGGCTGGAATGCAGTGG | |
| | | TGCAATCTCAGCTCACTGCAACCTCTGCGTCCTGAGTTCAAGCAATT | |
| | | CTCCTGCGTCAG[C/T]GTCCCTGGTAGCTGGGACTGCAGGCATGCACC | |
| | | ACCACACCTGGCTAATTTTTGAATTTTTAGTAGAGACAGGGTTTCT | |
| STAT3 | rs1963987 | TGGGATTACAGGCGTAAGCTACCACGCCTGGCCTGGGATCAGGTTTT | (SEQ ID NO: 155) |
| | | CTGACAGAACCTGAGAGGGCTGCACTTCTCCCTCCCTCTTTGGGGGA | |
| | | CAGACTCAGAATACCCCTCTTGCTACTGTGAAGACGGCTGGTGGAGC | |
| | | TCTCAAGCATATATTCAGGGAAGTGCAGGTAGTACCTCCCAGCAGTC | |
| | | TTTACATTTGAATAATTAATAATGTAAGGGAGCAGCATCCAACAGAAA | |
| | | TAGAATACAGGCCACACATGCATTTTAAATTTTTCTGGTAGCCATACT | |
| | | TAAAAAGTTAAAAGAGGCTGGGTGCAGTGGCTCA[C/T]GCCTGTAAT | |
| | | CCCAGAACTTTGGGAGGCCAAGGCAGGCGGATCATGAGGTCAGGAG | |
| | | ATCGAGAGCATCCTGGGCAATATGGTGAAACCCCGTCTGTACTAAAA | |
| | | ATACAAAAATTAGCTGGGTGTGGTGGCACATGCCTTTAATCCCAGCT | |
| | | ACTAGGGAGGCTGAGGCAGAAGAATCGCTTGAACCCAGGAGGAGG | |
| | | AGGTTGGAGTGAGCCGAGATCGTGCCACTGCACTCCAGCCTGATGAC | |
| | | ATAGCGAGACTCCATCTCAAAAAAAAAAAAAAAAAAGAAAAGAAA | |
| | | AGAAACAGGTGAAATTAATTTTAATG | |
| STAT3 | rs1963988 | TGGGATTACAGGCGTAAGCTACCACGCCTGGCCTGGGATCAGGTTTT | (SEQ ID NO: 156) |
| | | CTGACAGAACCTGAGAGGGCTGCACTTCTCCCTCCCTCTTTGGGGGA | |
| | | CAGACTCAGAATACCCCTCTTGCTACTGTGAAGACGGCTGGTGGAGC | |
| | | TCTCAAGCATATATTCAGGGAAGTGCAGGTAGTACCTCCCAGCAGTC | |
| | | TTTACATTGAATAATTAATAATCTAAGGCAGCAGCATCCAACAGAAA | |
| | | TAGAATACAGGCCACACATGCATTTTAAATTTTCTGGTAGCCATACT | |
| | | TAAAAAGTTAAAAGAGGCTGGGTGCAGTGGCTCACGCCTGTAATCC | |
| | | CAGAACTTTGGGAGGCCAAGGCAGGCGGATCATGAGGTCAGGAGAT | |
| | | CGAGACCATCCTGGCCAATATGGTGAAA[C/T]CCCGTCTCTACTAAA | |
| | | AATACAAAAATTAGCTGGGTGTGGTGGGACATGCCTTTAATCCCAGC | |
| | | TACTAGGGAGGCTGAGGCAGAAGAATCGCTTGAACCCAGGAGGAGG | |
| | | AGGTTGCAGTGAGCCGAGATCGTGCGACTGCACTCCAGCCTGATGAG | |
| | | ATAGCGAGACTCCATCTCAAAAAAAAAAAAAAAAAAGAAAAGAAA | |
| | | AGAAACAGGTGAAATTAATTTTAATG | |
| STAT3 | rs2230097 | GCGGCGTGTGGAGGAGCTCCTGGGCCGGCCAATGGACAGTCAGTGG | (SEQ ID NO: 157) |
| | | ATCCCGCACGCACAATCGTGACCCCGCGACCTCTCCATCTTCAGCTT | |
| | | GTTCATC[C/T]TCACCAGAGGAATCACTCTTGTGGATGTTTTAATTCC | |
| | | ATGAATCGCTTCTCTTTTGAAACAATACTCATAATGTGAAGTGTTAA | |
| | | TACTAGTTGTGACCTT | |
| STAT3 | rs2354155 | TGGGGTTTGACCGTGTTAGCCAGGATGGTCTCAATCTCCTGACTTTGT | (SEQ ID NO: 158) |
| | | GATTCACCCACCTTGGCCTCCCAAAGTGCTGGCATTACAGGCGTGAG | |
| | | CCACC[A/G]CTCCCGGCCTTTTTTGTTTTTTGAAACCAAGTGTCGCCC | |
| | | TGTCGCCCAGTCTGGAGTGCAATGGCACGATCTTGGCTCACTGCAAC | |
| | | CTCCGCCTCCTGCA | |
| STAT3 | rs2924488 | AGGGGAGGAATGTAGTCCCACTCTACAGTCAACACGGAGTGAGCCG | (SEQ ID NO: 159) |
| | | CCATGCATTGAAGAACACATGTCATCTCCAGGCCCAAGGTTCTTATT | |
| | | CACAACC[C/T]CTTTTTTTTTTTTTTTTTTTTTTTTTTTTTTTTTTTTTTTT | |
| | | GGAGATAGAGTTTCACTCTTGTTGCCAAAGCTAGAGTGCAAAGGCA | |
| | | CGATATTGGGTCA | |
| STAT3 | rs744166 | CATGCTCAGCATGGTAAATGTCATGGCAGGAGTGCCAACATTGAGA | (SEQ ID NO: 160) |
| | | GGGGAATTGGGCCACACAGTCTCTAAAAACTGTTTGTTCTATAAATT | |
| | | ACTGTCA[A/G]GCTCGATTCCCTCAAGACATTACAGCCACAGCAACT | |
| | | CAAAATAATACTGTAGGAAAAGCAAGTAGATATTTAACCAAAAAGG | |
| | | GTTCAGGGTTTTGTACTCC | |
| STAT3 | rs744284 | GGAGCCAAGAGGAGACTGATACGCCCCTGTGCTGGCTGTTCCGACA | (SEQ ID NO: 161) |
| | | GTTCGGTGCTCCTCCCTCCGCCACAGCGAGGGAAGAGCGGAGGACTT | |
| | | GGGCACA[A/G]AAGCCCGGGGGGAGGGAGGGAAGATGACCGGAATG | |
| | | TCCTGCTGAAAACTCAGCTGAGTTCCTGGCAGTGCGTGACGTCAAGG | |
| | | CACTTTAAATGCCCCTGA | |
| STAT3 | rs760282 | CTCCTGCCTCAACCTCCTGAATAGCTCCTGAATAGAATTACAGGCAC | (SEQ ID NO: 162) |
| | | AGGCCACCACAACCAGCTAATTTTTGTATTTTTAATAGAGACAGGGT | |
| | | TTCACC[A/G]TGTTAGCCAGGGTGGTCTCAAACTCCTGACCTCAAGTG | |
| | | ATCCACCCACCTCAGTCTCCAAAAGTGTTGGGATTACAGGCGTGGGC | |
| | | CACTGCACCAGGCCC | |
| STAT3 | rs957970 | CTCGAATTCCTGGGCTCATGTGATCTTCCTGCTTCAGCCTCCTGAGTA | (SEQ ID NO: 163) |
| | | GCTGGGACTAGACGTCCACTACTGCTCCTGGCTGGAAGTTTAGATTT | |
| | | TAATTTAAACTCTTCTATTGGGAAACTTTGTATGTTTGCTTTACCACT | |
| | | TAACATTTGCATGCATTTTATTGTACCTATTGTCTCCTACTTAAGGAAGG | |
| | | GCAGTTTATGCTGTTATATGAAGTGAATTAACCTCCTATCGTACTTCA | |
| | | GTTTTCTCTATGCTAAAAGTGTGTTC[C/T]AGATTTTTGAAAAACTTA | |
| | | CTTAATTTTCATTCATTTATTCAAATATTTGAGCATTCTGTAGTTGCT | |
| | | GGGGAAATAGCAGTGAACTGAAGAATGTCTTTGTTCTTATGGGGCTT | |
| | | AAGTTCCTAGTTGATCATATTGGAAGGAGATACATGAAAAAAGAAA | |
| | | TATATGAACAATGGAGGGCGATGAGTACTGTAAAGGAGAATTC | |
| STAT3 | rs957971 | CTCGAATTCCTGGGCTCATGTGATCTTCCTGCTTCAGCCTCCTGAGTA | (SEQ ID NO: 164) |
| | | GCTGGGACTAGACGTCCACTACTGCTCCTGGCTGGAAGTTTAGATTT | |
| | | TAATTTAAACTCTTCTATTGGGAAACTTTGTATGTTTGCTTTACCACT | |
| | | TAACATTTTGCATGCATTATTGTACCTATTGTCTCCTACTTAAGGAAGG | |
| | | GCAGTTTATGCTGTTATATGAAGTGAATTTAACCTCCTAT[C/G]GTACT | |
| | | TCAGTTTTCTCTATGCTAAAAGTGTGTTCTAGATTTTTGAAAAACTTA | |
| | | CTTAATTTTCATTCATTTATTCAAATATTTGAGCATTCTGTAGTTGCT | |
| | | GGGGAAATAGCAGTGAACTGAAGAATGTCTTTGTTCTTATGGGGCTT | |
| | | AAGTTCCTAGTTGATCATATTGGAAGGAGATACATGAAAAAAGAAA | |
| | | TATATGAACAATGGAGGGCGATGAGTACTGTAAAGGAGAATTC | |
| STAT5A | G3469a10 | AAACCCAGGTGACACCTGGGACGTGTTGAGTTCTAGTCCCTGGGAG | (SEQ ID NO: 165) |
| | | GAAATTAGATGGACCCTGTTTGGAATACTCTAGTGGAGGGCAGCTCT | |
| | | GACATAGATATTTTGGACTTTGAAGGTGTTACTATCTGCCGTTATCTG | |
| | | CCAGGTCACCTGTTTCTCCTTTCTCTTTCCCTTTTCTATCCCAATTCTG | |
| | | GCAAATGACTCCTTCTGATGCAAATGATCCT[T/C]CCTTCTTGCCCTA | |
| | | GTTTCCCTTCTTACCCTAGTTTGGGGTTGGGGTTTGGGGTCTGNAGT | |
| | | ATTGGTGTTTCCTAATGCCTGTGGTCTTCTCCCATCCTCTCTTGCCCG | |
| | | AGTTATTTCCATTCCATCTGTCTCCAGTGCAGGTCTCCGGCTGGGATC | |
| | | CTGGTTGACGCCATGTCCCAGAAGCACCTTCAGATCAACCAGACATT | |
| | | TGAGGAGCTGCGACTGGTCAC | |
| STAT5A | G3469a11 | TTAGATGGACCCTGTTTGGAATACTGTAGTGGAGGGCAGCTCTGACA | (SEQ ID NO: 166) |
| | | TAGATATTTGGACTTTGAAGGTGTTACTATCTGCCGTTATCTGCCAG | |
| | | GTCACCTGTTTGTCCTTTCTCTTTCCCTTTTCTATCCCAATTCTGGCAA | |
| | | ATGACTCCTTCTGATGCAAATGATCCTNCCTTCTTGCCCTAGTTTCCC | |
| | | TTCTTACCCTAGTTTGGGGTTTGGGGTTTGGGGTCTG[T/C]AGTATTG | |
| | | GTGTTTCCTAATGGCTGTGGTCTTCTCCCATCCTCTCTTCCCCGAGTT | |
| | | ATTTCCATTTCCATCTGTCTCCAGTGCAGCTCTCCGGCTGGGATCCTGG | |
| | | TTGACGGCATGTCGCAGAAGCACGTTCAGATCAACCAGACATTTTGAG | |
| | | GAGCTGCGACTGGTCACGCAGGACACAGAGAATGAGCTGAAGAAAC | |
| | | TGCAGCAGACTCAGGAGTAGT | |
| STAT5A | G3469a13 | CTGGTTCCTCAATCAGACTTTGGTCCCCATCCTGTGCACCTCCCCCAG | (SEQ ID NO: 167) |
| | | GAAGGGGGCTGCTGTCCTGGGGGTGGGATGGGGCTCGGGTGTGTGG | |
| | | GGTGATGCTTGGGCTGTTTGGGCCTAGTCAGGGTCGCCCCTCCTGTG | |
| | | TACGTCTCTAATTCTGGGAGGCAGGGAGGTCTGCTCTTCCCATGGGT | |
| | | GGGAAGTGTGGCGAAAGCACAGAGCGTTCCTGGGGGAACGGGAGCT | |
| | | GTGTCTTGGGG[C/A]CTGGCGTCTGTGAGGAGAAGCCATTGTCCTGCT | |
| | | GTTTGGCCTTGGGGCTCTCGTGCAGGTGTGAGAAGTTGGCCGAGATCA | |
| | | TCTGGCAGAACCGGCAGCAGATCCGCAGGGCTGAGCACCTGTGCCA | |
| | | GCAGCTGCCGATCCCCGGCCCAGTGGAGGAGATGCTGGCCGAGGTC | |
| | | AAGGCCACCATCACGGACATTATCTCAGCCCTGGTGACCAGGTGACT | |
| | | GCTGCCTGTTTGCCATGCCCAGGAGCTTGGG | |
| STAT5A | G3469a15 | AGACTGGGTGGGGGCAGGAGGGCCCTGACTTTCCTGGGCCACCTGT | (SEQ ID NO: 168) |
| | | GACCTGGGGCACCAGCCCTGACTCGGGGGTTCCTGGGCCCTCAGGA | |
| | | GAACTTGCCGGGCTGGAACTACACCTTCTGGCAGTGGTTTGACGGGG | |
| | | TGATGGAGGTGTTGAAGAAGCACCACAAGCCCCACTGGAATGATGG | |
| | | GTAAGGAACGGGGGCTGCAGGGTCAGGGGCCAGCTGTGGGCGCAGA | |
| | | G[A/G]GACTGTGGCTGTGGCCCAGTGGTGACGCTCAATGCTCCGTGC | |
| | | ACCCAGGGCCATCCTAGGTTTTGTGAATAAGCAACAGGCCCACGAC | |
| | | CTGCTCATCAACAAGCCCGACGGGAGCTTCTTGTTGCGCTTTAGTGA | |
| | | CTCAGAAATCGGGGGCATCACCATCGCCTGGAAGTTTGANTCCCGTG | |
| | | AGTGCCCGTTTTGCCCACACTCCAGCCCCAA | |
| STAT5A | G3469a15 | AGACTGGGTGGGGGCAGGAGGGCCCTGACTTTCCTGGGCCACCTGT | (SEQ ID NO: 169) |
| | | GACCTGGGGCACCAGCCCTGACTCGGGGGTTCCTGGGCCCTCAGGA | |
| | | GAACTTGCCGGGCTGGAACTACACCTTCTGGCAGTGGTTTGACGGGG | |
| | | TGATGGAGGTGTTGAAGAAGCACCACAAGCCCCACTGGAATGATGG | |
| | | GTAAGGAACGGGGGCTGCAGGGTCAGGGGCCAGCTGTGGGCGCAGA | |
| | | G[A/G]GACTGTGGCTGTGGCCCAGTGGTGACGCTCAATGCTCCGTGC | |
| | | ACCCAGGGCCATCCTAGGTTTTGTGAATAAGCAACAGGCCCACGAC | |
| | | CTGCTCATCAACAAGCCCGACGGGACCTTCTTGTTGCGCTTTAGTGA | |
| | | CTCAGAAATGGGGGGCATCACCATCGCCTGGAAGTTTGANTCCCGTG | |
| | | AGTGCCCGTTTTGCCCACACTCCAGCCCCAA | |
| STAT5A | G3469a17 | TGCAGGGTCAGGGGCCAGCTGTGGGCGCAGAGNGACTGTGGCTGTG | (SEQ ID NO: 170) |
| | | GCCCAGTGGTGACGCTCAATGCTCCGTGCACCGAGGGCGATCCTAGG | |
| | | TTTTGTGAATAAGCAACAGGCCCACGACCTGCTCATCAACAAGCCCG | |
| | | ACGGGACCTTCTTGTTGCGCTTAGTGACTCAGAAATCGGGGGCATC | |
| | | ACCATCGCCTGGAAGTTTGA[C/T]TCGCGTGAGTGCCCGTTTTGCCCA | |
| | | CACTCCAGCCCCAAGGCCCGGTCTCTTGTTCCCTTGCCCCGCCAGCC | |
| | | CACCCTCCATCGGGCCTGTGTCCTTAGAAGGTACCCAGCGGGAAGCT | |
| | | TAGTATGAGAGGGCTGTGGCTTGGAAATGTATTCTCTTTCTATTGTTT | |
| | | TCCATTTTTGGAGAACCTGAAGTCCCCAGCCCCATAGACTCCAGGACG | |
| | | GCTGGGCGAGTCCTCCTGCAGTTTC | |
| STAT5A | G3469a18 | TGGGTTTGCTTGTTGATTCTCATTCTTTGACAGGGGTGGGAGCAGGG | (SEQ ID NO: 171) |
| | | AGAGGGAAATCAGATGGCCAGAAAAAGAACCAGAAGGAATGGGAT | |
| | | TCAAGCCAGGGGTCTCAGTGACCCTCAGGGAGGATTCATCAGCTGGT | |
| | | GTTTATTGGGGGTCCTTGGGAAATCTCATCCCAGCTGAGAACACAAG | |
| | | GTGATGTGAGCAGGAGGGAGACT[A/G]CATGGGGCGTGGGNTTCCAC | |
| | | CCCACTTGGGAGTTCCCAGAGACTTTGGTTCTCACCACTGTTCTTCCC | |
| | | TTGNGAGCTAAAGCTGTTGATGGATATGTGAAACCACAGATCAAGC | |
| | | AAGTGGTGCCTGAGTAAGTGTCCAGGTGGGTGTGGCTGTGCTTCTGC | |
| | | CTCTTTCCTCCTCCCCCAGACCCTGCCTCCCATCCTGATCCTGGGCCC | |
| | | AGCTTTCCGCTCCCCCAGGGAACCTGT | |
| STAT5A | G3469a19 | CAGCAAAAGGGAGAAGTCTCTCTTCTTCCAGCTGCCCCAAATCCATT | (SEQ ID NO: 172) |
| | | GGTTGGTTTGGTTGTTGATTCTCATTTCTTTGACAGGGGTGGGAGCA | |
| | | GGGAGAGGGAAATCAGATGGCCAGAAAAAGAACCAGAAGGAATGG | |
| | | GATTCAAGCCAGGGGTCTCAGTGACCCTCAGGCAGGATTCATCAGCT | |
| | | GGTGTTTATTGGGGGTCCTTGGGAAATCTCATCCCAGCTGAGAACAC | |
| | | AAGGTGATGTGAGCAGGAGGGAGACTNCATGGGGCGTGGG[C/T]TTC | |
| | | CACCCCACTTGGGAGTTCCCAGAGACTTTGGTTCTCACCACTGTTCTT | |
| | | CCCTTGNCAGCTAAAGCTGTTGATGGATATGTGAAACCACAGATCAA | |
| | | GCAAGTGGTGCCTGAGTAAGTGTCCAGGTGGCTGTGGCTCTCCTTCT | |
| | | GCCTCTTTCCTCCTCCCCCAGACGCTGCCTCCCATCCTGATCCTGGGC | |
| | | CCAGCTTTCCGCTCCCCCAGGGAACCTGT | |
| STAT5A | G3469a4 | GCAGAACGGGCAGCAGATCCGCAGGGCTGAGCACCTCTGCCAGCAG | (SEQ ID NO: 173) |
| | | CTGCCCATCCGCGGCCGAGTGGAGGAGATGCTGGCCGAGGTCAACG | |
| | | CCACCATCACGGACATTATCTCAGCCCTGGTGACCAGCACATTCATC | |
| | | ATTGAGAAGCAGCCTCCTCAGGTCCTGAAGACCCAGACCAAGTTTGC | |
| | | AGCCACCGTACGCCTGGTGGTGGGCGGGAAGCTGAACGTGCACATG | |
| | | AATGCCCCCCAGGTGAAGGCCACCATCATCAGTGAGCAGCAGGCCA | |
| | | AGTCTCTGCTTAAAAATGAGAACACCC[G/A]CAACGAGTGCAGTGGT | |
| | | GAGATCCTGAACAACTGCTGCGTGATGGAGTACCACCAAGCCACGG | |
| | | GCACCCTCAGTGCCCACTTCAGGAACATGTCACTGAAGAGGATCAA | |
| | | GCGTGGTGACCGGCGGGGTGCAGAGTCCGTGACAGAGGAGAAGTTC | |
| | | ACAGTCCTGTTTGAGTCTCAGTTCAGTGTTGGCAGCAATGAGCTTGT | |
| | | GTTCCAGGTGAAGACTGTGTCCCTACCTGTGGTTGTCATCGTCCACG | |
| | | GCAGCCAGGACCACAATGCCACGGCTACTGTGCTGTGGGACA | |
| STAT5A | G3469a6 | GTCCCAGAAGCACCTTCAGATCAACCAGACATTTGAGGAGCTGCGA | (SEQ ID NO: 174) |
| | | CTGGTCACGCAGGACACAGAGAATGAGCTGAAGAAACTGCAGCAGA | |
| | | CTCAGGAGTACTTCATCATCCAGTACCAGGAGAGCCTGAGGATCCA | |
| | | AGCTCAGTTTGCCCAGCTGGCCCAGCTGAGCCCCCAGGAGCGTCTGA | |
| | | GCCGGGAGACGGCCCTCCAGCAGAAGCAGG[T/C]GTCTCTGGAGGCC | |
| | | TGGTTGCAGCGTGAGGCACAGACACTGGAGCAGTACCGCGTGGAGC | |
| | | TGGCCGAGAAGCACCAGAAGACCCTGCAGCTGCTGCGGAAGCAGGA | |
| | | GACCATCATCCTGGATGACGAGCTGATCCAGTGGAAGCGGCGGCAG | |
| | | CAGCTGGCCGGGAACGGCGGGCGCGCCGAGGGCAGCCTGGACGTGC | |
| | | TACAGTCCTGGTGTGAGAAGTTGGCCGAGATCA | |
| STAT5A | G3469a9 | CTCCTCAGAGGGTCCCTACCATCCAGGCCCTTTGGCCTCTAGTTTTAC | (SEQ ID NO: 175) |
| | | TCATGGTGGTTTGGTGTGACTCTGGTCCTGCCTGTCCTTCTTTGTGCG | |
| | | GAAGGGGTGGTGCCCCTGGGAAAGGGGAAGCCTGGGAGGACAGAG | |
| | | AATGTTTTTAGACTCGGATGTCTGTGGAGCTGCTGGGAAGAAGTCTT | |
| | | GTGGGCCCTGGAGTGCCCTCGGTCATGAGCCTGGGGTTTCCACTTTA | |
| | | TTCC[A/G]GCTCCCTGACCTCCTTGCCCAAGGAGGTGCCACAGTAGGT | |
| | | TTTTCTCTTCTGCCCTGCCCCAAGGGATGCCATTGACTTGGACAATCC | |
| | | CCAGGACAGAGCCCAAGCCACCCAGCTCCTGGAGGGCCTGGTGCAG | |
| | | GAGCTGCAGAAGAAGGCGGAGCACGAGGTGGGGGAAGATGGGTTTT | |
| | | TACTGAAGATCAAGCTGGGGGACTAC | |
| STAT5A | rs1057935 | CTGAATTAGTCCTTGCTTGGCTGCTTGGCCTTGGGCTTCATTTCAAGTC | (SEQ ID NO: 176) |
| | | TA[C/T]GATGCTGTTGCCCACGTTTCCCGGGATATATATTCTCTCCCCT | |
| | | CCGTTGG | |
| STAT5A | rs1840166 | GGAGGTCGAGACCAGCCTGGCCCGCTTGGTGAGACCCTGTCTCTACT | (SEQ ID NO: 177) |
| | | AAAAATACAAAAATTAGCCGGGCGTGGTGGCACATGTGTGTAGTCT | |
| | | CAGGAGG[A/C]TGAGGCAGGAGAATCATTTGAACACAGGTGGCAGA | |
| | | TGTTGGAGTGAGCTGAGATTGCACCACTGCACTTCAGCCTCGGCGAC | |
| | | AAAATGAGACTCTGTCTC | |
| STAT5A | rs2002052 | CCAGCCGCCCACCAGGGCCCACCGGGGTCTGTTGCTCCTCCACCTCA | (SEQ ID NO: 178) |
| | | CAGACAGGTTACTGGCTGGGGCTAGCACCCCACTCTCCACCCCCAAC | |
| | | CACTCTCTCCTAGAACCAGGAGAGATGGGGGCCCCTAGCCTGAAGA | |
| | | AATGCAGGCAGGGGCTATAGGAGTTGTGCTTAGTTGTGAAGCCAAG | |
| | | AGCGAGATGACGGAGGGCCCAGGGCTGGGAGTCAGGATCCTTGGGT | |
| | | TCTGGGGCTAGATGTGTTTGCTAATTTTCTTTTTTTTTTTTAGAGACA | |
| | | GAATCTCATTTCTGTCACCCAGGCTGGAGTGCAGTGGCGTGATTTCAG | |
| | | CTTACTGTAACCTCTGCCTCCCGGGTTCAAGTGATTCTCGTGCCTCAG | |
| | | CCTCCTGAGTAGCTGGAATTATAGGTGCCTGCCACCATGCTGGGGTA | |
| | | ATTTTTGTATTTTTAGTAGAGATGGGGTTTCACCATGTTGGTCAGGCT | |
| | | GGTCT[C/T]GAACTCCTGACCTCAGGTGATCCGCCCACCTCGGCCTCC | |
| | | CAAAGTGCTGGGATTACAGGCGTGAGCCACCACGCCTGTGGCTCTG | |
| | | GCTCTCCTTCTGCGTCTCTCCTTGGTAATTTTTGAAGACCCATCCATT | |
| | | CTCTGTGCCTCAGTTTCCCACTGTAAAATGAGGGGATGACTTTGGAG | |
| | | GAAATGACTTGGAGGCCCCATCCAGCCTAGATTATCTTTACACCTCT | |
| | | TACTTCCACCTTGGGCTGGTCTTGGACCCCTGTGTCTTAGAAGTGAG | |
| | | TTCCCTCTGTGTTTTCTAGGGACCAAGTTGGGGCTGTGTAATTTATGT | |
| | | CTGCTGGATGTGGGGGCAGCAAGGTGAAAAACTGGGAAAGACTGGT | |
| | | GGG | |
| STAT5A | rs2049238 | AAGAAATGGCTAGAACTTCTGTGTTTCTTTGGATATTGCCTGACATC | (SEQ ID NO: 179) |
| | | TAAGCCAAATTAGTTGACCTTTTAATGTTAAATAAAATATTCAATAA | |
| | | CTTTTA[A/C]TAATGGGTGGGCTTTTAGGAACCCCTTTATTTTACTATG | |
| | | TTGCCAAAAGTAGGTTCATTATAACATTGGAAACATCCTGATGTAAT | |
| | | TTATTGAATGTAAAA | |
| STAT5A | rs2883375 | TTGTTTATTTATTTTTGAGACTGAGTCTCACTCTATCATCTAGGCTGG | (SEQ ID NO: 180) |
| | | AGTGCAGTGGCGTGATCTCGGCTCACTGCAACCTCCACCCCTTGGGT | |
| | | TCAAG[A/C]GATTCTCATGCCTCAGCCTCCCAAGTAGCTAGGATTACA | |
| | | GGGGCCCACCACCATGCCCGGGTAATTTTTGTATTTTTAGTAGAGAC | |
| | | AGGGTTTCACCCTG | |
| STAT5A | rs2948176 | CCACCATCACGGACATTATCTGAGCCCTGGTGACCAGGTGACTGCTG | (SEQ ID NO: 181) |
| | | CCTGTTTGCCATGCCCAGGAGCTTGGGGCAGCTCCTGCCTGCGTGGG | |
| | | GGGAGC[C/T]GCAGGTGCCTTCCAGACCAGCAGATCGACTTCGTGCC | |
| | | TCTCATCCCTCCCAACTCCATCTCCAGTTGCTGTGGCCCTTGCCCAGT | |
| | | TTCTCCTGTGGACAC | |
| STAT5A | rs2948177 | CAATGCTAGCTTTTTAGGAGCTAGGACTACCCACACTAAAGCCAAGC | (SEQ ID NO: 182) |
| | | CCGGCCCAACCAAGACAGATGTTTGGTGGGTGGGGCTGCCTGCTGG | |
| | | AGCAACA[G/T]GCCTCTGCAGGCCCTGGAGGAGAAGTCTAGAGAGGT | |
| | | GGGTGCGTGGAAGAGAGCAGGCAGGACCCCCTCTCTTAAGCAGGCA | |
| | | CGCGGACCCCATGAGGTG | |
| STAT5A | rs909056 | AAGCGAGGGAGAGGGGCGGCGCGTGGGCCCCAAGAGGGAGGTGCA | (SEQ ID NO: 183) |
| | | GGGGGCCCAGAGGAGGGGGAAGGCGGGGGTGGGAGAAGAGAGGAG | |
| | | GGGAGGGGAC[C/G]GGCAGGTGCCACCGCCCCAGGGGGCTAATCTG | |
| | | ATCCATCTGTGCGAGGGAAGGTGCTCGCTCGTAGCTCCCAGGCGCGG | |
| | | TCTCCAGGGGGCTGGGGCACG | |
| STAT5A | rs986254 | TTTTTAAAATTGTATTCACTGTTAATTATAGTGGGCTTATTTGAAGTA | (SEQ ID NO: 184) |
| | | AAATCTTGACATTGGGTGTTTTATTAATGATGATCCTCTGCTAGTGAA | |
| | | AAGC[C/T]TATGTCAGTTTGGAGAAGATTTTGCACATCTAAATCTATG | |
| | | CTGTTTTTACTAATCAAAAGAGAAGAGCAATTTTAGAGGTGTTAGATT | |
| | | TTTAGTTGGTTGA | |
| STAT5A | rs986256 | CTTTTATTTTGATACTATAAAATATCAAAGTATCAAAATACTTTAACT | (SEQ ID NO: 185) |
| | | GTGCAACTTAATAAAAATAATGCTTGAAAGTTAGCTCTGCACTATCG | |
| | | CTGTC[C/T]TCAAATGTGTGGTATTTAAAGGAAGAAAATGTTTTTAAT | |
| | | TCTGAGCTTTGTGAATAGAACTCTTCTAAAATCAGAAGTTTTTTTTTT | |
| | | TCTCCTGCAGTGT | |
| STAT5A | rs994607 | AAATCATCTATGGTGACTTCGAGCTGTGGCACTTGGCTTTCATTCC | (SEQ ID NO: 186) |
| | | AGTTGACCCCCTAGCTCTGTGTCTGACCCTCCCCTGCCAAATCCATTG | |
| | | CTCA[G/T]AGTGGGAAAGGAGAGGAGAGGGACTATACTTCCTCCTCC | |
| | | CTGGGGCCCCCTGCAGAGCATCTGGGAAGCAAGGCTTCCCTACATCC | |
| | | TCCATGCACGCCCT | |
| STAT6 | rs167769 | CCCAAACCCAGTAACCCCAAGTCCTCCTGTTGCACGTTCAGCCTCTC | (SEQ ID NO: 187) |
| | | ACCTCTCCCAGGCCTCTCAGAGGGATGGAAAATGGAGAGGTCCCTTC | |
| | | TCTGGA[C/T]GTTTCTGCTCCAAGACCTTCATGCCCCTCTTTCCTCCAG | |
| | | TGCCCACTTACCCCAGCTCTTTTCTCCTCAAGGCAGAATCCTTACCCC | |
| | | TCCTGCTCAGGTT | |
| STAT6 | rs2598483 | GCAGGCAGGGACTCTGTCCATTGGCTTGTCCAACAAGCCATGCTAGG | (SEQ ID NO: 188) |
| | | ATCAAGGTGTGTGCACATACGTGTTCACGGCACACGTGCTTTGTATG | |
| | | TGCTCC[C/T]GAGTGCCCACTCTGCCTGCAGACATCCACAGACAGTA | |
| | | GGTGAAGCCAGGTTTCACCATCTAAGGGTGTCAGAGCTACAAGTCCC | |
| | | CAAAGACTTGGAGAGG | |
| STAT6 | rs2626577 | TCAGATAAGGTTGAGATCCAAGAGGCAACCACTCCTACACACTCCCC | (SEQ ID NO: 189) |
| | | AGAACCACCCTGGCCCCCACCTTGGCCAGCCTCAGCCCCCTTCTGCA | |
| | | GGGCTT[C/G]TCGGAGAAGGTGGATGTCCCCTACTCGGTGCTGCAGC | |
| | | CTCCGCAAGCCTGTCTTAAACTTGAGTTCTTCCTGCTTCCAGTGGAA | |
| | | AGGCATTGGCAAGTGG | |
| STAT6 | rs2629440 | CTGTCTTTCTCCCTTTCTCAGCGGGCACTTCCAGGCCCCATAGGTCTGT | (SEQ ID NO: 190) |
| | | AGCTCTGTCCAGCGAGTTCAAGGCTGGCCCTGCTAGCACCTCCCCCC | |
| | | TTCCA[A/C]CACCACCACCAAAAAGAAAAATAAGATAAAGCACACA | |
| | | CATACATACACACACACACACACACACACACACACACTCCTCTCCCG | |
| | | TCCTCCCTCTTCAGTG | |
| STAT6 | rs3001428 | GCTGAGGGGCCAAAGCTAGGCCTATGCTGCCCCCTGGTGCCTGAATT | (SEQ ID NO: 191) |
| | | GATTTCCAGAACCAGCTCCTCCTCCTCCATCCCTTGCTACCCCGCAG | |
| | | GTCTGT[A/G]CATGAGTGCACGTGTGTGGACCCAGCCAGGCAGCAGA | |
| | | GGGTTTGAACACAGCAAAATATAGCAGTTAAAAGACTGGACTGTGG | |
| | | ATACAGGCTGCTTGGGT | |
| STAT6 | rs3024954 | AAGGGGAGTTGGGGGCTAGGTCCCTGCTGGTGCCCTCCCCACAGCTC | (SEQ ID NO: 192) |
| | | TAGCCCCCTTCTTTTCTTCCCGAGGTCTGTTCTAGGCCAGACTGGGAG | |
| | | CTCCC[A/G]GGGAATACCTGGTGACGAGGGTTCTCAGGACTTCATCC | |
| | | AGCCGGCCAGTCAGCGATGCCCGGGTCTTGGGCTCAAGCTCCCCACC | |
| | | AGCCGCCCCTACCTC | |
| STAT6 | rs324011 | CCTGGAGGAGAAAAATAAGGCCACTCTGAGGGGTGCCCAAGAAACT | (SEQ ID NO: 193) |
| | | TGGCCTATCTCCTGGGGCAGCCAGGGACCTCCCATAGATAGCCCTCC | |
| | | TAGGGAC[C/T]GTCCCCACCACCACTCATGGCCAGACCACCTTAAAC | |
| | | CAGGGGCATCCTGAGTCAATGCCTGAGATGGGGGTAACTCCTTCAGT | |
| | | GATAGACACAGGGGTGG | |
| STAT6 | rs324012 | AGGCTGAGGTGACAGAATGGCGTGAACCTGGGGGGCGGAGCTTGCA | (SEQ ID NO: 194) |
| | | GTGAGCAGAGATCGAGCCACTGCACTCCAGCCTGGGCGACAGAGAG | |
| | | AGACTCTG[C/T]CTCAAAATAAATAAATAAATAAATGAAATAAAATA | |
| | | AAAATGAAAACCACAAGGCTAGATGATGAAGAGGATGGGAGAGTA | |
| | | ATAACAGCTACTATTTGTTA | |
| STAT6 | rs324013 | AGGGATCAGCCTAGCTGCAATGTGGAGGATGGATTTAAAAGATAAG | (SEQ ID NO: 195) |
| | | TTCTGAAGCCAGGAGATCCATTCAGAAGGTTAATAACAACAGAGAG | |
| | | GGAGACGA[C/T]ACAGTTGAAGAGCAGAGGTGAAGCATTGTGGAAT | |
| | | GGAGACTGGTTAGGAAGAAAAGTGAAGACATGAGGGGCCAGTGTCA | |
| | | AAGGAAAGAAAGGCAAGAAA | |
| STAT6 | rs841718 | TCTGGGGTTAGGGAGGAAGGGAGGTGGAAAAGGTGGGCATGGATCA | (SEQ ID NO: 196) |
| | | TGGGGAAGTAAGAGAAGCACAGCTATGAAATAGGGAGTGACATCAG | |
| | | GATGACAC[A/G]CGGGCAGGGAGAGGAGGGCAGCGGGGAGCAGGG | |
| | | AGGAAGTGGGTGACAGGAAGGAATCAGAGCTGCCAGTTCCAGCTCA | |
| | | CGCTTGTAGTGGCTCCGGAAA | |
| TBX21 | TBET_3′UTR001 | AGGACAGTTTTTATAACTATTTTCCCAACTGAGCAGATGACATGATGA | (SEQ ID NO: 197) |
| | | AAGGAACAGAAACAGTGTTATTAGGTTGGAGGACACCGACTAATTT | |
| | | GGG[A/T]AACGGATGAAGGACTGAGAAGGCCCCCGCTCCCTCTGGCC | |
| | | CTTGTCTGTTTAGTAGTTGGTTGGGGAAGTGGGGCTCAAGAAG | |
| TBX21 | TBET_Ile339Val | AGGGTGGGGGACAGATGCTACAGGTGGGCAGGCCAGGGAAGGAGG | (SEQ ID NO: 198) |
| | | GTCGGAGAAGGAATGTGTGAAACAGGTAGGCTCACAGGTGACTGGT | |
| | | TCTGCTTGTGACCCGTTTTNTTGCCTTCTATTTTTTTCTAGCATGTACA | |
| | | CATCTGTTGACACCAGC[A/G]TCCCCTCCCCNCCTGGACCCAACTGTC | |
| | | AATTCCTTGGGGGAGATCACTACTCTCCTCTCCTACCCAACCAGTAT | |
| | | CCTGTTCCCAGCCGCTTCTACCCCGACCTTCCTGGCCAGGCGAAGGA | |
| | | TGTGGTTCCCCAGGCTTACTG | |
| TBX21 | TBET_Pro485Pro | CTGGTTCCGCCCTATGCGGACTCTGCCCATGGAACCCGGCCCTGGAG | (SEQ ID NO: 199) |
| | | GCTCAGAGGGACGGGGACCAGAGGACCAGGGTCCCCCCTTGGTGTG | |
| | | GACTGAGATTGCCCCCATCCGGCC[G/A]GAATCCAGTGATTCAGGAC | |
| | | TGGGCGAAGGAGACTCTAAGAGGAGGCGCGTGTCCCCCTATCCTTCC | |
| | | AGTGGTGACAGCTCCTCCCCTGCTGGGGCCCCTTCTCCTTTTGATAA | |
| | | GGAAGCTGA | |
| TBX21 | TBET_rs1989291prom | GGGATTACAGGGGCCAGCCACCACACCTGCTAATTTTTATTTTTGTTT | (SEQ ID NO: 200) |
| | | TTGTTTTTGTTTTTGAGATGGAGTCTTGCTCTGTCTCCCGGGCTGGAGT | |
| | | GCAG[C/T]GGCACGATCTCGGCTCACTGCAACCTCTGCCTCCTGGGTT | |
| | | CAAGTGATTCTCCTGCCTCAGCCTCCTGAGTAGCTGGGATTACAGGC | |
| | | GCCCATCATGCCC | |
| TBX21 | TBET_rs2074190 | CGGACGCCGAGGGCTACCAGCCGGGCGAGGGCTACGCCGCCCCGGA | (SEQ ID NO: 201) |
| | | CCCGCGCGCCGGGCTCTACCCGGGGCCGCGTGAGGACTACGCGCTA | |
| | | CCCGCGGG[A/G]CTGGAGGTGTCGGGGAAACTGAGGGTCGCGCTCAA | |
| | | CAACCACCTGTTGTGGTCCAAGTTTAATCAGCACCAGACAGAGATGA | |
| | | TCATCACCAAGCAGGGAC | |
| TBX21 | TBET_rs2158079 | TTGTGTTAGAAGAGAAAGGCTAAGCCATTGGAGCTGGTGATGGAAT | (SEQ ID NO: 202) |
| | | TGGTGCTGGTGGAGGTGGTGGTTGTAGTGACGGTACTACTGGTGGTG | |
| | | GTTGTGA[C/T]GGTGAAGGTGGTGGCTGTAGATGGTGGTGCCCGTGC | |
| | | TGGTGCTGGTGGAGATCGTGGTTATCGTGATGGTGGAGGGGGCAGTT | |
| | | GTAGTGACGGTGGTTGT | |
| TBX21 | TBET_rs2188895prom | CCCTCAACCTTCACATGACAGACCCAGTCAGTCCTGGCAGAGGTCTG | (SEQ ID NO: 203) |
| | | ATAGCTCCATTTACTGACAAAAAAAACCCAGAGAGGTTAAGATACT | |
| | | TTAATAG[A/G]TAGCACAGCCAGTAAGGGTTGGGATTGAGGTTCCAA | |
| | | CCCAGCCACCCAACTAAGTCTCCCCACCCCATTTCTCTCTGCCCCAG | |
| | | CTCCAGCACCCCCAGGC | |
| TBX21 | TBET_rs2240017 | GGATGGGCATCGTGGAGCCGGGTTGCGGAGACATGCTGACGGGCAC | (SEQ ID NO: 204) |
| | | CGAGCCGATGCCGGGGAGCGACGAGGGCCGGGCGCCTGGCGCCGAC | |
| | | CCGCAGCA[C/G]CGCTACTTCTACCCGGAGCCGGGCGCGCAGGACGC | |
| | | GGACGAGCGTCGCGGGGGCGGCAGCCTGGGGTCTCCCTACCCGGGG | |
| | | GGCGCCTTGGTGCCCGCCC | |

Results of the single allele analysis (subsetted to include only those with at least a marginally significant p-value) of the bronchodilator and steroid response phenotypes, respectively are shown below in Tables 3-6. Response 1 is a quartile response of high and low acute bronchodilator response, response 2 is an extreme response to bronchodilator (<15% change in FEV₁ from baseline vs >50% change in FEV₁ from baseline). Response 3 and 4 are steroid phenotypes. Response 3 is a quartile response for the change over 8 weeks of FEV₁ and response 4 is an extreme response to steroids (<−10% from baseline vs >+10% from baseline).

TABLE 3
Bronchodilator Response in the Forest
Group-Single Allele Analysis

| ANOVA | Gene | SNP | P-value |
|---|---|---|---|
| | egr1 | 1 | 0.02099 |
| | egr1 | 1 | 0.0408 |
| | MAPK8 | G1096a3 | 0.02509 |
| | STAT3 | G3363a16 | 0.00448 |
| | GATA3 | G9779a7 | 0.03939 |
| | FCER2 | G9782a0 | 0.05501 |
| | POMC | rs1009388 | 0.04556 |
| | STAT3 | rs1026916 | 0.01261 |
| | STAT6 | rs167769 | 0.03712 |
| | STAT3 | rs744166 | 0.01246 |
| | CRHBP | rs247742 | 0.01927 |
| | STAT3 | rs957971 | 0.03246 |
| | CRH | rs1870392 | 0.03486 |
| | IL18BP | rs949323 | 0.04294 |
| | CRHR1 | rs242949 | 0.04529 |
| χ² | STAT3 | rs1026916 | 0.00228 |
| | IL18BP | rs1573503 | 0.00402 |
| | IL18BP | rs1541304 | 0.00512 |
| | STAT3 | G3363a16 | 0.00826 |
| | IL18BP | G9772a3 | 0.02587 |
| | STAT5A | G3469a9 | 0.04631 |
| R1 | STAT6 | rs167769 | 0.04317 |
| | CRHR1 | rs242939 | 0.04235 |
| | STAT6 | rs324012 | 0.05972 |
| R2 | STAT3 | rs744166 | 0.05199 |
| | HSD11B1 | rs846911 | 0.0214 |
| | IL18BP | rs949323 | 0.01538 |
| | IL18BP | rs949323 | 0.00046 |
| | STAT3 | rs957971 | 0.0595 |
| | GATA3 | rs9746 | 0.01563 |

TABLE 4
Steroid Response in the Forest Group-Single Allele Analysis

| ANOVA | Gene | SNP | P-value |
|---|---|---|---|
| | IL18BP | rs2155145 | 0.00309 |
| | CRHBP | rs2135078 | 0.01581 |
| | CRHR1 | rs242941 | 0.01852 |
| | FCER2 | rs1990975 | 0.02276 |
| | TBET | INTRON4-22 | 0.02797 |
| | STAT6 | rs167769 | 0.02833 |
| | NR3C1 | rs6191 | 0.02994 |
| | STAT6 | rs324011 | 0.03339 |
| | FCER2 | rs889182 | 0.04559 |
| | STAT6 | rs324012 | 0.058 |
| | TBET | rs2074190 | 0.05886 |
| χ² | | | |
| R3 | Eotaxin | G195a2_CL | 0.04317 |
| | CRHR1 | rs242941 | 0.06648 |
| | FCER2 | rs1990975 | 0.07171 |
| | FCER2 | G9782a17 | 0.07585 |
| | FCER2 | G9782a0 | 0.09102 |
| | GATA3 | rs520507 | 0.09611 |
| | FCER2 | G9782a26 | 0.10015 |
| | CRHR1 | rs1876829 | 0.10619 |
| | CRHBP | rs2135078 | 0.10725 |
| | CRHR1 | rs1876827 | 0.10779 |
| | FCER2 | G9782a22 | 0.10937 |
| R4 | POMC | rs2071345 | 0.01829 |
| | TBET | INTRON4-22 | 0.02039 |
| | Eotaxin | G195a2_CL | 0.02375 |
| | TBET | rs2074190 | 0.03439 |
| | ALOX15 | rs2255888 | 0.03848 |
| | NR3C1 | rs852977 | 0.04517 |
| | NR3C1 | rs860457 | 0.05106 |
| | POMC | rs1866146 | 0.05406 |
| | TBET | rs2240017 | 0.05775 |
| | FCER2 | rs1990975 | 0.06584 |
| | NR3C1 | rs33389 | 0.07344 |
| | STAT6 | rs841718 | 0.07869 |
| | NR3C1 | rs6188 | 0.07927 |
| | CRHR1 | rs81189 | 0.08352 |
| | FCER2 | rs889182 | 0.08352 |
| | GATA3 | G9779a6 | 0.08956 |
| | CRHR1 | rs242950 | 0.09285 |

TABLE 5
Bronchodilator Response in the CAMP
Group-Single Allele Analysis

| ANOVA | SNP | Gene | P-value |
|---|---|---|---|
| | RS1042428 | FCER2 | 0.00069 |
| | RS242939 | CRHR1 | 0.07419 |
| | RS242950 | CRHR1 | 0.08499 |
| χ² | RS6196 | NR3C1 | 0.02341 |
| | RS852978 | NR3C1 | 0.06229 |
| | RS2240017 | TBET | 0.06331 |
| | RS1396862 | CRHR1 | 0.06375 |
| | G9782A19 | FCER2 | 0.07401 |
| | RS242949 | CRHR1 | 0.08284 |

TABLE 6
Steroid Response in the CAMP Group-Single Allele Analysis

| ANOVA | Gene | SNP | P-value |
|---|---|---|---|
| | CRHR1 | RS242939 | 0.00821 |
| | CRHR1 | RS242950 | 0.0601 |
| | NR3C1 | RS860457 | 0.065 |
| | NR3C1 | RS852977 | 0.08173 |
| | NR3C1 | RS6188 | 0.08814 |
| | FCER2 | RS1042428 | 0.09682 |
| χ² | CRHR1 | RS242939 | 0.00419 |
| | CRHR1 | RS242950 | 0.00992 |
| | FCER2 | G9782A8 | 0.04239 |
| | FCER2 | RS1990975 | 0.06123 |
| | STAT3 | RS744166 | 0.07008 |
| | CRHR1 | RS242938 | 0.07423 |

An average of 8 single nucleotide polymorphisms (SNPs) per candidate gene in the Forest population were genotyped, and single alleleic χ2 tests and analyses of variance (ANOVA) (Table 7) were performed for our primary outcome—change in FEV₁ over the trial period. For χ2 tests (Table 8), the outcomes were divided into quartiles and “extremes” that is responders with a more negative response than −10% vs. those who responded more than +10%.

TABLE 7
ANOVA for Steroid Response in the Forest Group

| Gene | SNP | P-value |
|---|---|---|
| IL18BP | rs2155145 | 0.00309 |
| CRHR1 | rs242941 | 0.01852 |
| FCER2 | rs1990975 | 0.02276 |
| NR3C1 | rs6191 | 0.02994 |
| FCER2 | rs889182 | 0.04559 |

TABLE 8
ANOVA for Bronchodilator Response in the Forest Group

| Gene | SNP | P-value |
|---|---|---|
| STAT3 | G3363a16 | 0.00448 |
| STAT3 | rs744166 | 0.01246 |
| STAT3 | rs1026916 | 0.01261 |
| STAT3 | rs957971 | 0.03246 |
| IL18BP | rs949323 | 0.04294 |
| CRHR1 | rs242949 | 0.04529 |
| FCER2 | G9782a0 | 0.05501 |

Preliminary haplotype analysis of the Forest and CAMP groups were performed using CLUMP (30). Table 9 demonstrates the “global” effect of the gene on the response phenotypes. Table 10 demonstrates that within some genes risk haplotypes have been identified. As each gene has multiple haplotypes, it is possible that a single haplotype will be a powerful indicator of a treatment response but be uncommon enough that the entire gene effect is essentially negative. CRHR1 is an example of this. The “column” number in the worksheet refers to a numbered haplotype in the clump input.

TABLE 9
Global Effects of the Genes on the Response Phenotype (Response 1-4)

| | Respond 1 | | Respond 2 | | Respond 3 | | Respond 4 | |
|---|---|---|---|---|---|---|---|---|
| | Chi Square | P- value | Chi Square | P- value | Chi Square | P- value | Chi Square | P- value |
| Alox15 | 0.09 | 0.96 | 1.48 | 0.48 | 2 | 0.37 | 4.34 | 0.11 |
| CRHBP | 4.76 | 0.2 | 19.4 | 0.001 | 2.08 | 0.71 | 3.1 | 0.54 |
| CRHR1 | 1.09 | 0.78 | 0.22 | 0.97 | 5.93 | 0.12 | 4.92 | 0.11 |
| Eotaxin | 0.71 | 0.87 | 4.33 | 0.23 | 1.4 | 0.7 | 5.95 | 0.11 |
| FCER2 | | | 23.7 | 0.005 | 15.9 | 0.02 | 24.9 | 0.005 |
| GATA3 | 3.35 | 0.34 | 2.32 | 0.51 | 2.49 | 0.37 | 4.61 | 0.2 |
| HSD11B1 | 1.33 | 0.86 | 2.96 | 0.39 | 0.3 | 0.86 | | |
| IL18BP | 10.2 | 0.01 | 8.68 | 0.03 | 18.2 | 0.001 | 0.04 | 0.97 |
| MAPK8 | 0.07 | 0.96 | 2.66 | 0.26 | 1.15 | 0.56 | 2.25 | 0.32 |
| NFATC4 | 1.07 | 0.9 | | | 0.67 | 0.95 | 1.81 | 0.76 |
| NR3C1 | 3.09 | 0.37 | 4.5 | 0.21 | 0.83 | 0.84 | 5.36 | 0.15 |
| POMC | 3.7 | 0.29 | 4.32 | 0.22 | 2.65 | 0.45 | 6.41 | 0.09 |
| STAT3 | 51.4 | 0.001 | 47.4 | 0.001 | 61.7 | 0.001 | 22.3 | 0.001 |
| STAT5a | | | 15.6 | 0.002 | | | | |
| STAT6 | 5.74 | 0.22 | 2.12 | 0.83 | 2.31 | 0.88 | 2.5 | 0.87 |
| TBET | 6.81 | 0.08 | 0.95 | 0.62 | 4.4 | 0.11 | 13.5 | 0.003 |
| | BD848 | | Achr8 | | Preppch | | P848 | |
| CAMP | Chi Square | P- value | Chi Square | P- value | Chi Square | P- value | Chi Square | P- value |
| CRHR1 | 1.13 | 0.77 | 10.7 | 0.01 | 1.05 | 0.79 | 12.2 | 0.006 |
| FCER2 | 5.6 | 0.68 | 11.8 | 0.15 | 14.1 | 0.08 | 20.4 | 0.04 |
| IL18BP | 10 | 0.6 | 2.19 | 0.33 | 15.4 | 0.001 | 1.73 | 0.42 |
| NR3C1 | 9.82 | 0.02 | 7.15 | 0.07 | 0.95 | 0.81 | 4.19 | 0.24 |
| STAT3 | 8.18 | 0.14 | 0.53 | 0.76 | 5.46 | 0.009 | 4.17 | 0.38 |
| TBET | | | | | | | | |

bold = low imputed frequency cell

TABLE 10
Genes with Identified Risk Haplotypes

| | Single Column Positivity | | | | | | | |
|---|---|---|---|---|---|---|---|---|
| | Respond 1 | | Respond 2 | | Respond 3 | | Respond 4 | |
| Gene | Column | P- value | Column | P- value | Column | P- value | Column | P- value |
| Alox15 | | | | | | | 1 | 0.03 |
| CRHBP | 4 | 0.03 | 4 | 0.01 | | | | |
| CRHR1 | | | | | 2 | 0.05 | 2 | 0.03 |
| Eotaxin | | | 3 | 0.06 | | | 2 | 0.03 |
| FCER2 | | | 7 | 0.003 | 7 | 0.01 | | |
| GATA3 | | | | | | | | |
| HSD11B1 | | | | | | | | |
| IL18BP | 4 | 0.006 | | | 3 | 0.00002 | | |
| MAPK8 | | | | | | | | |
| NFATC4 | | | | | | | | |
| NR3C1 | | | | | | | 4 | 0.06 |
| POMC | | | 3 | 0.07 | | | 2 | 0.02 |
| STAT3 | 1 | 0.00005 | 1 | 0.00002 | 6 | 0.00001 | 6 | 0.0009 |
| STAT5a | | | 2 | 0.002 | | | | |
| STAT6 | 4 | 0.07 | | | | | | |
| TBET | 3 | 0.04 | | | 1 | 0.04 | 2 | 0.0003 |
| | BD848 | | Achr8 | | Preppch | | P848 | |
| CAMP | Chi Square | P- value | Chi Square | P- value | Chi Square | P- value | Chi Square | P- value |
| CRHR1 | | | 1 | 0.01 | | | 3 | 0.02 |
| FCER2 | | | 6 | 0.03 | 1 | 0.02 | | |
| IL18BP | | | | | 2 | 0.001 | | |
| NR3C1 | 1 | 0.004 | 3 | 0.06 | | | 1 | 0.06 |
| STAT3 | 4 | 0.02 | | | | | | |
| TBET | | | | | | | | |

Haplotypes for each candidate gene were also inferred utilizing Phase (29). Clump (30) was utilized to screen for possible haplotypic effects of each of the candidate genes, using dichotomous outcomes. Secondary analyses were also performed utilizing the acute bronchodilator response at enrollment (all subjects were on steroids at the time of enrollment) as an endpoint (Tables 12 and 14). Final haplotypic analysis was performed utilizing the program haplo.score (31), which allows for analysis of continuous outcomes and for covariate adjustment of the haplotypic effects.

Using the Clump analysis, several imputed “risk haplotypes” were identified for the initial genes in Forest. The haplotypes for two genes, CRHR1 and IL18BP are shown below (Tables 11 and 12). The haplotypes for FCER2 are shown in Table 13.

TABLE 11
CRHR1-Clump Identified Risk Haplotype (Steroid Quartile Response)

| | | | | | | |
|---|---|---|---|---|---|---|
| Haplotype | # High | % | # Low | % | OR | CI |

| | | | | | | | |
|---|---|---|---|---|---|---|---|
| GCTCTTGTTCATCAGAG | (SEQ ID NO: 205) | 10 | 4.42 | 13 | 5.91 | 0.74 | (0.29-1.84) |
| GCCCCCATGCGGTGAGA | (SEQ ID NO: 206) | 34 | 15.04 | 51 | 23.18 | 0.59 | (0.35-.097) |
| GCCCCCATTCGTCAGAG | (SEQ ID NO: 207) | 65 | 28.76 | 50 | 22.73 | 1.37 | (0.88-2.15) |
| CTCACCACGAGTCAGAG | (SEQ ID NO: 208) | 86 | 38.05 | 88 | 40.00 | 0.92 | (0.62-1.37) |

TABLE 12

IL18BP-Clump Identified Risk Haplotypes
(Bronchodilator Response)

| | | | | | | |
|---|---|---|---|---|---|---|
| Haplotype | # High | % | # Low | % | OR | CI |

| | | | | | | | |
|---|---|---|---|---|---|---|---|
| ACCGGT | (SEQ ID NO: 209) | 160 | 50.63 | 44 | 39.29 | 1.59 | (1.00-2.52) |
| AGCGGT | (SEQ ID NO: 210) | 131 | 41.46 | 52 | 46.43 | 0.82 | (0.52-1.29) |
| GGAAAC | (SEQ ID NO: 211) | 7 | 2.22 | 9 | 8.04 | 0.26 | (0.08-0.78) |
| GGCGGC | (SEQ ID NO: 212) | 17 | 5.38 | 4 | 3.57 | 1.54 | (0.47-5.52) |

TABLE 13
FCER2-Clump Identified Risk Haplotypes (Steroid Response)

| | | | | | | |
|---|---|---|---|---|---|---|
| Haplotype | # High | % | # Low | % | OR | CI |

| | | | | | | | |
|---|---|---|---|---|---|---|---|
| GGTCC | (SEQ ID NO: 213) | 9 | 3.98 | 15 | 6.82 | 0.57 | (0.22-1.41) |
| GGCTT | (SEQ ID NO: 214) | 11 | 4.87 | 13 | 5.91 | 0.81 | (0.33-1.33) |
| GTCCC | (SEQ ID NO: 215) | 13 | 5.75 | 12 | 5.45 | 1.06 | (0.44-2.44) |
| CTCCT | (SEQ ID NO: 216) | 14 | 6.19 | 8 | 3.64 | 1.75 | (0.67-4.66) |
| GGTCT | (SEQ ID NO: 217) | 21 | 9.29 | 22 | 10 | 0.92 | (0.47-1.81) |
| GGCCC | (SEQ ID NO: 218) | 22 | 9.73 | 22 | 10 | 0.97 | (0.50-1.89) |
| GTTCT | (SEQ ID NO: 219) | 24 | 10.62 | 10 | 4.55 | 2.5 | (1.11-5.47) |
| AGTCC | (SEQ ID NO: 220) | 27 | 11.95 | 19 | 8.64 | 1.44 | (0.74-2.79) |
| CGCTC | (SEQ ID NO: 221) | 33 | 14.6 | 31 | 14.09 | 1.04 | (0.59-1.83) |
| GTTCC | (SEQ ID NO: 222) | 37 | 16.37 | 44 | 20 | 0.78 | (0.47-1.30) |

Utilizing the preliminary results from the Forest population, 5 genes were selected for replication in CAMP including CRHR1 (corticotropin releasing hormone receptor 1), FCER2 (low affinity IgE receptor), IL18BP (IL18 binding protein), NR3C1 (glucocorticoid receptor), and STAT3 (signal transduction and activator of transcription 3). Since haplotypes are, in general, more powerful to detect associations in candidate genes, our goal was to see if risk haplotypes replicated in our second population. Note that the phenotypes vary between the populations, since CAMP was conducted over a much longer time frame. Since the initial study demonstrated a significant effect with budesonide at one year, but not at four years (28), the difference in FEV₁ between these two time points and baseline formed the basis for our response phenotypes.

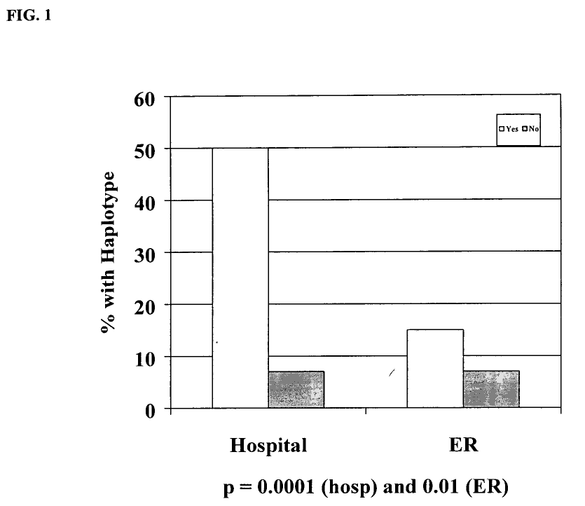

Due to slight differences in genotyping success of these genes in the two populations, we used a haplotype tagging program, BEST (32), to attempt to find common sets of haplotype tagged SNPs in both populations. For all the replicated genes except STAT3, we were able to find a simplified set of haplotype tagged SNPs that allowed for the deliniation of any haplotypes imputed with a frequency of at least 5% in both populations. The results of the haplo.score analysis, including both the Forest and CAMP analyses for CRHR I, FCER2, and IL18BP are shown below (Tables 14-16, FIG. 1). All results are for the Caucasian subjects only and are adjusted for baseline FEV₁.

TABLE 14
CRHR1 Haplo.score results - Corticosteroid
FEV₁ response

| CRHR1 | SNP | Alleles |
|---|---|---|
| | rs1876828 | [A/G] |
| | rs242939 | [A/G] |
| | rs242941 | [G/T] |

| | Phenotype | Frequency | P-Value |
|---|---|---|---|
| Forest | | | |
| Haplotype | | | |
| GAT | % Change | 0.27 | 0.04 |
| AAG | Quartiles | 0.21 | 0.06 |
| CAMP | | | |
| Haplotype | | | |
| GGT | % Change 1 yr | 0.04 | 0.007 |
| AAG | % Change 1 yr | 0.23 | 0.03 |
| GGT | 1 vs 4 yr | 0.06 | 0.01 |
| GAT | 1 vs 4 yr | 0.21 | 0.04 |

Global P value for 1 vs 4 yr

TABLE 15
IL18BP Haplo.score results - Bronchodilator Change

| IL18BP | SNP | Alleles |
|---|---|---|
| | rs1892919 | [T/C] |
| | G9772a3 | [G/A] |
| | G9772a6 | [G/C] |

| | Phenotype | Frequency | P-Value |
|---|---|---|---|
| Forest | | | |
| Haplotype | | | |
| CGG | % Change | 0.04 | 0.001 |
| TAC | % Change | 0.47 | 0.006 |
| TAG | % Change | 0.45 | 0.006 |
| CGG | Extreme BD | 0.07 | 0.00002 |
| TAC | Extreme BD | 0.48 | 0.00003 |
| TAG | Extreme BD | 0.43 | 0.0002 |
| CAMP | | | |
| Haplotype | | | |
| CGG | % Change 1 yr | 0.07 | 0.04 |
| CGG | Quartiles 1 yr | 0.08 | 0.04 |

Global P value for % Change = 0.009
Global P value for Extreme BD = 0.0002
Global P value for % Change 1 yr = 0.03

TABLE 16
FCER2 Haplo.score Results - Pleiotropy of Effects
of Common Haplotypes

| FCER2 | SNP | Allele |
|---|---|---|
| | G9782a12 | [T/G] |
| | G9782a19 | [T/C] |
| | G9782a26 | [C/T] |
| | G9782a5 | [C/T] |
| | G9782a8 | [C/A] |

| | Phenotype | Frequency | P-Value |
|---|---|---|---|
| Forest | | | |
| Haplotype | | | |
| TTCCC | BD % Change | 0.06 | 0.03 |
| TCTCA | BD Extremes | 0.05 | 0.05 |
| GTCTA | Steroid Extremes | 0.13 | 0.02 |
| CAMP | | | |
| Haplotype | | | |
| GTCTA | BD % Change | 0.15 | 0.03 |
| GTCTA | BD Change 1 yr | 0.08 | 0.01 |
| GTCTA | BD Change 4 yr | 0.08 | 0.02 |
| TCTTA | Steroid Ch. 1 yr | 0.04 | 0.04 |
| TTCCC | Steroid Ch. 4 yr | 0.07 | 0.03 |

Global P value for Steroid Change 1 yr = 0.001

The risk haplotypes for CRHR1 in Forest are both also risk haplotypes in CAMP. IL18BP also has one common risk haplotype that replicated. The FCER2 gene supports risk haplotypes that have a steroid effect in one population and a bronchodilator one in the other, likely due to interactions of the glucocorticoid and P2-agonist pathways.

### Example 2

### Genotyping and Analysis of Candidate Genes in Three Study Populations

Materials and Methods

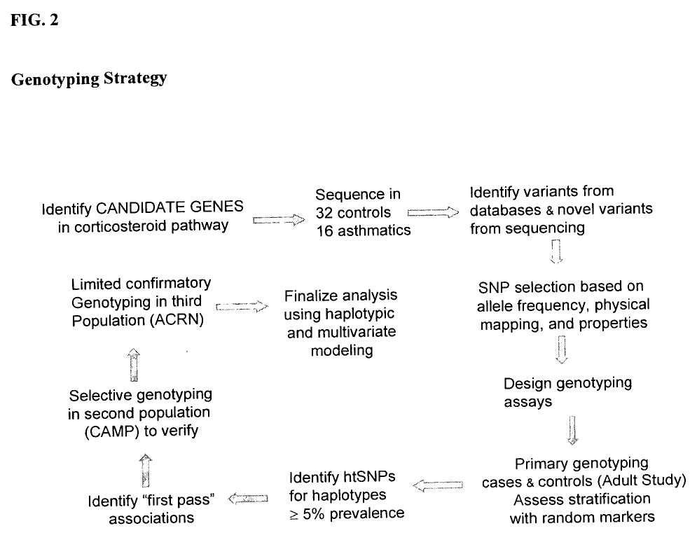

A graphical summary of the approach utilized for genotyping and analyzing candidate genes for the pharmacogenetic response to inhaled corticosteroids is shown in FIG. 2.

Study Populations

DNA samples from three clinical trials were obtained. All patients or their legal guardians consented to the study protocol and ancillary genetic testing.

The Adult Study was a multicenter 8-week randomized clinical trial comparing the effect of once-daily high-dose inhaled flunisolide vs. standard inhaled corticosteroid therapy. Inclusion criteria were a history of asthma, >12% improvement in FEV₁ with albuterol and using inhaled steroids at randomization. Exclusion criteria were non-asthma pulmonary disease, smoking (≧10 pack-years) and recent asthma exacerbations requiring systemic steroids. Subjects were phoned weekly and had spirometry at 4 and 8 weeks.

CAMP is a multicenter, randomized, double-blinded clinical trial testing the safety and efficacy of inhaled budesonide vs. nedocromil vs. placebo over a mean of 4.3 years. Trial design and methodology have been published (27, 28). The replication sample subjects were the Caucasian CAMP children randomized to the steroid group, evaluated at their 2 month follow-up visit.

Two completed trials conducted by the ACRN (Asthma Clinical Research Network), the salmeterol or corticosteroids (SOCS) (54) and salmeterol±inhaled corticosteroids (SLIC)

(55) trials, had a common initial 6-week run-in period utilizing 4 inhalations twice daily of triamcinolone prior to separate randomization to one of the two trials. Details regarding the entry criteria, run-in period and randomization have been published with the primary trial results (54, 55). Of the 339 subjects eligible for randomization, 336 had DNA available; 66.7% of these were Caucasian, forming the basis of the second replication sample.

Genotyping

131 SNPs in 14 candidate genes involved in innate glucocorticoid synthesis and metabolism, cellular receptors, and transcriptional regulators were genotyped (Table 17).

TABLE 17
SNPs Genotyped in the Initial Test Population (Adult Study)

| | | |
|---|---|---|
| Gene | SNP* | Flank |

| | | | |
|---|---|---|---|
| ALOX15 | rs1871346 | TGTGGCTATTTAGAAGTCCAAGGCTGA [C/T] ACCTGATCTTTCGTATGTTTTTCTCTCTCA | (SEQ ID NO: 223) |
| ALOX15 | rs2255888 | GCATAAAAGCCGCTGCCTCCCTGTTG [C/T] CTGCAGAATAAAAGTCCAAATGTTTCTGG | (SEQ ID NO: 224) |
| ALOX15 | rs743646 | AGGAATCGTGAGTCTCCACTATAAGAC [A/G] GACGTGGCTGTGAAAGACGACCCAGAG | (SEQ ID NO: 225) |
| ALOX15 | rs916055 | ACCCAAGCCACAAGCTGACCCCTTCG [C/T] GGTTATAGCCCTGCCCTCCCAAGTCCCAC | (SEQ ID NO: 226) |
| CRH | CRHd3 | TCTCTGCAGAGAGGCGGCAGCACCC [G/A] GCTCACCTGCGAAGCGCCTGGGAAGGTA | (SEQ ID NO: 227) |
| CRH | CRHd4 | TCGCCCGCCTCCTCGCTCCTCGCCGG [A/C] GGCAGCGGCAGCCGCCCTTCGCCGGAA | (SEQ ID NO: 228) |
| CRH | rs1870392 | TCTCATAGAAATGAGAGGTAACACA [C/G] AAGCATTTTGGAAAGACCCCTCTGAATGC | (SEQ ID NO: 229) |
| CRH | rs1870393 | CATCGCTGTCCACGGTTTGGTGGGGA [A/C] AGTTCCCCATCAATCAATCAAAAATTCTGTCAG | (SEQ ID NO: 230) |
| CRHBP | rs1505079 | GAAAACTTATCAGTCAAGTCTTTGGTA [A/G] TATAATTTTATCTTAAATGCTTCTAAAATGT | (SEQ ID NO: 231) |
| CRHBP | rs1700676 | CATAAGAAACTCCATTTTGACCTGTAC [C/T] CTGAACAATTGCTTTGCCCTGAGATGCTG | (SEQ ID NO: 232) |
| CRHBP | rs1715771 | CCCGCGTCCCGGGCGCCCGCGAGCC [C/G] GGCAGCCTCGACTCACGCAGAGCGCGG | (SEQ ID NO: 233) |
| CRHBP | rs2135078 | ATTATTTAGAGGAGAGGTAGAATTGCA [A/G] TGTTTGCACAGGCAACACTAGCTGGTCCT | (SEQ ID NO: 234) |
| CRHBP | rs247742 | TCAGTCAACAGCCCATCAACATCAAA [C/G] ACTCACCACCAGCAAAAAGATTATGACTC | (SEQ ID NO: 235) |
| CRHR1 | rs1396862 | GAGCTTGGTTTTAGGAAAAAGCACCT [C/T] TGCAGTTCAGAAGCCCTGGTCCAACCACC | (SEQ ID NO: 236) |
| CRHR1 | rs171440 | GTCCCCTGCTCTGTAGCCTAAGGACA [C/T] TTCTCTTGGTCCCTCGCATGGTGACAGCC | (SEQ ID NO: 237) |
| CRHR1 | rs171441 | ATCTACCCTGGCCCTGCAGGGAAGAA [C/T] CAGCTAAATGAAGTTGGCCCTCCTTCCG | (SEQ ID NO: 238) |
| CRHR1 | rs1876827 | GGGATGACACTCACAGCCTTAACACG [A/G] CTGCTTTGCATATTTGTCGGAACAGGTTTC | (SEQ ID NO: 239) |
| CRHR1 | rs1876828 | GCAGCATACCCCTAGGGACCTAGGA [A/G] CAGGGAGGGAGAGAGGCAGCCCTGGGA | (SEQ ID NO: 240) |
| CRHR1 | rs1876829 | GTCCCAACAGGCCTCACAGCCCTGA [A/G] CCCCGCTGCAGGGCCCCCGGGTCCTCAC | (SEQ ID NO: 241) |
| CRHR1 | rs1876831 | CCCCAACCAGAGATGATGATGGGGG [A/G] CAGGGGAGGCACCAAACCCTGGGCCTGG | (SEQ ID NO: 242) |
| CRHR1 | rs242924 | AAGACACTCAGGTGCAGGGACCCTCT [A/C] CATTTTTGCCCAGCAGCAGCCATGCCCAG | (SEQ ID NO: 243) |
| CRHR1 | rs242936 | ATTGCTACCTCCATCCCGTCCCTGGTT [C/T] CCATACAGCCCTGTGGCTGGAACTGGATG | (SEQ ID NO: 244) |
| CRHR1 | rs242938 | TCCTTTCCTGGGATCACAGAGGGAAG [C/T] GCGGGGGAGCCTAGAGAGCACCACACTC | (SEQ ID NO: 245) |
| CRHR1 | rs242939 | GAACACGGAGGCCACACAAGAGTGG [A/G] TTCCAAGTGAAGGAGTGACCAACTCAGA | (SEQ ID NO: 246) |
| CRHR1 | rs242940 | GGCACACCAGTCCTTTTGAGCCCCAG [C/T] GTCCCCAGGTTAATAACCTAGAATTGGCA | (SEQ ID NO: 247) |
| CRHR1 | rs242941 | GGGCCAGGAACCATGAACCAGCGCG [G/T] GTGGGGGCAGCCTCTTCAGGCCTGGGCC | (SEQ ID NO: 248) |
| CRHR1 | rs242949 | GAGCCCTGGGCAGGGGATACATGTGG [G/T] TTGAGGGCAGGGAGCCTTCATGGCAAA | (SEQ ID NO: 249) |
| CRHR1 | rs242950 | GGTGGCCCCCACCTCTAGGTAGAGG [A/G] GTCCTTTCTGTCCACGGTTGGCACTGATTG | (SEQ ID NO: 250) |
| CRHR1 | rs739645 | TCTTTCCTCTACCAGATGGATTTGGGG [G/T] GTTAAGGTTGGGGGCTACAGCAGAGGAG | (SEQ ID NO: 251) |
| CRHR1 | rs81189 | GAGTCCCAAGAGGGCACAGGGGTGA [C/G] CCCAGACACCATGTAGTTTACTCCAAGA | (SEQ ID NO: 252) |
| FCER2 | G9782a10 | CACCAGCTGTGTCTCCCTGCTAACCA [C/T] GCTAGTGAGTCCAGATTGTAGACTAAACA | (SEQ ID NO: 253) |
| FCER2 | G9782a12 | GAAGCTGGGGGCCTGGCATTGGTTGT [T/G] GGGGCTGAGGGAGTCTTAGCTCTTAGTC | (SEQ ID NO: 254) |
| FCER2 | G9782a13 | ATCTGTCTCTGTGGNCAGTGACCCAGC [C/T] CTGAGTCAGGTAAGGAAGCTGTGCAAAT | (SEQ ID NO: 255) |
| FCER2 | G9782a15 | TTCCCAGGAGGNGGTGTTGCAGGCG [T/C] GGGACTCAGATCGTGCTGCTGGGGCTGGT | (SEQ ID NO: 256) |
| FCER2 | G9782a17 | ATAGCATCCTAATACAGATGCTCTTCC [G/T] CTTGCAATGGGGTTATGTCCCCATAAGC | (SEQ ID NO: 257) |
| FCER2 | G9782a19 | GTTGGTCTCTGAGCACCGCCCCTTGT [T/C] GACTCCCCAAGAATTGAACGAGAGGAAC | (SEQ ID NO: 258) |
| FCER2 | G9782a22 | CCACAGCCCGGAGGAGCAGGTGGGC [T/C] GGGGCTCTGCAGAGGTGGTGGGCAGC | (SEQ ID NO: 259) |
| FCER2 | G9782a26 | GAGTTTATCTGGGTGGATGGGAGCCA [C/T] GTGGACTACAGGTGAGGAGGGGGCCTC | (SEQ ID NO: 260) |
| FCER2 | G9782a5 | GATGGCTCACCCTAACCATCATTAAAT [C/T] CCAAATCAGCCAGAGCTGTGATTGTGCC | (SEQ ID NO: 261) |
| FCER2 | G9782a8 | CTGGCACAGAGCCAGGAAGGAGTGG [C/A] AAATTGAGGGCCCCTCCTTTTTCTGATTC | (SEQ ID NO: 262) |
| FCER2 | rs1042428 | ACACGTGCCCTGAAAGTGGATCAA [C/T] TTCCAACGGAAGTGCTACTACTTCGGCAAG | (SEQ ID NO: 263) |
| FCER2 | rs1990975 | CAGACCTGAGTCCGAGTCCTGGCTTCT [C/T] CCTGGATGAGCAGCTCAGTTTGCTCATCT | (SEQ ID NO: 264) |
| FCER2 | rs6952 | GCGTCTTCTCCGTGGCCGAGCTGCAG [G/T] CGCGCCTGGCCGCGCTGGGCCGCCAGG | (SEQ ID NO: 265) |
| FCER2 | rs753733 | GAGAGTGGGGAGGGTGTTAAGGATCA [A/G] GGGACACATTTTGGGAAGGATGAGGAG | (SEQ ID NO: 266) |
| FCER2 | rs889182 | TGCCGGGCTGCGCACAGTGGGTCTTC [A/G] GATTCTAAACATTTATGAAACATCTACTCT | (SEQ ID NO: 267) |
| GATA3 | G9779a6 | ACAACACATTTAACATTTGTTTTGATTT [C/T] ACCCTCTCCTCTCTCCCCACTCTCAGTCTG | (SEQ ID NO: 268) |
| GATA3 | rs1399180 | AAGGCAATTCCAGTACCACCTCTTTC [C/T] CCCTTTCACCTGGAGAAGTTCAGGAGAGT | (SEQ ID NO: 269) |
| GATA3 | rs2228254 | ATGAAGCTGGAGTCGTCCCACTCCCG [C/T] GGCAGCATGACCGCCCTGGGTGGAGCCT | (SEQ ID NO: 270) |
| GATA3 | rs2229360 | GCAGGAGCAGTATCATGAAGCCTAAA [C/T] GCGATGGATATATGTTTTTGAAGGCAGAA | (SEQ ID NO: 271) |
| GATA3 | rs403029 | GAGTCCCCCTCCCCCTCTTTTCCTATCC [C/G] TGCTGTGAACACATCCCCTGCCAGAGTG | (SEQ ID NO: 272) |
| GATA3 | rs412359 | CCCCCGACCTCCCAGGCGGACCGCC [C/T] TCCCTCCCCGCGCGCGGGTTCCGGGCCC | (SEQ ID NO: 273) |
| GATA3 | rs422628 | GACAACACATTTAACATTTGTTTTGATTT [C/T] ACCCTCTCCTCTCTCCCCACTCTCAGTCT | (SEQ ID NO: 274) |
| GATA3 | rs520507 | AGAAGATGCGGGCAGCCTGGCTGGCC [C/G] AGGAGAGACGAGTGGTCAGAGAATGA | (SEQ ID NO: 275) |
| GATA3 | rs9746 | GAAAAAAGAGAAAAGAAAAAAAAAG [A/G] AAAAAGTTGTAGGCGAATCATTTGTTCAA | (SEQ ID NO: 276) |
| HSD11B1 | rs1000283 | ATCTCCCCCAAATACTTAGTGTTCATTT [C/T] TTACACAGAGGGACACTGTCCTACATAAT | (SEQ ID NO: 277) |
| HSD11B1 | rs1337531 | TGTCCCCAAAAAAGACATATTAAGTC [G/T] CTAATCCCTAAAACCTCAGAACATGATTTTA | (SEQ ID NO: 278) |
| HSD11B1 | rs1415542 | CCCATATCAATGCACAAAAAATGTGT [C/T] GGAAGAAAAAGAGGATTGCTACTCTCAGA | (SEQ ID NO: 279) |
| HSD11B1 | rs1474654 | TTCATAAGACAGTGCTCTGTGGGAAA [A/G] CTCAAACCAACAGATGAAGTCACTTGCTG | (SEQ ID NO: 280) |
| HSD11B1 | rs1474655 | TTATGAACGGGCCTCAGAACCAGAGT [C/G] GAAAGGGGCAATTTATCCCTTGCCACAG | (SEQ ID NO: 281) |
| HSD11B1 | rs2282739 | ACCACCATTCCTAGAGTGCTTGTTTACA [C/T] CTGCATGTCCAAGATGGCTCATTGGCAT | (SEQ ID NO: 282) |
| HSD11B1 | rs846906 | GAAAGCTGTTGAAATACAGCATTTTAC [C/T] CACAGTGATTAGGCTGTGCTCTCCTTAAAG | (SEQ ID NO: 283) |
| HSD11B1 | rs846911 | TAGCAATTATTTTATCTAATCCCATGAAA [A/C] TCACTTTATTGGATGCTTTTGCCATATCC | (SEQ ID NO: 284) |
| HSD11B1 | rs860185 | GTTCTCTGTTAAATAATAAGTGTATGA [A/T] CCATTGCACTTGCCTTAGAGTCTTGAACC | (SEQ ID NO: 285) |
| HSD11B1 | rs932335 | AAAGCCTACTACAGCTCTGTAAGAAG [C/G] TGAAATGGGCAGCCTTATTAACCCATTTC | (SEQ ID NO: 286) |
| IL18BP | G9772a3 | CATGGGGACTGGGGGGAGCTGGCAG [G/A] GAGGGCACAGCAGAGCAGGGTAGGG | (SEQ ID NO: 287) |
| IL18BP | G9772a6 | GACACCAGGTAGGCCTTGGGGCTAC [G/C] CATGGGCAGGCGGGGTAGGGTGAGGTC | (SEQ ID NO: 288) |
| IL18BP | rs1541304 | GCTCTTTCCCAGGACGGATGGGCCCT [A/G] TGTCTCAGGAGTGGGGTTGGGGGACAG | (SEQ ID NO: 289) |
| IL18BP | rs1573503 | CGGTGACTTGGGAGCCCAGGTGACA [A/G] AGGCAGTGCTGGATGGCTGCTGCTCCTC | (SEQ ID NO: 290) |
| IL18BP | rs1892919 | TCCCCTACCCTGCTCTGCTGTGCCCTC [C/T] CTGCCAGCTCCCCCCAGTCCCCATGCATC | (SEQ ID NO: 291) |
| IL18BP | rs2155145 | TAGATATCTGGTATAATACCCGTTTTTC [G/T] TTATTCTTCTCTAAGAATAAATTTAGATCAC | (SEQ ID NO: 292) |
| IL18BP | rs949323 | TGGCCCCACCTGTGTCCCCGATGCTG [A/C] CCTCACCTGGTCCTCCGCCTACTGTCCCTC | (SEQ ID NO: 293) |
| MAPK8 | G1096a3 | ATCTAGCAGTCTGTGTTACTATCAGTA [C/A] GTAAACAGTAAGGACTCAAATTTTAAGAT | (SEQ ID NO: 294) |
| MAPK8 | rs2698762 | TGTGTTATACTATGCTATATCATATATA [A/C] TATATAATCTAATTATCCTCCCTTGACCATT | (SEQ ID NO: 295) |
| MAPK8 | rs724124 | GAATGTGGAACAACTGGACCTCTCACA [C/T] GTTGCTGGTGCAAATGAAAAATGATATG | (SEQ ID NO: 296) |
| MAPK8 | rs9284 | CCCCAGAGGAGTGAGGGAAAATAAC [G/T] TGTAGCCAGTTATATTCAGGAATAACTACT | (SEQ ID NO: 297) |
| NFATC4 | G3141a10 | TTTGGGGGCTACAGAGAAGCAGGGG [G/C] CCAGGGTGGGGGGGCCTTCTTCAGCCCA | (SEQ ID NO: 298) |
| NFATC4 | G3141a14 | CAGTACTCATCATGAGGGGCCAAGGG [G/T] TGAATGGAACCTGGGAGGAGCAGGCAG | (SEQ ID NO: 299) |
| NFATC4 | G3141a16 | CGTATGGAGGGCGGGGCTCCTCTTTC [T/C] CCCTGGGGCTGCCATTCTCTCCGCCAGC | (SEQ ID NO: 300) |
| NFATC4 | G3141a17 | GCAACCCCAGCCCCAGCCTCAGCCCT [G/T] CCCCCTTTCCCTCCTTCCTGGAGTGGTG | (SEQ ID NO: 301) |
| NFATC4 | G3141a5 | TTTGGGGGCTACAGAGAAGCAGGGG [C/G] CCAGGGTGGGGGGGCCTTCTTCAGCCC | (SEQ ID NO: 302) |
| NFATC4 | G3141a8 | GTGGAGCTTCTTCTCCGATGCCTCTGA [C/T] GAGGCAGCCCTGTATGCAGCCTGCGAC | (SEQ ID NO: 303) |
| NFATC4 | G3141u4 | GTGGAGCTTCTTCTCCGATGCCTCTGA [C/T] GAGGCAGCCCTGTATGCAGCCTGCGAC | (SEQ ID NO: 304) |
| NFATC4 | rs10362 | CAACCCCAGCCCCAGCCTCAGCCCT [G/T] CCCCCTTTCCCTCCTTCCTGGAGTGGTGGC | (SEQ ID NO: 305) |
| NFATC4 | rs1950500 | TTCTGTGAGGTCTGCCTTTATAATATTC [C/T] TCTTTTGCTTAAGTTACAAGAAGTCAGTTTG | (SEQ ID NO: 306) |
| NFATC4 | rs1955915 | TGGCTATGTTCCTTGAGTGGCCAGGCC [A/G] CAAGTCCTTCTATGCTCCCTGCCCCTCAG | (SEQ ID NO: 307) |
| NFATC4 | rs2228233 | GTGGAGCTTCTTCTCCGATGCCTCTGA [C/T] GAGGCAGCCCTGTATGCAGCCTGCGACG | (SEQ ID NO: 308) |
| NR3C1 | GRLd21 | ACTGTAGCTGTAGGTGAATGTGTTTTT [G/T] TGTGTGTGTGTCTGGTTTTAGTGTCAGAAG | (SEQ ID NO: 309) |
| NR3C1 | rs1438732 | AAATTTTTAGGGACTTTCAAAAACTCA [C/G] ACTCTTGGGTTCTGACCCTGTAACTCTTAA | (SEQ ID NO: 310) |
| NR3C1 | rs1866388 | AAATATTTAACAAATCCTTAATTATTTG [A/G] CTTAAATTTGCAAAGTAAGACTGAAAAAT | (SEQ ID NO: 311) |
| NR3C1 | rs258750 | TGGATTAATCATACTTTTTAAAAACAGT [A/G] TTACTAAATTCTGTAATAACATGGTGATT | (SEQ ID NO: 312) |
| NR3C1 | rs33388 | TGAAAGTCATGGATGGATTATGAGTTA [A/T] TCACACACCTAGAGAAGCATGTAAAATGT | (SEQ ID NO: 313) |
| NR3C1 | rs33389 | GTTTGCTCAGGCTTGCATTAGGGGATG [C/T] GAGTTTTAAGCAGAAGCAATAATAGTACA | (SEQ ID NO: 314) |
| NR3C1 | rs6188 | TTCCCATTACAGTTCATTTCTATGTATTT [G/T] TTTAAATACCCACAGCTCGAAAAACAAAG | (SEQ ID NO: 315) |
| NR3C1 | rs6191 | TGACACTAAAACCAGACACACACACA [A/C] AAAAACACATTCACCTACAGCTACAGTCA | (SEQ ID NO: 316) |
| NR3C1 | rs6195 | ATCTCCAGATCCTTGGCACCTATTCCAA [C/T] TTTCGGAACCAACGGGAATTGGTGGAAT | (SEQ ID NO: 317) |
| NR3C1 | rs6196 | GATGAAACAGAAGTTTTTTGATATTTCC [A/G] TTTGAATATTTTGGTATCTGATTGGTGATG | (SEQ ID NO: 318) |
| NR3C1 | rs852975 | TATACCTAGAAAACCCTGAAGACTCTG [C/T] CAAAAGGCTCCTGGAGCTGATAAACAAC | (SEQ ID NO: 319) |
| NR3C1 | rs852977 | CTTCTGTGTGCATTTTTTAGTTAATCTCT [A/G] CAGTTTTTATAACATTTACAAGAAAGTGG | (SEQ ID NO: 320) |
| NR3C1 | rs852978 | TGTATCAGGTTCAATTCTTTGTAAAGAA [C/T] AGGCCACAAAATTGACCACTAGACTATA | (SEQ ID NO: 321) |
| NR3C1 | rs852979 | TCTATTTTATTCTAGATCTTTTTGTATTGT [C/T] GTTTTAAATACTTTCCTGCCCATTAGAGGA | (SEQ ID NO: 322) |
| NR3C1 | rs852983 | GTTGATAGGTACAGCAAACCACCATG [A/G] CCACATGTTTACCTATGTAACCTGCAAATC | (SEQ ID NO: 323) |
| NR3C1 | rs860457 | TGGCCCTATGCCCTCTATGGTGTGCCA [C/T] GCTATTTTGTGACTGACTCTGCAACCTAA | (SEQ ID NO: 324) |
| POMC | rs1009388 | AGGGAGCCGGCGGCCTCCTCTCCCC [C/G] AGGGGCTCGCGGCGGTCCGGAGGCTCC | (SEQ ID NO: 325) |
| POMC | rs1042571 | AGGTCGACCCCAAAGCCCCTTGCTCT [C/T] CCCTGCCCTGCTGCCGCCTCCCAGCCTG | (SEQ ID NO: 326) |
| POMC | rs1866146 | GGTGGAGTCAGGTGAATGGATAAGA [A/G] GCAGATCGGCAGAAAGCATCAGTGTGGT | (SEQ ID NO: 327) |
| POMC | rs2028195 | ACTCTGTCTCAGAAAAAAAAAAAAAAA [G/T] TTAACCAGCAGCCCTCCAGGTCGCTCTGC | (SEQ ID NO: 328) |
| POMC | rs2071345 | CGGCCTGGGCCCCTGCGCCGTCATC [A/G] GCAGGGCCGTCGGGGCCATCTCCCTCCCG | (SEQ ID NO: 329) |
| POMC | rs934778 | GTTCTGGCTGTGTACTTGAATAGATCAC [C/T] GGCAGGGTACAATGGGAACAGCCTGTC | (SEQ ID NO: 330) |
| STAT3 | G3363a16 | GGGAAAATGAGATCAGGAGATAAAG [G/T] GGCACCCTTTGGTCTTGTAAAGCCTTTTTTA | (SEQ ID NO: 331) |
| STAT3 | G3363a3 | ACAGACATCATTTGAACTAGAGACTCT [G/A] TCTTTATTCAGAGATCTTCATTTTGTGGAC | (SEQ ID NO: 332) |
| STAT3 | G3363a4 | TCCCCTTCACAAAGGGCCTCTGGCTGC [C/G] GGAGAGGGCTAGGGAGAGCCTCACAG | (SEQ ID NO: 333) |
| STAT3 | rs1026916 | AGGAAAAAGTTTAACCCAAAGACTGT [A/G] TGGATCTTCTCTACCCTACATCTCCAATCT | (SEQ ID NO: 334) |
| STAT3 | rs1905340 | TATTTGAGAATCTAAGAAAGTAGATCA [A/C] ACTAAATATTGATATGCAGACACTAAAATC | (SEQ ID NO: 335) |
| STAT3 | rs1963987 | TAAAAGAGGCTGGGTGCAGTGGCTCA [C/T] GCCTGTAATCCCAGAACTTTGGGAGGCC | (SEQ ID NO: 336) |
| STAT3 | rs2230097 | CGACCTCTCCATCTTCAGCTTCTTCATC [C/T] TCACCAGAGGAATCACTCTTGTGGATGTT | (SEQ ID NO: 337) |
| STAT3 | rs2354155 | TGCTGGCATTACAGGCGTGAGCCACC [A/G] CTCCCGGCCTTTTTTGTTTTTTGAAACCAA | (SEQ ID NO: 338) |
| STAT3 | rs744166 | AAACTGTTTGTTCTATAAATTACTGTCA [A/G] GCTCGATTCCCTCAAGACATTACAGCCAC | (SEQ ID NO: 339) |
| STAT3 | rs957971 | TGTTATATGAAGTGAATTAACCTCCTAT [C/G] GTACTTCAGTTTTCTCTATGCTAAAAGTGT | (SEQ ID NO: 340) |
| STAT5A | G3469a11 | AGTTTGGGGTTTGGGGTTTGGGGTCTG [T/C] AGTATTGGTGTTTCCTAATGCCTGTGGTCT | (SEQ ID NO: 341) |
| STAT5A | G3469a13 | GGGGAACGGGAGCTGTGTCTTGGGG [C/A] CTGGCGTCTGTGAGGAGAAGCCATTGTC | (SEQ ID NO: 342) |
| STAT5A | G3469a15 | TCAGGGGCCAGCTGTGGGCGCAGAG [A/G] GACTGTGGCTGTGGCCCAGTGGTGACG | (SEQ ID NO: 343) |
| STAT5A | G3469a17 | GGCATCACCATCGCCTGGAAGTTTGA [C/T] TCCCGTGAGTGCCCGTTTTGCCCACACTC | (SEQ ID NO: 344) |
| STAT5A | G3469a18 | AGGTGATGTGAGCAGGAGGGAGACT [A/G] CATGGGGCGTGGGNTTCCACCCCACTTG | (SEQ ID NO: 345) |
| STAT5A | G3469a19 | GGAGGGAGACTNCATGGGGCGTGGG [C/T] TTCCACCCCACTTGGGAGTTCCCAGAGA | (SEQ ID NO: 346) |
| STAT5A | G3469a9 | ATGAGCCTGGGGTTTCCACTTTATTCC [A/G] GCTCCCTGACCTCCTTGCCCAAGGAGGT | (SEQ ID NO: 347) |
| STAT5A | rs2883375 | CTGCAACCTCCACCCCTTGGGTTCAAG [A/C] GATTCTCATGCCTCAGCCTCCCAAGTAG | (SEQ ID NO: 348) |
| STAT5A | rs2948176 | CAGCTCCTGCCTGCGTGGGGGGAGC [C/T] GCAGGTGCCTTCCAGACCAGCAGATCCA | (SEQ ID NO: 349) |
| STAT5A | rs909056 | GGAGAAGAGAGGAGGGGAGGGGAC [C/G] GGCAGGTGCCACCGCCCCAGGGGGCTA | (SEQ ID NO: 350) |

*SNPs obtained uniquely through the Whitehead sequencing efforts are designated as “G” SNPs.

SNPs were selected utilizing two sources, public databases and cDNA sequencing performed at the Whitehead Institute. Exonic and promoter regions were over-sampled and coverage of at least one SNP every 10 kb was attempted. Replicate genotyping was performed in CAMP on three candidate genes with a measurable effect in the Adult Study and in ACRN on three htSNPs of the single gene with associations in both the Adult Study and CAMP.

SNPs were genotyped via a SEQUENOM MassARRAY MALDI-TOF mass spectrometer (Sequenom, San Diego, Calif.) for analysis of unlabeled single-base extension minisequencing reactions with a semiautomated primer design program (SpectroDESIGNER, Sequenom). The protocol implemented the very short extension method (33), whereby sequencing products are extended by only one base for 3 of the 4 nucleotides and by several additional bases for the fourth nucleotide (representing one of the alleles for a given SNP), permitting clearly delineated mass separation of the two allelic variants at a given locus.

Statistical Methodology

The FEV₁ phenotypic measures in the populations reflected similar outcomes over similar time frames. In the Adult Study, associations between individual SNPs and asthma phenotypes were tested with analysis of variance. Genes with significant associations (p-value<0.05) were genotyped in CAMP and tested for associations.

For the Adult Study and CAMP, haplotypes were inferred using the program Phase (29) and the haplotype-tag approach (32) was used to identify the haplotypes with >5% frequency. The minimal subset of htSNPs that was identical for both Adult Study and CAMP were chosen. It was noted that the common haplotypes, although differing in frequency, were represented in both populations, which allowed for the comparison of haplotype-specific effects across the two populations. These SNPs were tested for haplotype association using the Haplo.score program (31). Haplo.score permits analysis of continuous and categorical phenotypes, with and without covariate adjustment. Score tests, derived from generalized linear models, were used for global tests of association, as well as haplotype-specific tests. Linkage phase ambiguity (inherent in methods that infer haplotypes from unphased marker data) was addressed by computing the weighted conditional distribution of haplotypes given the observed genetic data for all study subjects. The method was modified to include data from individuals with partially missing marker information. Given replication in two asthmatic populations, the htSNPs were tested in the ACRN population. Multivariable individual SNP and haplotypic analyses adjusted for age, sex, and baseline FEV₁ were performed for any significant, unadjusted association. Height was also incorporated into the multivariable models involving the CAMP and ACRN populations. In a separate analysis of a random panel of 59 SNPs across the genome in each of the three populations, no evidence of population stratification (p>0.05 for dichotomizations of each study into highest and lowest quartiles) was found.

Results

The association of sequence variants in genes controlling the pharmacokinetics (uptake, synthesis or degradation) or pharmacodynamics (site of action) of corticosteroids with the therapeutic response to this class of asthmatic drugs was tested. Using three clinical therapeutic trials of asthmatics on inhaled corticosteroids, a pathway candidate gene association study was undertaken. The association between single nucleotide polymorphisms (SNPs) in the genes and the longitudinal response to inhaled corticosteroid treatment, measured as the change in forced expiratory volume at one second (FEV₁), was analyzed. The FEV₁ is a standardized and widely accepted measure of lung function; increased FEV₁ indicates improved lung function. A population of adult asthmatics (Adult Study) formed the initial test population, while childhood (CAMP) and second adult (ACRN) populations formed two replication groups.

Clinical characteristics of the primary and replicate populations are shown in Table 18. Due to concerns about possible population stratification, all of the analyses were confined to Caucasians. In addition to age, type of inhaled steroid, and mean duration of follow-up, the mean FEV₁ upon enrollment differed, with the two adult populations composed of moderate-to-severe asthmatics and the pediatric population mild-to-moderate asthmatics.

TABLE 18
Population Characteristics*

| | Adult Study | CAMP | ACRN (Second |
|---|---|---|---|
| | (Primary) | (Replicate) | Replicate) |
| N | 470 | 311 | 336 |
| Inhaled | Flunisolide | Budesonide | Triamcinolone |
| Corticosteroid | | | |
| Used | | | |
| Age | 39.4 ± 13.4 | 9.0 ± 2.1 | 33.2 ± 11.6 |
| Sex - n (%) | | | |
| Male | 195 (41.5) | 181 (58.2) | 139 (41.4) |
| Female | 275 (58.5) | 130 (41.8) | 197 (58.6) |
| Race - n (%) | | | |
| Caucasian^{†} | 415 (88.5) | 201 (64.6) | 224 (66.7) |
| African American | 34 (7.0) | 44 (14.1) | 63 (18.8) |
| Hispanic | 12 (2.6) | 32 (10.3) | 25 (7.4) |
| Other | 9 (1.9) | 34 (10.9) | 24 (7.1) |
| Mean | 72.2 ± 16.2% | 93.6 ± 14.4% | 77.8 ± 15.9% |
| Baseline FEV₁^{‡} | | | |
| Mean Change | 7.0 ± 19.3% | 8.3 ± 14.1% | 6.7 ± 19.7% |
| in FEV₁^{§} | | | |

*Plus-minus values are means ± standard deviations
^{†}Due to concerns over possible population stratification, only genotypic information from Caucasians were analyzed
^{‡}As a percent of predicted
^{§}Change in FEV₁ while on inhaled corticosteriods evaluated at 8 weeks in the Adult Study and CAMP and 6 weeks in ACRN

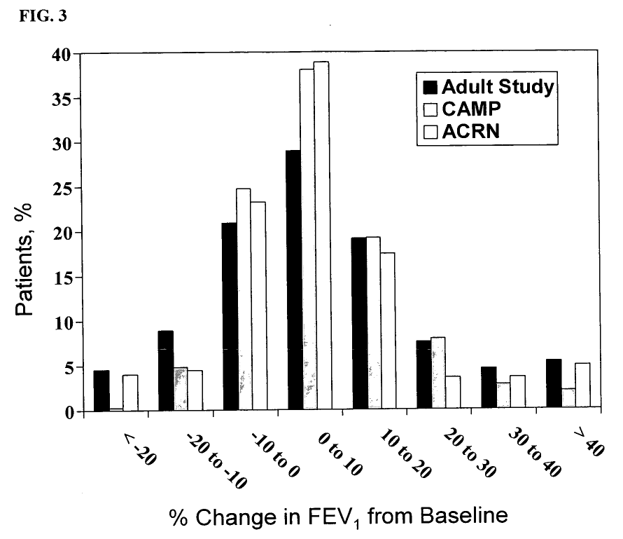

The primary outcome measure of the association analyses was percent change in FEV₁ over time in response to inhaled steroids, defined as the FEV₁ difference from baseline to eight weeks for the Adult Study and CAMP, and to six weeks in ACRN, divided by the baseline value. Although all three studies demonstrated significant improvements over those time frames (p<0.05), there was wide interindividual variability in these responses (FIG. 3).

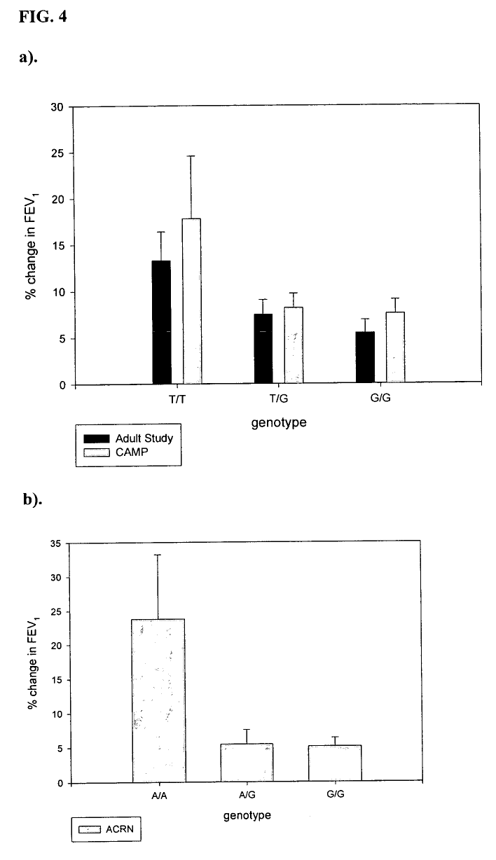

In the Adult Study, 131 SNPs in 14 genes were genotyped (Table 17) and three genes, CRHR1, FCER2, and NR3C1, were identified as being associated with response to inhaled steroids. Sequence information for these genes can be found in GenBank according to the following Accession numbers: NM_{—}004382 (CRHR1 mRNA); AF488558 (CRHR1 gene, promoter region and partial coding sequence); NM_{—}000756 (CRH mRNA); NM_{—}002002 (FCER2 mRNA); NM_{—}000176 (NR3C1 mRNA). Upon genotyping these three genes in CAMP, variation in one CRHR1 SNP, rs242941, was associated with significantly improved lung function after eight weeks of inhaled corticosteroid therapy in both the Adult Study and CAMP (p=0.025 and 0.006, respectively) (FIG. 4A). In the Adult Study, the mean percent change in FEV₁ for those homozygous for the variant allele was 13.28+3.11, compared to 5.49±1.40 for those homozygous for the wild-type allele. Similarly, in CAMP, the percent change was 17.80+6.77 vs. 7.57±1.50 for the variant and wild-type homozygotes.

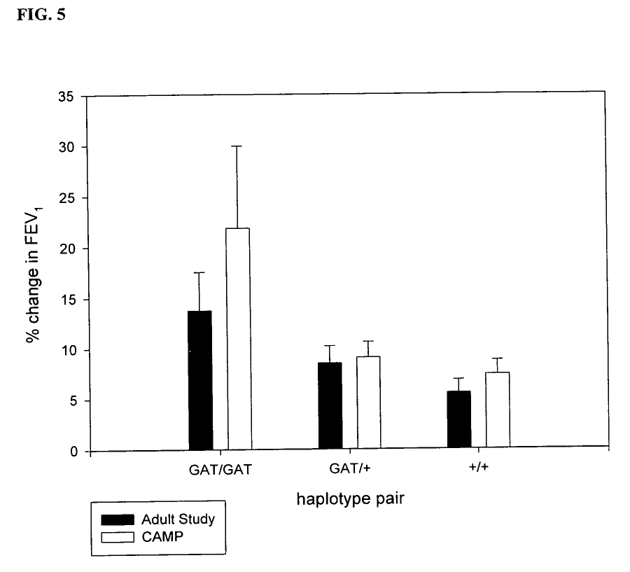

The association at the haplotype level was also tested. Due to linkage disequilibrium and/or limited haplotype diversity, haplotypes may be identified with a relatively small subset of SNPs, termed ‘haplotype-tag SNPs’ (htSNPs) (37). It was found that the htSNPs rs1876828, rs242939, and rs242941 defined four haplotypes imputed with at least a 2.5% frequency in both the Adult Study and CAMP populations. These SNPs were in Hardy-Weinberg equilibrium in all study cohorts. Haplotypic analysis revealed one common CRHR1 haplotype (frequency 27%), termed GAT, associated with a significantly enhanced response to inhaled corticosteroids in both the Adult Study and CAMP (p=0.02 and 0.01, respectively). The estimated short term improvement in FEV₁ for those subjects imputed to have the homozygous GAT/GAT haplotype was over twice that for those homozygous for non-GAT haplotypes in the Adult Study (13.73±3.80% vs. 5.54±1.29%), and nearly three times that in CAMP (21.83±8.07% vs. 7.35±1.41%) (FIG. 5).

To further verify the findings, the role of the three htSNPs in CRHR1 in the ACRN population was evaluated. Over the six-week period, one SNP, rs1876828, was strongly associated with the change in FEV₁ (p=0.006) (FIG. 4B). Subjects homozygous for the variant allele had an average increase in their FEV₁ of 23.72±9.75% compared to 5.14±1.31% for those homozygous for the common allele.

Corticotropin releasing hormone (CRH) is a well-recognized neuroendocrine mediator of the immune system response to stress. CRHR1 is the predominant CRH receptor in the pituitary gland, mediating the release of ACTH (43, 44) and the catecholaminergic response to CRH (45, 46). Peripherally, CRH may bind to mast cells via CRHR1 (47). The improvement in lung function consistently associated with the variant allele in each of the single SNP associations in all three of the populations, shows that alterations of any of the CRH effects, as mediated by the CRHR1 gene, can influence the pathogenesis of asthma.

None of the htSNPs were coding and hence, it is likely that the SNPs identified are in linkage disequilibrium (LD) with the actual disease modifying variants in CRHR1. CRHR1 has at least three known isoforms arising from alternative splicing (48, 49). Within the receptor, there are multiple conserved regions that are important for optimal binding of CRH and activation of the receptor (50, 51). Additionally, CRHR1 translation may be inhibited by an upstream regulator (52). Linkage disequilibrium between any of the alternative splice sites, regulatory regions, or coding regions and our htSNPs could explain the variability noted in the response to inhaled corticosteroids in our populations.

Although an effect of CRHR1 genetic variation on pulmonary function response to inhaled corticosteroids in all three of our asthmatic populations was observed, the strongest effects were observed in the pediatric population. Age, prior steroid use, and marginal sample size leading to reduced power all may contribute to the weaker effects observed in the two adult populations.

The findings of an association of CRHR1 genetic variants with the enhanced response to inhaled corticosteroids in three diverse asthmatic populations provide novel insights into the therapy of asthma. This genetic association with a therapeutic response to this class of commonly used medications is an important step in the development of individualized therapy for asthma, providing a venue to decrease both morbidity and cost. These findings may also be of relevance to multiple other diseases whose therapeutic approaches include the utilization of corticosteroids.

### Example 3

### Genotyping and Analysis of CRHR2

The CRHR2 gene was genotyped after our positive findings with CRHR 1. Of the 17 SNPs genotyped, one (which is unique, from a linkage disequilibrium perspective) was significantly associated with 8 week change in FEV1 in the Adult Study (Forest). The raw p-value for the association was 0.05, but the adjusted (same as in our other Adult Study analyses) p value was 0.01. There is preliminary evidence of haplotypic associations with this gene, as well as associations with baseline lung function. The specific SNP that was associated with 8 week change in lung function was rs255102 (SEQ ID NO: 351). On average, those homozygous AA for that SNP had a 4.4% greater 8 week increase in their FEV1 compared to those homozygous TT.

### REFERENCES

1. Gergen P J, Mullally D I, Evans R, 3rd. National survey of prevalence of asthma among children in the United States, 1976 to 1980. Pediatrics. 1988;81:1-7.

2. Centers for Disease Control. Surveillance for Asthma—United States, 1960-1995. Morbidity and Mortality Weekly Report. 1998;47:1-27.

3. Weiss K B, Gergen P J, Wagener D K. Breathing better or wheezing worse? The changing epidemiology of asthma morbidity and mortality. Annu Rev Public Health. 1993;14:491-513.

4. Sullivan S D, Strassels S A. Health Economics. In: Barnes P J, Drazen J M, Rennard S, Thomson N C, eds. Asthma and COPD. Basic Mechanisms and Clinical Management. San Diego: Academic Press; 2002:657-671.

5. Drazen J M, Silverman E K, Lee T H. Heterogeneity of therapeutic responses in asthma. Br Med Bull. 2000;56:1054-70.

6. Liggett S B. Pharmacogenetic applications of the Human Genome project. Nat Med. 2001;7:281-3.

7. Silverman E S, Hjoberg J, Palmer L J, Tantisira K G, Weiss S T, Drazen J M. Application of Pharmacogenetics to the Therapeutics of Asthma. In: Eissa NT, Huston D, eds. Therapeutic Targets of Airway Inflammation. 1000. New York: Marcel Dekker; 2002.

8. Ingelman-Sundberg M. Pharmacogenetics: an opportunity for a safer and more efficient pharmacotherapy. J Intern Med. 2001;250:186-200.

9. Lanes S F, Garcia Rodriguez L A, Huerta C. Respiratory medications and risk of asthma death. Thorax. 2002;57:683-6.

10. Dunlop K A, Carson D J, Shields M D. Hypoglycemia due to adrenal suppression secondary to high-dose nebulized corticosteroid. Pediatr Pulmonol. 2002;34:85-6.

11. Kennedy M J, Carpenter J M, Lozano R A, Castile R G. Impaired recovery of hypothalamic-pituitary-adrenal axis function and hypoglycemic seizures after high-dose inhaled corticosteroid therapy in a toddler. Ann Allergy Asthma Immunol. 2002;88:523-6.

12. Blumenthal M N. Historical Perspectives. In: Blumenthal M N, Bjorksten B, eds. Genetics of Allergy and Asthma: Methods for Investigative Studies. New York: Marcel Dekker; 1997:1-18.

13. Nieminen M M, Kaprio J, Koskenvuo M. A population-based study of bronchial asthma in adult twin pairs. Chest. 1991;100:70-5.

14. Duffy D L, Martin N G, Battistutta D, Hopper J L, Mathews J D. Genetics of asthma and hay fever in Australian twins. Am Rev Respir Dis. 1990;142:1351-8.

15. Ring H Z, Kroetz D L. Candidate gene approach for pharmacogenetic studies. Pharmacogenomics. 2002;3:47-56.

16. Malmstrom K, Rodriguez-Gomez G, Guerra J, Villaran C, Pineiro A, Wei L X, Seidenberg B C, Reiss T F. Oral montelukast, inhaled beclomethasone, and placebo for chronic asthma. A randomized, controlled trial. Montelukast/Beclomethasone Study Group. Ann Intern Med. 1999;130:487-95.

17. Israel E, Drazen J M, Liggett S B, Boushey H A, Chemiack R M, Chinchilli V M, Cooper D M, Fahy J V, Fish J E, Ford J G, Kraft M, Kunselman S, Lazarus S C, Lemanske R F, Martin R J, McLean D E, Peters S P, Silverman E K, Sorkness C A, Szefler S J, Weiss S T, Yandava C N. The effect of polymorphisms of the beta(2)-adrenergic receptor on the response to regular use of albuterol in asthma. Am J Respir Crit Care Med. 2000; 162:75-80.

18. Israel E, Drazen J M, Liggett S B, Boushey H A, Cherniack R M, Chinchilli V M, Cooper D M, Fahy J V, Fish J E, Ford J G, Kraft M, Kunselman S, Lazarus S C, Lemanske R F, Jr., Martin R J, McLean D E, Peters S P, Silverman E K, Sorkness C A, Szefler S J, Weiss S T, Yandava C N. Effect of polymorphism of the beta(2)-adrenergic receptor on response to regular use of albuterol in asthma. Int Arch Allergy Immunol. 2001; 124:183-6.

19. Liggett SB. Pharmacogenetics of beta-1- and beta-2-adrenergic receptors. Pharmacology. 2000;61:167-73.

20. Martinez FD, Graves PE, Baldini M, Solomon S, Erickson R. Association between genetic polymorphisms of the beta2-adrenoceptor and response to albuterol in children with and without a history of wheezing. J Clin Invest. 1997;100:3184-8.

21. Anderson W, Kalberg C, Edwards L, Fling M, Binnie C, Nguyen Q, Sherman P, Sprankle K, Wagner M, Rickard K, Manasco P. Effects of polymorphisms in the promoter region of 5-lipoxygenase and LTC4 synthase on the clinical response to zafirlukast and fluticasone. Eur Resir J. 2000;16(Suppl B):183s.

22. Drazen J M, Yandava C N, Dube L, Szczerback N, Hippensteel R, Pillari A, Israel E, Schork N, Silverman E S, Katz D A, Drajesk J. Pharmacogenetic association between ALOX5 promoter genotype and the response to anti-asthma treatment. Nat Genet. 1999;22: 168-70.

23. Palmer L J, Silverman E S, Weiss S T, Drazen J M. Pharmacogenetics of asthma. Am J Respir Crit Care Med. 2002;165:861-6.

24. Paltiel A D, Fuhlbrigge A L, Kitch B T, Liljas B, Weiss S T, Neumann P J, Kuntz K M. Cost-effectiveness of inhaled corticosteroids in adults with mild-to-moderate asthma: results from the asthma policy model. J Allergy Clin Immunol. 2001;108:39-49.

25. Aksoy M O, Mardini I A, Yang Y, Bin W, Zhou S, Kelsen S G. Glucocorticoid effects on the beta-adrenergic receptor-adenylyl cyclase system of human airway epithelium. J Allergy Clin Immunol. 2002;109:491-7.

26. Mak J C, Hisada T, Salmon M, Barnes P J, Chung K F. Glucocorticoids reverse IL-1beta-induced impairment of beta-adrenoceptor-mediated relaxation and up-regulation of G-protein-coupled receptor kinases. Br J. Pharmacol. 2002;135:987-96.

27. The Childhood Asthma Management Program (CAMP): design, rationale, and methods. Childhood Asthma Management Program Research Group. Controlled Clinical Trials. 1999;20:91-120.

28. Long-term effects of budesonide or nedocromil in children with asthma. The Childhood Asthma Management Program Research Group. N Engl J. Med. 2000;343: 1054-63.

29. Stephens M, Smith N J, Donnelly P. A new statistical method for haplotype reconstruction from population data. Am J Hum Genet. 2001;68:978-89.

30. Sham P C, Curtis D. Monte Carlo tests for associations between disease and alleles at highly polymorphic loci. Ann Hum Genet. 1995;59:97-105.

31. Schaid D J, Rowland C M, Tines D E, Jacobson R M, Poland G A. Score tests for association between traits and haplotypes when linkage phase is ambiguous. Am J Hum Genet. 2002;70:425-34.

32. Ramoni M, Sebastiani P. BEST: Best Enumeration of SNP Tags. In. 1.0 ed. Boston: Informatics Program, Children's Hospital, Harvard Medical School; 2002. http://genomethods.org/best/about.htm

33. Sun X, Ding H, Hung K, Guo B. A new MALDI-TOF based mini-sequencing assay for genotyping of SNPS. Nucleic Acids Res. 2000;28:E68.

34. Pritchard J K, Rosenberg N A. Use of unlinked genetic markers to detect population stratification in association studies. Am J Hum Genet. 1999;65:220-8.

35. Pritchard J K, Stephens M, Rosenberg N A, Donnelly P. Association mapping in structured populations. Am J Hum Genet. 2000;67:170-81.

36. Szefler, S. J. et al. Significant variability in response to inhaled corticosteroids for persistent asthma. J Allergy Clin Immunol 109, 410-8. (2002).

37. Johnson, G. C. et al. Haplotype tagging for the identification of common disease genes. Nat Genet 29, 233-7. (2001).

38. Palmer, L. J. & Cookson, W. O. Genomic approaches to understanding asthma. Genome Res 10, 1280-7. (2000).

39. Von Mutius, E. Presentation of new GINA guidelines for paediatrics. The Global Initiative on Asthma. Clin Exp Allergy 30 Suppl 1, 6-10. (2000).

40. Rossi, N. et al. Third International Pediatric Consensus statement on the management of childhood asthma. International Pediatric Asthma Consensus Group. [see comments.]. Allergy & Asthma Proceedings 22, 297-302 (2001).

41. NHLBI, Highlights of the Expert Panel Report 2. Guidelines for the Diagnosis and Management of Asthma, 97-4051A (NIH Publications, Bethesda, 1997).

42. Elenkov, I. J., Webster, E. L., Torpy, D. J. & Chrousos, G. P. Stress, corticotropin-releasing hormone, glucocorticoids, and the immune/inflammatory response: acute and chronic effects. Ann N YAcad Sci 876, 1-11; discussion 11-3. (1999).

43. Dautzenberg, F. M. & Hauger, R. L. The CRF peptide family and their receptors: yet more partners discovered. Trends Pharmacol Sci 23, 71-7. (2002).

44. Drolet, G. & Rivest, S. Corticotropin-releasing hormone and its receptors; an evaluation at the transcription level in vivo. Peptides 22, 761-7. (2001).

45. Habib, K. E. et al. Oral administration of a corticotropin-releasing hormone receptor antagonist significantly attenuates behavioral, neuroendocrine, and autonomic responses to stress in primates. Proc Natl Acad Sci USA 97, 6079-84. (2000).

46. Funada, M., Hara, C. & Wada, K. Involvement of corticotropin-releasing factor receptor subtype 1 in morphine withdrawal regulation of the brain noradrenergic system. Eur J Pharmacol 430, 277-81. (2001).

47. Theoharides, T. C. et al. Stress-induced intracranial mast cell degranulation: a corticotropin-releasing hormone-mediated effect. Endocrinology 136, 5745-50. (1995).

48. Sakai, K. et al. The genomic organization of the human corticotropin-releasing factor type-1 receptor. Gene 219, 125-30. (1998).

49. Pisarchik, A. & Slominski, A. T. Alternative splicing of CRH-R1 receptors in human and mouse skin: identification of new variants and their differential expression. Faseb J 15, 2754-6. (2001).

50. Liaw, C. W., Grigoriadis, D. E., Lovenberg, T. W., De Souza, E. B. & Maki, R. A. Localization of ligand-binding domains of human corticotropin-releasing factor receptor: a chimeric receptor approach. Mol Endocrinol 11, 980-5. (1997).

51. Dautzenberg, F. M., Kilpatrick, G. J., Hauger, R. L. & Moreau, J. Molecular biology of the CRH receptors—in the mood. Peptides 22, 753-60. (2001).

52. Xu, G. et al. Inhibition of corticotropin releasing hormone type-I receptor translation by an upstream AUG triplet in the 5′ untranslated region. Mol Pharmacol 59, 485-92. (2001).

53. Silverman, E. S. et al. Corticotropin-releasing hormone deficiency increases allergen-induced airway inflammation in a mouse model of asthma. Submitted (2003).

54. Lazarus, S. C. et al. Long-acting beta2-agonist monotherapy vs continued therapy with inhaled corticosteroids in patients with persistent asthma: a randomized controlled trial. Jama 285, 2583-93 (2001).

55. Lemanske, R. F., Jr. et al. Inhaled corticosteroid reduction and elimination in patients with persistent asthma receiving salmeterol: a randomized controlled trial. Jama 285, 2594-603 (2001).

Equivalents

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

We claim:

1. A method of assessing sensitivity to a therapeutic agent in a subject with a chronic obstructive pulmonary disease (COPD) or asthma, comprising:
determining a genotype of the subject, wherein the genotype of the subject is defined by a nucleotide sequence of a region of at least one gene selected from the group consisting of: ALOX15, CRH, CRHR1, CRHR2, urocortin, stresscopin, SRP (stresscopin-related peptide), CRHBP, EGR1, GATA3, HSD11B1, HSD11B2, MAPK8, NFATC4, SCYA11 (Eotaxin), FCER2 (CD23), IL18BP, ACTH (POMC), STAT3, STAT5A, STAT6, TBX21 (TBET) and TGF-β, and wherein the presence of a sequence variation in the region of the at least one gene is indicative of sensitivity to the therapeutic agent.

2. The method of claim 1, wherein the genotype of the subject is defined by a nucleotide sequence of a region of CRHR1.

3. The method of claim 1, wherein the sequence variation is a single nucleotide polymorphism.

4. The method of claim 3, wherein the single nucleotide polymorphism is in a nucleotide sequence found in a region of CRHR1, wherein the nucleotide sequence comprises a polymorphism of rs1876828, rs242939 or rs242941.

5. The method of claim 1, wherein the sequence variation is selected from the group consisting of a deletion and insertion.

6. The method of claim 1, wherein the sequence variation indicates a haplotype.

7. The method of claim 6, wherein the haplotype is defined by a nucleotide sequence which comprises a polymorphism of rs1876828, rs242939 or rs242941.

8. The method of claim 7, wherein the haplotype is defined by a nucleotide sequence which comprises a polymorphism of rs1876828 or rs242939.

9. The method of claim 7, wherein the haplotype is defined by a nucleotide sequence which comprises a polymorphism of rs242939 and rs242941.

10. The method of claim 7, wherein the haplotype is defined by a nucleotide sequence which comprises a polymorphism of rs1876828 and rs242941.

11. The method of claim 7, wherein the haplotype is defined by a nucleotide sequence which comprises a polymorphism of rs1876828, rs242939 and rs242941.

12. The method of claim 11, wherein the haplotype is defined by a nucleotide sequence which comprises a polymorphism of rs1876828, rs242939 and rs242941, and wherein the haplotype is the GAT haplotype.

13. The method of claim 12, wherein the subject is homozygous for the GAT haplotype.

14. The method of claim 1, wherein the sequence variation is determined with a method selected from the group consisting of nucleic acid hybridization and nucleic acid amplification.

15. The method of claim 14, wherein the nucleic acid hybridization is performed using a nucleic acid probe.

16. The method of claim 14, wherein the nucleic acid hybridization is performed using a nucleic acid microarray.

17. The method of claim 14, wherein the nucleic acid amplification is performed using PCR.

18. The method of claim 1, wherein the therapeutic agent is an inhaled corticosteroid.

19. The method of claim 1, wherein the therapeutic agent is a bronchodilator (beta-agonist).

20. The method of claim 1, wherein the COPD is selected from the group consisting of chronic bronchitis, emphysema, bronchioectasis and extrinsic allergic alveolitis.

21. The method of claim 1, wherein the subject has asthma.

22. The method of claim 1, wherein the presence of the sequence variation is indicative that the subject is not sensitive to the therapeutic agent.

23. The method of claim 1, further comprising assessing at least one risk factor to assess the sensitivity to the therapeutic agent.

24. The method of claim 12, wherein the risk factor is selected from the group consisting of baseline level of lung function, gender, age, race and prior steroid use.

25. A method of assessing sensitivity to a corticosteroid or beta-agonist in a subject, comprising:
determining a genotype of the subject, wherein the genotype of the subject is defined by a nucleotide sequence of a region of at least one gene selected from the group consisting of: ALOX15, CRH, CRHR1, CRHR2, urocortin, stresscopin, SRP (stresscopin-related peptide), CRHBP, EGR1, GATA3, HSD11B1, HSD11B2, MAPK8, NFATC4, SCYA1 (Eotaxin), FCER2 (CD23), IL18BP, ACTH (POMC), STAT3, STAT5A, STAT6, TBX21 (TBET) and TGF-β, and wherein the presence of a sequence variation in the region of the at least one gene is indicative of sensitivity to the corticosteroid or beta-agonist.

26. The method of claim 25, wherein the subject has COPD or asthma.

27. The method of claim 25, wherein the subject suffers from depression.

28. The method of claim 25, wherein the genotype of the subject is defined by a nucleotide sequence of a region of CRHR1.

29. The method of claim 25, wherein the sequence variation is a single nucleotide polymorphism.

30. The method of claim 29, wherein the single nucleotide polymorphism is in a nucleotide sequence found in a region of CRHR1, wherein the nucleotide sequence comprises a polymorphism of rs1876828, rs242939 or rs242941.

31. The method of claim 25, wherein the sequence variation is selected from the group consisting of a deletion and insertion.

32. The method of claim 25, wherein the sequence variation indicates a haplotype.

33. The method of claim 32, wherein the haplotype is defined by a nucleotide sequence which comprises a polymorphism of rs1876828, rs242939 or rs242941.

34. The method of claim 25, wherein the sequence variation is determined with a method selected from the group consisting of nucleic acid hybridization and nucleic acid amplification.

35. The method of claim 34, wherein the nucleic acid hybridization is performed using a nucleic acid probe.

36. The method of claim 34, wherein the nucleic acid hybridization is performed using a nucleic acid microarray.

37. The method of claim 34, wherein the nucleic acid amplification is performed using PCR.

38. A method of assessing sensitivity to a therapeutic agent in a subject with a chronic obstructive pulmonary disease (COPD) or asthma, comprising:
determining a genotype of the subject, wherein the genotype of the subject is defined by a nucleotide sequence of a region of NR3C1, and wherein the presence of a sequence variation in the region of NR3C1 is indicative of sensitivity to the therapeutic agent.

39. The method of claim 38, wherein the nucleotide sequence is selected from the group consisting of nucleotide sequences set forth as SEQ ID NOs: 89-121 and 309-324.

39. The method of claim 38, wherein the nucleotide sequence is selected from the group consisting of nucleotide sequences set forth as SEQ ID NOs: 89 and 100.

40. The method of claim 38, wherein the sequence variation is a single nucleotide polymorphism.

41. The method of claim 38, wherein the sequence variation is selected from the group consisting of a deletion and insertion.

41. The method of claim 38, wherein the sequence variation indicates a haplotype.

43. The method of claim 38, wherein the sequence variation is determined with a method selected from the group consisting of nucleic acid hybridization and nucleic acid amplification.

44. The method of claim 43, wherein the nucleic acid hybridization is performed using a nucleic acid probe.

45. The method of claim 43, wherein the nucleic acid hybridization is performed using a nucleic acid microarray.

46. The method of claim 43, wherein the nucleic acid amplification is performed using PCR.

47. The method of claim 38, wherein the therapeutic agent is an inhaled corticosteroid.

48. The method of claim 38, wherein the therapeutic agent is a bronchodilator.

49. The method of claim 38, wherein the COPD is selected from the group consisting of chronic bronchitis, emphysema, bronchioectasis and extrinsic allergic alveolitis.

50. The method of claim 38, wherein the subject has asthma.

51. The method of claim 38, wherein the presence of the sequence variation is indicative that the subject is not sensitive to the therapeutic agent.

52. The method of claim 38, further comprising assessing at least one risk factor to assess the sensitivity to the therapeutic agent.

53. The method of claim 52, wherein the risk factor is selected from the group consisting of baseline level of lung function, gender, age, race and prior steroid use.

54. A method of assessing sensitivity to a therapeutic agent in a subject with chronic obstructive pulmonary disease (COPD) or asthma, comprising:
detecting the presence of a nucleic acid molecule in a biological sample from the subject, wherein the nucleic acid molecule is selected from the group consisting of nucleotide sequences of a region of a gene selected from the group consisting of: ALOX15, CRH, CRHR1, CRHR2, urocortin, stresscopin, SRP (stresscopin-related peptide), CRHBP, EGR1, GATA3, HSD1 IBI, HSD11B2, MAPK8, NFATC4, SCYA11 (Eotaxin), FCER2 (CD23), IL18BP, ACTH (POMC), STAT3, STAT5A, STAT6, TBX21 (TBET) and TGF-, which contains a sequence variation,
(a) nucleotide sequences set forth as SEQ ID NOs: 1-88, 122-308 and 325-374, and
(b) fragments of (a), wherein the fragment of the nucleotide sequences contains a sequence variation,
and wherein the presence of a sequence variation in the nucleic acid molecule is indicative of sensitivity to the therapeutic agent.

55. The method of claim 54, wherein the nucleic acid molecule is selected from the group consisting of nucleotide sequences set forth as SEQ ID NOs: 1-35 and fragments thereof, which contains a sequence variation.

56. The method of claim 54, wherein the nucleic acid molecule is selected from the group consisting of nucleotide sequences of a region of CRHR1, and wherein the nucleotide sequence is selected from the group consisting of SEQ ID NOs: 205-208.

57. The method of claim 54, wherein the nucleic acid molecule is selected from the group consisting of nucleotide sequences of a region of IL18BP, wherein the nucleotide sequence is selected from the group consisting of SEQ ID NOs: 209-212.

58. The method of claim 54, wherein the nucleic acid molecule is selected from the group consisting of nucleotide sequences of a region of FCER2, wherein the nucleotide sequence is selected from the group consisting of SEQ ID NOs: 213-222.

59. The method of claim 54, wherein the nucleic acid molecule is selected from the group consisting of nucleotide sequences of a region of CRHR1, wherein the sequence comprises the polymorphisms of rs1876828, rs242939 and rs242941.

60. The method of claim 54, wherein the nucleic acid molecule is selected from the group consisting of nucleotide sequences of a region of IL18BP, wherein the sequence comprises the polymorphisms of rs1892919, G9772a3 and G9772a6.

61. The method of claim 54, wherein the nucleic acid molecule is selected from the group consisting of nucleotide sequences of a region of FCER2, wherein the sequence comprises the polymorphisms of G9782a12, G9782a19, G9782a26, G9782a5 and G9782a8.

62. The method of claim 54, wherein the presence of the nucleic acid molecule is determined with a method selected from the group consisting of nucleic acid hybridization and nucleic acid amplification.

63. The method of claim 62, wherein the nucleic acid hybridization is performed using a nucleic acid probe.

64. The method of claim 62, wherein the nucleic acid hybridization is performed using a nucleic acid microarray.

65. The method of claim 62, wherein the nucleic acid amplification is performed using PCR.

66. The method of claim 54, wherein the biological sample is a blood sample.

67. The method of claim 54, wherein the nucleic acid molecule is genomic DNA.

68. The method of claim 54, wherein the nucleic acid molecule is mRNA.

69. The method of claim 54, wherein the therapeutic agent is an inhaled corticosteroid.

70. The method of claim 54, wherein the therapeutic agent is a bronchodilator.

71. The method of claim 54, wherein the COPD is selected from the group consisting of chronic bronchitis, emphysema, bronchioectasis and extrinsic allergic alveolitis.

72. The method of claim 54, wherein the subject has asthma.

73. The method of claim 54, wherein the presence of the sequence variation is indicative that the subject is not sensitive to the therapeutic agent.

74. The method of claim 54, further comprising assessing at least one risk factor to assess the sensitivity to the therapeutic agent.

75. The method of claim 74, wherein the risk factor is selected from the group consisting of baseline level of lung function, gender, age, race and prior steroid use.

76. A method of assessing sensitivity to a therapeutic agent in a subject with a chronic obstructive pulmonary disease (COPD) or asthma, comprising:
determining the presence of a mutant protein encoded by a nucleic acid molecule selected from the group consisting of: nucleotide sequences of a region of a gene selected from the group consisting of: ALOX15, CRH, CRHR1, CRHR2, urocortin, stresscopin, SRP (stresscopin-related peptide), CRHBP, EGR1, GATA3, HSD11B1, HSD11B2, MAPK8, NFATC4, SCYA11 (Eotaxin), FCER2 (CD23), IL18BP, ACTH (POMC), STAT3, STAT5A, STAT6, TBX21 (TBET) and TGF-β, which contains a sequence variation,
(a) nucleotide sequences set forth as SEQ ID NOs: 1-88, 122-308 and 325-374, and
(b) fragments of (b), which contain a sequence variation,
wherein the presence of the mutant protein is indicative of sensitivity to the therapeutic agent.

77. The method of claim 76, wherein the nucleotide sequence is of a region of CRHR1, which contains a sequence variation.

78. The method of claim 76, wherein the nucleotide sequence is a nucleotide sequence which comprises a polymorphism of rs1876828, rs242939 or rs242941.

79. The method of claim 76, wherein the nucleic acid molecule is selected from the group consisting of nucleotide sequences set forth as SEQ ID NOs: 1-35 and fragments thereof, which contain a sequence variation.

80. The method of claim 76, wherein the presence of the mutant protein is detected with an agent that selectively binds to the mutant protein.

81. The method of claim 80, wherein the agent that selectively binds is a binding polypeptide.

82. The method of claim 81, wherein the binding polypeptide is an antibody or an antigen-binding fragment thereof.

83. The method of claim 82, wherein the antibody is bound to a detectable label.

84. The method of claim 83, wherein the detectable label is a fluorescent molecule.

85. The method of claim 76, wherein the therapeutic agent is an inhaled corticosteroid.

77. A kit, comprising:
one or more nucleic acid probes that hybridize to at least one nucleic acid molecule selected from the group consisting of:
(a) nucleotide sequences of a region of a gene selected from the group consisting of: ALOX15, CRH, CRHR1, CRHR2, urocortin, stresscopin, SRP (stresscopin-related peptide), CRHBP, EGR1, GATA3, HSD11B1, HSD11B2, MAPK8, NFATC4, SCYA11 (Eotaxin), FCER2 (CD23), IL18BP, ACTH (POMC), STAT3, STAT5A, STAT6, TBX21 (TBET) and TGF-β, which contain a sequence variation,
(b) nucleotide sequences set forth as SEQ ID NOs: 1-88, 122-308 and 325-374, and
(c) fragments of (a), wherein the fragment of the nucleotide sequence contains a sequence variation, and
instructions for the use of the nucleic acid probes to correlate the presence of the at least one nucleic acid molecule with sensitivity to a therapeutic agent.

78. The kit of claim 77, wherein the nucleotide sequence is of a region of CRHR1, which contains a sequence variation.

79. The kit of claim 77, wherein the nucleotide sequence is a nucleotide sequence which comprises a polymorphism of rs1876828, rs242939 or rs242941.

80. The kit of claim 77, further comprising one or more control agents.

81. The kit of claim 77, wherein the one or more nucleic acidw consist of a first primer and a second primer, wherein the first primer and the second primer are constructed and arranged to selectively amplify a region of the nucleic acid molecule which contains a sequence variation.

82. The kit of claim 77 or 81, wherein the nucleic acid molecule is selected from the group consisting of nucleotide sequences set forth as SEQ ID NOs: 1-35.

83. The kit of claim 80, wherein the one or more nucleic acid probes and one or more control agents are bound to a substrate.

84. A kit, comprising:
one or more nucleic acid probes that hybridize to at least one nucleic acid molecule selected from the group consisting of:
(d) nucleotide sequences of a region of a NR3C1, which contain a sequence variation,
(e) nucleotide sequences set forth as SEQ ID NOs: 89-121 and 309-324, and
(f) fragments of (a), wherein the fragment of the nucleotide sequence contains a sequence variation, and
instructions for the use of the nucleic acid probes to correlate the presence of the at least one nucleic acid molecule with sensitivity to a therapeutic agent.

85. The kit of claim 84, further comprising one or more control agents.

86. The kit of claim 84, wherein the at least one nucleic acid molecule is selected from the group consisting of nucleotide sequences set forth as SEQ ID NOs: 89 and 100.

87. The kit of claim 84, wherein the one or more nucleic acid consist of a first primer and a second primer, wherein the first primer and the second primer are constructed and arranged to selectively amplify a region of the nucleic acid molecule which contains a sequence variation.

88. The kit of claim 84 or 87, wherein the nucleic acid molecule is selected from the group consisting of nucleotide sequences set forth as SEQ ID NOs: 89 and 100.

89. The kit of claim 85, wherein the one or more nucleic acid probes and one or more control agents are bound to a substrate.

90. A kit, comprising:
one or more nucleic acid probes that hybridize to at least one nucleic acid molecule selected from the group consisting of:
(g) nucleotide sequences of a region of a NR3C₁, which contain a sequence variation,
(h) nucleotide sequences set forth as SEQ ID NOs: 89-121 and 309-324, and
(i) fragments of (a), wherein the fragment of the nucleotide sequence contains a sequence variation, and
instructions for the use of the nucleic acid probes to correlate the presence of the at least one nucleic acid molecule with sensitivity to a therapeutic agent.

91. The kit of claim 90, further comprising one or more control agents.

92. The kit of claim 90, wherein the at least one nucleic acid molecule is selected from the group consisting of nucleotide sequences set forth as SEQ ID NOs: 89 and 100.

93. The kit of claim 90, wherein the one or more nucleic acid consist of a first primer and a second primer, wherein the first primer and the second primer are constructed and arranged to selectively amplify a region of the nucleic acid molecule which contains a sequence variation.

94. The kit of claim 90 or 93, wherein the nucleic acid molecule is selected from the group consisting of nucleotide sequences set forth as SEQ ID NOs: 89 and 100.

95. The kit of claim 91, wherein the one or more nucleic acid probes and one or more control agents are bound to a substrate.

96. A kit, comprising:
one or more binding polypeptides that selectively bind to a mutant protein encoded by a nucleic acid molecule selected from the group consisting of:
(a) nucleotide sequences of a gene selected from the group consisting of: ALOX15, CRH, CRHR1, CRHR2, urocortin, stresscopin, SRP (stresscopin-related peptide), CRHBP, EGR1, GATA3, HSD11B1, HSD11B2, MAPK8, NFATC4, SCYA11 (Eotaxin), FCER2 (CD23), IL18BP, ACTH (POMC), STAT3, STAT5A, STAT6, TBX21 (TBET) and TGF-β, which contain a sequence variation,
(b) nucleotide sequences set forth as SEQ ID NOs: 1-88, 122-308 and 325-374, and
(c) fragments of (a) and (b), wherein the fragment of the nucleotide sequence contains a sequence variation, and
instructions for the use of the one or more binding polypeptides to correlate the presence of the mutant protein with sensitivity to a therapeutic agent.

97. The kit of claim 96, wherein the nucleotide sequence is of a region of CRHR1, which contains a sequence variation.

98. The kit of claim 96, wherein the nucleotide sequence is a nucleotide sequence which comprises a polymorphism of rs1876828, rs242939 or rs242941.

99. The kit of claim 96, further comprising one or more control agents.

100. The kit of claim 96, wherein the nucleic acid molecule is selected from the group consisting of nucleotide sequences set forth as SEQ ID NOs: 1-35 and fragments thereof, which contain a sequence variation.

101. The kit of claim 96, wherein the one or more binding polypeptides are antibodies or antigen-binding fragments thereof.

102. The kit of claim 101, wherein the antibodies or antigen-binding fragments thereof are bound to a substrate.

103. A nucleic acid microarray comprising at least two different nucleic acid molecules that hybridize to a nucleotide sequence selected from the group consisting of
(a) nucleotide sequences of a region of a gene selected from the group consisting of: ALOX15, CRH, CRHR1, CRHR2, urocortin, stresscopin, SRP (stresscopin-related peptide), CRHBP, EGR1, GATA3, HSD11B1, HSD11B2, MAPK8, NFATC4, SCYA11 (Eotaxin), FCER2 (CD23), IL18BP, ACTH (POMC), STAT3, STAT5A, STAT6, TBX21 (TBET) and TGF-β, which contain a sequence variation,
(b) nucleotide sequences set forth as SEQ ID NOs: 1-88, 122-308 and 325-374 and
(c) fragments of (a), wherein the fragment of the nucleotide sequence contains a sequence variation,
and wherein the at least two nucleic acid molecules are fixed to a solid subtrate.

104. The nucleic acid microarray of claim 103, wherein the nucleotide sequence is of a region of CRHR1.

105. The nucleic acid microarray of claim 103, wherein the nucleotide sequence is a nucleotide sequence which comprises a polymorphism of rs1876828, rs242939 or rs242941.

106. The nucleic acid microarray of claim 103, wherein the nucleotide sequence is selected from the group consisting of nucleotide sequences set forth as: SEQ ID NOs: 1-35 and fragments thereof, which contain a sequence variation.

107. The nucleic acid microarray of claim 103, wherein at least ten different nucleic acid molecules are fixed to the solid substrate.

108. The nucleic acid microarray of any one of claims 103-107, further comprising at least one control nucleic acid molecule.
